(19) United States
(12) Patent Application Publication
(10) Pub. No.: US 2026/0108276 A1
(43) Pub. Date: Apr. 23, 2026

(54) SYSTEMS AND METHODS FOR TRANSPORT AND DISTRACTION

(71) Applicant: BIODYNAMIK, INC., Lake Forest, CA (US)

(72) Inventors: EMMON JOHNNY CHEN, Aliso Viejo, CA (US); SHANBAO CHENG, Rancho Santa Margarita, CA (US)

(21) Appl. No.: 19/162,038

(22) PCT Filed: Dec. 4, 2024
(86) PCT No.: PCT/US24/58493

§ 371 Date: Sep. 3, 2025

Related U.S. Application Data
(63) Continuation-in-part of application No. 18/932,376, filed on Oct. 30, 2024, now Pat. No. 12,453,582.
(63) Continuation-in-part of application No. 18/932,397, filed on Oct. 30, 2024, now Pat. No. 12,453,583.
Application No. PCT/US24/58493 is a continuation-in-part of application No. 18/932,397, filed on Oct. 30, 2024, now Pat. No. 12,453,583.
(60) Provisional application No. 63/606,038, filed on Dec. 4, 2023.
(60) Provisional application No. 63/606,038, filed on Dec. 4, 2023.
(60) Provisional application No. 63/554,970, filed on Feb. 17, 2024.
(60) Provisional application No. 63/554,970, filed on Feb. 17, 2024.
(60) Provisional application No. 63/656,900, filed on Jun. 6, 2024.
(60) Provisional application No. 63/656,900, filed on Jun. 6, 2024.
(60) Provisional application No. 63/677,610, filed on Jul. 31, 2024.
(60) Provisional application No. 63/677,610, filed on Jul. 31, 2024.
(60) Provisional application No. 63/677,610, filed on Jul. 31, 2024.
(60) Provisional application No. 63/677,610, filed on Jul. 31, 2024.

(51) Int. Cl. A61B 17/66; A61B 17/00; A61B 17/56

(52) U.S. Cl. CPC A61B 17/66; A61B 2017/00084; A61B 2017/00477; A61B 2017/564

(57) ABSTRACT

A transport device for increasing biological activity within a patient includes a base including a first end, a second end, an upper surface located between the first end and the second end, and a lower surface located between the first end and the second end, a base anchor configured to statically couple the base to a first portion of a bone of a subject, a translatable anchor configured to engage a growth stimulator, a leadscrew dynamically coupling the base to the translatable anchor, wherein the leadscrew is configured to rotate about a leadscrew axis while substantially maintaining its longitudinal position along the leadscrew axis in relation to the base, such that the growth stimulator, when engaged with the translatable anchor, is capable of independent movement in relation to the base along a stimulation axis that includes at least some transverse displacement with respect to the bone when the base is coupled to the bone via the base anchor, and a screw drive rotatably coupled to the leadscrew and configured to couple to a rotatable mating tool that is configured to rotate the leadscrew via the screw drive.

### INCORPORATION BY REFERENCE TO ANY PRIORITY APPLICATIONS

The following U.S. patent applications are hereby incorporated by reference in their entirety for all purposes: U.S. Provisional Patent Application No. 63/554,970, filed on Feb. 17, 2024, U.S. Provisional Patent Application No. 63/656,900, filed on Jun. 6, 2024, U.S. Provisional Patent Application No. 63/677,610, filed on Jul. 31, 2024, U.S. patent application Ser. No. 18/932,376, filed on Oct. 30, 2024, and U.S. patent application Ser. No. 18/932,397, filed on Oct. 30, 2024.

### BACKGROUND OF THE INVENTION

### Field of the Invention

This invention relates to devices and methods for distracting a bone cortex or bone segment, or soft tissues such as periosteum, for transverse transport or tissue stretching, and more specifically to devices and methods for internal or external application of transverse transport (TT) for increasing blood flow and inducing tissue and/or bone regeneration. Transverse transport (TT) includes tibial cortex transverse transport (TTT), or transverse transport of other bone pieces, and/or periosteal distraction (PD) or other stretching.

### SUMMARY OF THE INVENTION

In one embodiment of the present disclosure, a system for increasing biological activity within a patient includes a housing, a drive carried within the housing, an indexing contour carried by the housing and configured to substantially stabilize the housing in relation to a transport device base of a transport device configured to move a growth stimulator in relation to a bone of a subject, a locking portion configured to releasably lock the housing to the transport device base, a rotatable mating tool carried by the housing, wherein the drive is configured to cause the rotatable mating tool to rotate, and wherein the rotatable mating tool is configured to be rotationally coupled to a screw drive of a leadscrew of the transport device when the housing is stabilized with the transport device base via the indexing contour and is locked to the transport device base via the locking portion, and a control unit configured to output a control signal configured to direct the drive to rotate the rotatable mating tool.

In another embodiment of the present disclosure, a method for increasing biological activity within a patient includes, providing a system including a housing, a drive carried within the housing, an indexing contour carried by the housing and configured to substantially stabilize the housing in relation to a transport device base of a transport device configured to move a growth stimulator in relation to a bone of a subject, a locking portion configured to releasably lock the housing to the transport device base, a rotatable mating tool carried by the housing, wherein the drive is configured to cause the rotatable mating tool to rotate, and wherein the rotatable mating tool is configured to be rotationally coupled to a screw drive of a leadscrew of the transport device when the housing is stabilized with the transport device base via the indexing contour and is locked to the transport device base via the locking portion, and a control unit configured to output a control signal configured to direct the drive to rotate the rotatable mating tool, surgically securing the transport device base to the bone of the subject, surgically placing the growth stimulator within the patient and coupling the growth stimulator to the transport device base such that it is displaceable relative to the transport device base via rotation of the leadscrew, engaging the indexing contour of the housing with the transport device base, locking the housing to the transport device base via the locking portion, coupling the rotatable mating tool to the screw drive of the leadscrew, and initiating the control signal to cause the rotatable mating tool rotate the leadscrew and to displace the growth stimulator in relation to the bone of the subject.

In still another embodiment of the present disclosure, a transport device for increasing biological activity within a patient includes a base including a first end, a second end, an upper surface located between the first end and the second end, and a lower surface located between the first end and the second end, a base anchor configured to statically couple the base to a first portion of a bone of a subject, a translatable anchor configured to engage a growth stimulator, a leadscrew dynamically coupling the base to the translatable anchor, wherein the leadscrew is configured to rotate about a leadscrew axis while substantially maintaining its longitudinal position along the leadscrew axis in relation to the base, such that the growth stimulator, when engaged with the translatable anchor, is capable of independent movement in relation to the base along a stimulation axis that includes at least some transverse displacement with respect to the bone when the base is coupled to the bone via the base anchor, and a screw drive rotatably coupled to the leadscrew and configured to couple to a rotatable mating tool that is configured to rotate the leadscrew via the screw drive.

In yet another embodiment of the present disclosure, a method for increasing biological activity within a patient includes providing a transport device including a base including a first end, a second end, an upper surface located between the first end and the second end, and a lower surface located between the first end and the second end, a base anchor configured to statically couple the base to a first portion of a bone of a subject, a translatable anchor configured to engage a growth stimulator, a leadscrew dynamically coupling the base to the translatable anchor, wherein the leadscrew is configured to rotate about a leadscrew axis while substantially maintaining its longitudinal position along the leadscrew axis in relation to the base, such that the growth stimulator, when engaged with the translatable anchor, is capable of independent movement in relation to the base along a stimulation axis that includes at least some transverse displacement with respect to the bone when the base is coupled to the bone via the base anchor, and a screw drive rotatably coupled to the leadscrew and configured to couple to a rotatable mating tool that is configured to rotate the leadscrew via the screw drive, surgically securing the base anchor to the bone of the subject, surgically coupling the translatable anchor to the growth stimulator within the subject, coupling the rotatable mating tool to the screw drive of the leadscrew, and causing the leadscrew to rotate via the rotatable mating tool to displace the translatable anchor in relation to the base and to cause the growth stimulator to move with at least a transverse directional component in relation to the bone of the subject.

In still another embodiment of the present disclosure, a transport device for increasing biological activity within a patient includes a base including a first end and a second end, a base anchor configured to statically couple the base to a first portion of a bone of a subject, a stage having a first end and a second end and configured to engage a growth stimulator, a first angled surface carried on the first end of the base, a second angled surface carried on the first end of the stage, a first wedge having a third angled surface configured for sliding contact with the first angled surface and a fourth angled surface configured for sliding contact with the second angled surface, and a drive configured to move the first wedge in relation to the base and the stage, wherein movement of the first wedge in a first direction causes the base and the stage to move apart from each other via simultaneous sliding of the third angled surface with the first angled surface and sliding of the fourth angled surface with the second angled surface.

In yet another embodiment of the present disclosure, a transport device for increasing biological activity within a patient includes a base including a first end and a second end, a base anchor configured to statically couple the base to a first portion of a bone of a subject, a stage having a first end and a second end and configured to engage a growth stimulator, the stage including a first laterally-extending projection, a shuttle configured for longitudinal movement on or in the base, the shuttle including a vertically-extending wall having a slanted groove, the first laterally-extending projection configured to slidingly engage the groove, and a drive configured to move the shuttle in a first longitudinal direction, wherein movement of the shuttle in the first longitudinal direction causes the first laterally-extending projection to be moved within the groove the movement including at least some upward movement, and wherein the stage is caused to be moved along with the first laterally-extending projection.

In still another embodiment of the present disclosure, a transport device for increasing biological activity within a patient includes a base including a first end, a second end, an upper surface located between the first end and the second end, and a lower surface located between the first end and the second end, a base anchor configured to statically couple the base to a first portion of a bone of a subject, a stage configured to engage a growth stimulator, a drive coupled to the base and configured to output rotation to a pinion, and a rack coupled to the stage, wherein the pinion is configured to engage the rack and to lift the stage as the pinion is turned in a first direction.

In yet another embodiment of the present disclosure, a transport device for increasing biological activity within a patient includes a base including a first end, a second end, an upper surface located between the first end and the second end, and a lower surface located between the first end and the second end, a base anchor configured to statically couple the base to a first portion of a bone of a subject, a stage configured to engage a growth stimulator, a drive coupled to the stage and configured to output rotation to a pinion; and a rack coupled to the base, wherein the pinion is configured to engage the rack and to lift the stage as the pinion is turned in a first direction.

### BRIEF DESCRIPTION OF THE DRAWINGS

FIG. 1 is a perspective view of the anatomy of a tibia of a patient.

FIG. 2 is a side view of a lower leg of a patient suffering from a diabetic foot ulcer.

FIG. 3 illustrates a transverse tibia transport device in a first position in use, according to an embodiment of the present disclosure.

FIG. 4 illustrates a transverse tibia transport device in a second position in use, according to an embodiment of the present disclosure.

FIG. 5 illustrates a periosteal distraction device in a first position in use, according to an embodiment of the present disclosure.

FIG. 6 illustrates a periosteal distraction device in a second position in use, according to an embodiment of the present disclosure.

FIG. 7 is a perspective view of a transverse transport device, according to an embodiment of the present disclosure.

FIG. 8 is a perspective view of the transverse transport device of FIG. 7.

FIG. 9 is an exploded view of the transverse transport device of FIG. 7.

FIG. 10 is a top view of the base of the transverse transport device of FIG. 7.

FIG. 11 is a bottom view of the base of the transverse transport device of FIG. 7.

FIG. 12 is a cross-sectional view of the base taken along line 12 of FIG. 11.

FIG. 13 is a detailed cross-sectional view similar to that of FIG. 12, but with the components of the transverse transport device included.

FIG. 14 is an exploded view of the transverse transport device of FIG. 7.

FIG. 15 is an internal view of the leadscrew of the transverse transport device at a first rotational orientation, according to an embodiment of the present disclosure.

FIG. 16 is an internal view of the leadscrew of the transverse transport device at a second rotational orientation, according to an embodiment of the present disclosure.

FIG. 17 is a first perspective view of a system for increasing biological activity within a patient, according to an embodiment of the present disclosure.

FIG. 18 is a second perspective view of the system of FIG. 17.

FIG. 19 is a perspective view of a coupling step of a transverse transport device and a modular automated drive unit, according to an embodiment of the present disclosure.

FIG. 20 is an exploded view of a drive unit of the system of FIG. 17, according to an embodiment of the present disclosure.

FIG. 21 illustrates the system of FIG. 17 in a first position in use, according to an embodiment of the present disclosure.

FIG. 22 illustrates the system of FIG. 17 in a second position in use, according to an embodiment of the present disclosure.

FIG. 23 illustrates a transverse transport device in use, coupled to an external fixator frame, according to an embodiment of the present disclosure.

FIG. 24 illustrates a system for increasing biological activity within a patient in use, coupled to an external fixator frame, according to an embodiment of the present disclosure.

FIG. 25 is a perspective view of a periosteal distraction device, according to an embodiment of the present disclosure.

FIG. 26 is an exploded view of the periosteal distraction device of FIG. 25.

FIG. 27 is a first perspective view of a periosteal distraction device connected to an insertion tool, according to an embodiment of the present disclosure.

FIG. 28 is a second perspective view of a periosteal distraction device connected to an insertion tool, according to an embodiment of the present disclosure.

FIG. 29 is an exploded view of the insertion tool, according to an embodiment of the present disclosure.

FIG. 30 is a perspective view of a movable stage loaded onto a first portion of the insertion tool, according to an embodiment of the present disclosure.

FIG. 31 is a front view of the periosteal distraction device of FIG. 25 showing a first distraction amount, according to an embodiment of the present disclosure.

FIG. 32 is a front view of the periosteal distraction device of FIG. 25 showing a second distraction amount, according to an embodiment of the present disclosure.

FIG. 33 is a plan view of a first step for preparing a patient for implantation of a periosteal distraction device, according to an embodiment of the present disclosure.

FIG. 34 is a plan view of a second step for preparing a patient for implantation of a periosteal distraction device, according to an embodiment of the present disclosure.

FIG. 35 is a plan view of a third step for preparing a patient for implantation of a periosteal distraction device, according to an embodiment of the present disclosure.

FIG. 36 is a plan view of a fourth step for preparing a patient for implantation of a periosteal distraction device, according to an embodiment of the present disclosure.

FIG. 37 is a perspective view of a movable stage being inserted utilizing an insertion tool, according to an embodiment of the present disclosure.

FIG. 38 is a perspective view of bone anchors being inserted through targeting holes in an insertion tool, according to an embodiment of the present disclosure.

FIG. 39 is a perspective view of other preparation operations being performed through an insertion tool, according to an embodiment of the present disclosure.

FIG. 40 is a perspective view of the disassembly and removal of an insertion tool, according to an embodiment of the present disclosure.

FIG. 41 is a perspective view of a periosteal distraction device body and leadscrew being prepared for coupling with bone anchors and a movable stage, according to an embodiment of the present disclosure.

FIG. 42 is a perspective view of a body of a periosteal distraction device being secured to bone anchors, according to an embodiment of the present disclosure.

FIG. 43 is a perspective view of an alternative periosteal distraction device incorporating the base of FIGS. 7-8, being implanted within a patient, according to an embodiment of the present disclosure.

FIG. 44 is a perspective view of an alternative periosteal distraction device implanted within a patient, according to an embodiment of the present disclosure.

FIG. 45 is an exploded view of a targeting tool used with the periosteal distraction device of FIG. 44 in an insertion procedure, according to an embodiment of the present disclosure.

FIG. 46 is a schematic view of a communication hierarchy of a system for increasing biological activity within a patient, according to an embodiment of the present disclosure.

FIG. 47 is a partial sectional view of basic components, according to an embodiment of the present disclosure.

FIG. 48 is an exploded perspective view of a device that employs an angular opening, according to an embodiment of the present disclosure.

FIG. 49 is a perspective view of a device comprising bar linkage mechanisms for achieving motion of the intercalary plane away from the starting plane for distraction, according to an embodiment of the present disclosure.

FIG. 50 is a perspective view of a device comprising bar linkage mechanisms, according to an embodiment of the present disclosure.

FIG. 51 is a perspective view of a device comprising bar linage mechanisms, according to an embodiment of the present disclosure.

FIG. 52 is a side view of a device comprising bar linkage mechanisms, in a first position, according to an embodiment of the present disclosure.

FIG. 53 is a side view of a device comprising bar linkage mechanisms, in a second position, according to an embodiment of the present disclosure.

FIG. 54 is a perspective view of a device having an exemplary nut and lead screw mechanism, according to an embodiment of the present disclosure.

FIG. 55 is a perspective view of a device having an exemplary nut and lead screw mechanism, according to an embodiment of the present disclosure.

FIG. 56 is a perspective view of a device having an exemplary nut and lead screw mechanism, according to an embodiment of the present disclosure.

FIG. 57 is a perspective detail view of a device having a locking mechanism to prevent unintended movement, according to an embodiment of the present disclosure.

FIG. 58 is an elevation detail view of the device of FIG. 57.

FIG. 59 is a perspective view of a device having a Scotch yoke mechanism, according to an embodiment of the present disclosure.

FIGS. 60A-60B are side views of the operation of a device having an exemplary driving wedge mechanism, according to an embodiment of the present disclosure.

FIGS. 61A-61D are side views of the operation of a device having an exemplary driving wedge mechanism, according to an embodiment of the present disclosure.

FIG. 62 is a perspective view of the device of FIGS. 61A-61D, according to an embodiment of the present disclosure.

FIG. 63 is a perspective view of a device having an exemplary mechanism employing pin-in-slot operation, according to an embodiment of the present disclosure.

FIG. 64 is a side view of the device of FIG. 63.

FIG. 65 is a perspective view of a device having an exemplary single rack-and-pinion mechanism, according to an embodiment of the present disclosure.

FIG. 66 is a front view of a human skeleton with certain individual bones indicated.

FIG. 67A is a side view of a parallel (transverse) translation mechanism to move a bone for cortex distraction or to move a plate for periosteal distraction, according to an embodiment, to the present disclosure.

FIG. 67B is a side view of an angular translation mechanism to move a bone for cortex distraction or to move a plate for periosteal distraction, according to an embodiment, to the present disclosure.

FIG. 67C is a side view of parallel plane rotation mechanism to move a bone for cortex distraction or to move a plate for periosteal distraction, according to an embodiment, to the present disclosure.

FIG. 67D is a side view of an angular distraction mechanism to move a bone for cortex distraction or to move a plate for periosteal distraction, according to an embodiment, to the present disclosure.

FIG. 67E is a side view of an opening or closing book mechanism to move a bone for cortex distraction or to move a plate for periosteal distraction, according to an embodiment, to the present disclosure.

FIG. 67F is an elevation view of a bone shuttle mechanism to move a bone for cortex distraction or to move a plate for periosteal distraction, according to an embodiment, to the present disclosure.

FIG. 67G is an elevation view of a rotation mechanism around a center axis to move a bone for cortex distraction or to move a plate for periosteal distraction, according to an embodiment, to the present disclosure.

FIG. 67H is a perspective view of a rotation mechanism along a longitudinal axis to move a bone for cortex distraction or to move a plate for periosteal distraction, according to an embodiment, to the present disclosure.

FIGS. 671-67K are elevation views of a method of fusing bone and then lengthening.

FIG. 68A is a sectional view, with a detailed inset, of a fixation of a bone segment utilizing a threaded anchor, according to an embodiment of the present disclosure.

FIG. 68B is a sectional view, with a detailed inset, of a fixation of a bone segment utilizing a suture, according to an embodiment of the present disclosure.

FIG. 68C is a sectional view, with a detailed inset, of a fixation of a bone segment utilizing a staple, according to an embodiment of the present disclosure.

FIG. 68D is a sectional view, with a detailed inset, of a fixation of a bone segment utilizing wire with beads, according to an embodiment of the present disclosure.

FIG. 68E is a sectional view, with a detailed inset, of a fixation of a bone segment utilizing a pins, according to an embodiment of the present disclosure.

FIG. 68F is a sectional view, with a detailed inset, of a fixation of a bone segment utilizing a pins, according to an embodiment of the present disclosure.

FIG. 69A is a perspective view of a distraction mechanism utilizing shape memory wires, according to an embodiment of the present disclosure.

FIGS. 69B-69C are side views of an alternative embodiment of the distraction mechanism of FIG. 69A utilizing a clutch, according to an embodiment of the present disclosure.

FIG. 70 is a cross-section view of a distraction device utilizing an electric gearmotor, according to an embodiment of the present disclosure.

FIG. 71 is a perspective view of an external fixation device utilizing pulleys, according to an embodiment of the present disclosure.

FIG. 72 is a perspective view of an external fixation device utilizing a cable spool, according to an embodiment of the present disclosure.

FIG. 73 is a perspective view of an external fixation device utilizing ring attachment of a distraction device, according to an embodiment of the present disclosure.

FIG. 74 is a perspective view of an external fixation device utilizing a translation leadscrew, according to an embodiment of the present disclosure.

FIG. 75 is a perspective view of an external fixation device utilizing a rack and pinion drive, according to an embodiment of the present disclosure.

FIG. 76A is a side view of a clamp for use with an external fixation device, according to an embodiment of the present disclosure.

FIG. 76B is an end view of the clamp of FIG. 76A.

FIG. 77 is a side view of a distraction device for bolting onto a strut of an external fixation device, according to an embodiment of the present disclosure.

FIG. 78 is a perspective view of an external fixation device with a strut configured to couple to a distraction device, according to an embodiment of the present disclosure.

FIG. 79 is a perspective view of an external fixation device with a strut having a built-in clamp, according to an embodiment of the present disclosure.

FIG. 80 is a perspective view of a wirelessly-powered periosteal distraction device, according to an embodiment of the present disclosure.

FIG. 81 is a perspective view of a wirelessly controlled distraction device implanted on a bone, according to an embodiment of the present disclosure.

FIG. 82 is an elevation view of a first step for implanting a distraction device, according to an embodiment of the present disclosure.

FIG. 83 is an elevation view of a second step utilizing a drill guide, according to an embodiment of the present disclosure.

FIG. 84 is a side view of a third step for preparing a bone, according to an embodiment of the present disclosure.

FIG. 85 is a side view of a fourth step placement of a wirelessly controlled distraction device, according to an embodiment of the present disclosure.

FIG. 86 is a side view of the wirelessly controlled distraction device implanted in a patient, according to an embodiment of the present disclosure.

FIG. 87 is plan view of the operation of the wirelessly controlled distraction device, according to an embodiment of the present disclosure.

FIG. 88 is a cross-sectional view of a distraction device, according to an embodiment of the present disclosure.

FIG. 89 is a top view of a drill/cutting guide for use with the distraction device of FIG. 88, according to an embodiment of the present disclosure.

FIG. 90 is a cross-sectional view of a manual distraction device comprising a support frame attached to a bone, according to an embodiment of the present disclosure.

FIG. 91 is a top view of the support frame of FIG. 90.

FIG. 92 is a cross-section view of the support frame of FIG. 90.

FIG. 93 is a side view of a distraction device, according to an embodiment of the present disclosure.

FIG. 94 is a side view of a distraction device, according to an embodiment of the present disclosure.

FIG. 95 is a side view of a distraction device, according to an embodiment of the present disclosure.

FIG. 96 is a side view of a distraction device, according to an embodiment of the present disclosure.

FIG. 97 is an exploded perspective view of a distraction device utilizing a rack and pinion mechanism, according to an embodiment of the present disclosure.

FIG. 98 is a perspective view of a first trans-cutaneous magnetic drive, according to an embodiment of the present disclosure.

FIG. 99 is a sectional view of a second trans-cutaneous magnetic drive, according to an embodiment of the present disclosure.

FIG. 100 is a perspective view of a first trans-cutaneous magnetic drive, according to an embodiment of the present disclosure.

FIGS. 101A-101E are simplified representations of the operation of a two-pole actuator for rotating a radially-poled magnet, according to an embodiment of the present disclosure.

FIGS. 102A-102E are simplified representations of the operation of a two-pole actuator for rotating a radially-poled magnet, according to an embodiment of the present disclosure.

FIG. 103 is a perspective view of a trans-cutaneous magnetic charging system, according to an embodiment of the present disclosure.

FIG. 104 is a perspective view of a distraction mechanism utilizing shape memory wires, according to an embodiment of the present disclosure.

FIG. 105 is a cross-sectional view of a locking screw secured to a portion of a distraction device, according to an embodiment of the present disclosure.

FIG. 106 is a perspective exploded view of a collet cap fixation assembly, according to an embodiment of the present disclosure.

FIG. 107 is a sectional view of the collet cap fixation assembly of FIG. 107.

### DETAILED DESCRIPTION OF THE ILLUSTRATED EMBODIMENTS

Distraction osteogenesis (DO) or distraction histogenesis (DH), provides a method for repairing complex bone fractures using an external or internal fixation apparatus that applies a slow, steady, continuous distraction to living tissue to stimulate local tissue regeneration and active growth. Periosteal distraction osteogenesis (PDO) or periosteal distraction histogenesis (PDH) use the osteogenicity of periosteum, which creates an artificial space between the bone surface and periosteum to generate new bone by gradually expanding the periosteum. This can be done without requiring any corticotomy. Periosteal distraction osteogenesis (PDO) or periosteal distraction histogenesis (PDH) can induce new bone formation, and can also effectively increase blood flow, guide tissue generation, and augment soft tissue. Periosteal distraction (PD) is a broad name for the technology and medical technique.

Soft tissue pressure injuries are a major health problem with high morbidity, high burden to healthcare, and increased mortality rates. The inventors have recognized that the distraction of the tibial cortex and stretching of the periosteum each via transverse tibial transport (TTT) and periosteal distraction (PD) each result in an increase of regenerative growth factors and stem cell serum levels, with positive effects observed on angiogenesis and blood flow on the non-treated limb. These techniques can also raise systemic growth factor levels. They can also increase perfusion and microvascularization. The inventors have developed devices and procedures that provide safer, less and/or non-invasive procedures with improved management during transverse tibial cortex transport (TTT) treatment to make available effective therapies to alleviate the suffering of patients having a wide range of medical conditions arising from circulation impairment and/or bone injury/deformity.

The inventors have also developed apparatus and methods for making and using various devices for moving bone materials and related materials at a variety of sites in the body. The applications include many in which growth such as angiogenesis or nerve generation is desirable and can be achieved. A large number of patients can benefit from such apparatus and techniques, including patients having diabetes. Effective therapies are thus made possible to alleviate the suffering of patients having a wide range of medical conditions arising from circulation impairment and/or bone injury/deformity.

In some embodiments, a device is similar to a trauma plate, but has actuating members that can controllably (automatically or semi automatically) move a bone fragment or periosteum and induce distraction osteogenesis or distraction histogenesis, either invasively or non-invasively. The fixed portion of the device allows for fixation into the bone using anchors, such as bone anchors like fixation pins or locking screws, while the articulating member of the device also uses locking screws to provide fixation to the intercalary segment in order to control its movement. The embodiments of the device allow the TTT or PD fixation construct to be located internally. The technique need not use a tibia as the main bone, or a piece of tibial bone. Other bones can be utilized, including, but not limited to: a mandible, a clavicle, a humerus, a radius, an ulna, a femur, a fibula, an ilium, a phalange, a tibia, and a sacrum.

The implant has an internal actuator and drive mechanism to move the articulating members of the device. The cortex distraction element moves perpendicularly away from the fixed main body of the device. In some embodiments, in addition to perpendicular movement, a hinge-type opening angle type of actuation can also achieve distraction osteogenesis or distraction histogenesis. The member is controlled by rotation of output shaft of the actuator or through the gears on the shaft.

The torque output from the motor, or magnet, can be multiplied using an offset flat spur gear chain system, which allows the resulting amplified output torque to rotate a leadscrew or ball screw and nut element to allow a threaded mechanical interaction that converts rotational motion to linear movement. The torque output from the motor, or magnet, can be multiplied with worm gearing (worm drive), for example a worm engaging a worm wheel.

While useful for patient needs involving application of a device to the tibia for diabetic foot ulcer treatment, the inventive devices and techniques can also be used for any bone on the body, as the scientific principles of distraction osteogenesis or distraction histogenesis apply for all bones in the body. The inventors have recognized that it is possible that the distraction of the tibial cortex results in an increase of regenerative growth factors and stem cell serum levels, with positive effects observed on angiogenesis and blood flow on the non-treated limb. This technique can raise systemic growth factor levels, which may also be experienced in proximity of the distraction osteogenesis or distraction histogenesis site. Embodiments of the devices described herein can be coupled to many different bones in the body, because the devices are internal and implantable, allowing targeted treatment of soft tissue injuries in virtually any area of the body. For example, a patient presenting with sacral pressure ulcers could receive this technique and device in the pelvis which could localize the accelerated biologic activity.

The utility of the devices and techniques described herein is not necessarily limited to pressure ulcers but could be used to treat any soft tissue like primary lymphedema or injuries, including burns or acute traumatic injuries which would normally require the use of skin grafts. The use of this device could also be used for the healing of vascular or nerve grafts. Further, it should be noted that while many examples described herein specifically refer to TTT, i.e., transverse tibial cortex transport, PD, i.e., periosteal distraction, the inventive devices and methods are applicable to a broad range of other therapies, including but not limited to, horizontal ipsilateral bone transport for treating segmental bone defects.

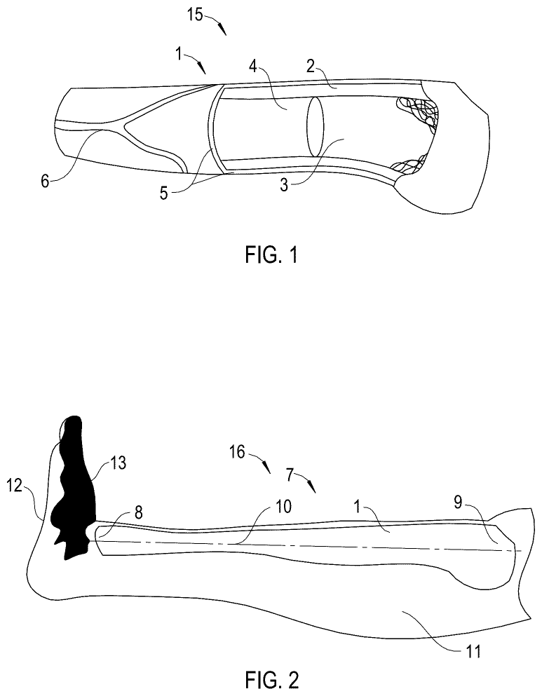

FIG. 1 illustrates a partially dissected tibia portion 1 of a patient 15 comprising an outer cortex 2 comprising hard, cortical bone and a medullary cavity 3. The anatomy of the tibia 1 includes many characteristics that can be found in a range of bones in the human body. Marrow 4 fills the medullary cavity 3. A periosteum 5, is a tissue layer that adheres to an outer surface of the tibia 1, as it does in most bones in the body. A network of blood vessels 6 include arteries that supply blood to the tibia 1 including the medullary cavity 3 and veins that provide a pathway for exiting blood. Many of these blood vessels 6 are not shown in FIG. 1 because they are in the cut-away portion.

FIG. 2 illustrates a lower leg 7 of a diabetic patient 16, demonstrating that the tibia 1 comprises a distal end 8 and a proximal end 9 and generally extends along a longitudinal axis 10. The calf 11 area and foot 12 are also shown, the foot 12 in this particular patient 61 having a diabetic foot ulcer 13. Foot ulcers 13 are found in approximately 15% of diabetic patients. Diabetic foot ulcers 13 are the most common cause of lower extremity amputations that are not caused by trauma, with amputation being required in between 14% to 24% of patients having diabetic foot ulcers 13. Treatment in the related bones of diabetic patients having foot ulcers, or in the process of developing foot ulcers, or other similar ulcers, can be initiated with one or more of the embodiments presented herein. Diabetic foot ulcers are only one type of ischemic foot ulcers. The embodiments described herein are all configured to treating all types of ischemic foot ulcers, not only those caused and/or exacerbated by diabetes. Ischemic foot ulcers can also be caused by one or more of: atherosclerosis, arteriosclerosis, peripheral artery disease (PAD) caused by hyperlipidemia, hypertension, smoking, chronic kidney disease, lupus, and even badly-healed trauma with or without underlying conditions like alcoholism, bad stress management, cancer, concurrent radiation therapy, or autoimmune diseases.

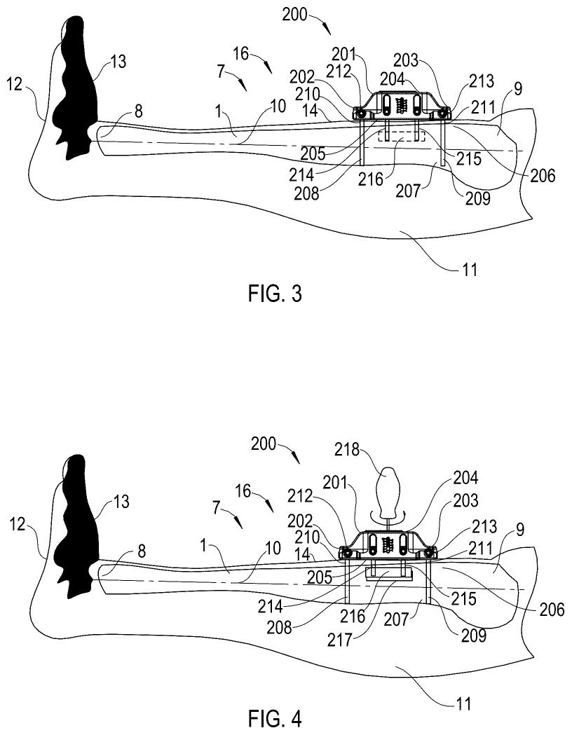

FIGS. 3-4 illustrate a transverse tibia transport device (TTT device) 200 coupled to a tibia 1 of a patient 16 having a foot ulcer 13. The TTT device 200 comprises a base 201 having a first end 202, and second end 203, an upper surface 204, and a lower surface 205. The upper surface 204 and the lower surface 205 each extend between the first end 202 and the second end 203. In FIGS. 3-4, the base 201 has been coupled such that it is external to the skin 14 surrounding the lower leg 7, with the first end 202 pointing toward the distal end 8 of the tibia 1 and the second end 203 pointing toward the proximal end 9 of the tibia 1.

The base 201 is statically coupled to cortical bone at a first side 206 of the tibia 1 and a second side 207 of the tibia 1 with bone anchors 208, 209, which can comprise bone screws. The bone anchors 208, 209, at first ends 210, 211, are statically coupled to the base 201 by securing the first ends 210, 211 with set screws 212, 213, respectively. In a first type of setup, the base 201 can be coupled to the tibia 1 via the bone anchors 208, 209 such that the lower surface 205 of the base 201 contacts and rests against the skin 14. In second type of setup, a protective material, such as cotton gauze (not shown) is sandwiched between the lower surface 205 of the base 201 and the skin 14. In a third type of setup, a space is maintained between the lower surface 205 of the base 201 and the skin 14. The space can be between 0.05 mm and 30 mm, or between 1 mm and 20 mm, or between 2 mm and 10 mm.

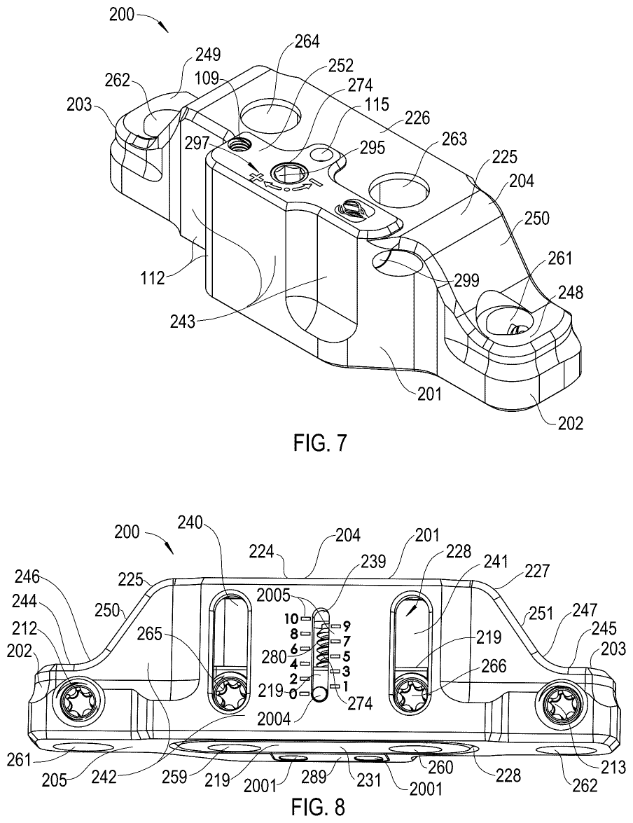

A movable stage 219 (FIGS. 8-9 and 14-16) will be described in more detail. The movable stage 219 is internal to the base 201 and movable relative to the base 201 via a rotatable leadscrew 274 (FIGS. 7-9 and 13-16) that is longitudinally constrained within the base 201. In some embodiments, substantially all of the leadscrew 274 is longitudinally maintained between the upper surface 204 and the lower surface 205 of the base 201. The movable stage 219 is statically coupled to two dynamic (movable) bone anchors 214, 215. A piece 216 of the tibia 1 is cut from the tibia 1 such that it can be moved independently of the tibia, e.g., underneath the periosteum 5. The piece 216 of the tibia 1 can comprise a number of shapes, including a rectangle, a square, a circle, an oval, an ellipse, a triangle, or other shapes. The separation of the piece 216 from the remainder of the tibia 1 leaves an open space 217 in the tibia 1. The open space 217 is surrounded by one or more cut walls of the tibia 1 and forms a shape including, but not limited to, including a rectangle, a square, a circle, an oval, an ellipse, or a triangle. The dynamic bone anchors 214, 215 are each statically coupled to the piece 216 of the tibia. The bone anchors 208, 209 and the dynamic bone anchors 214, 215 pass through the skin 14 of the patient 16. Access through the skin can be created by the user via a number of surgical methods, such as incision or puncture, with one or more surgical tools, or with the bone anchors 208, 209, 214, 215, themselves. Using a torque tool, such as a screwdriver 218, a head of the leadscrew 274 is engaged and rotated in a first rotational direction (arrow, FIG. 4) causing the stage 219 to move relative to the base 201 and in turn translating the dynamic bone anchors 214, 215 that are attached to the piece 216 that was cut from the tibia 1. By movement of this piece 216 of the tibia 1 in a direction that includes a transverse component in relation to the longitudinal axis 10 of the tibia 1, the growth-related stimulation can occur, including an increase of regenerative growth factors and systemic growth factors, stem cell serum levels, angiogenesis, and blood flow on the treated lower leg, and systemically. Though two bone anchors 208, 209 and two dynamic bone anchors 214, 215 are represented, other embodiments utilizing only one bone anchor 208 and/or only one dynamic bone anchor 214 are also possible. By having sufficient diameter or transverse dimension (if not round in cross-section) for reduced bending, a single anchor 208, 214 can stabilize the base 201 in relation to the tibia 1 and stabilize the bone portion 216 in relation to the movable stage 219.

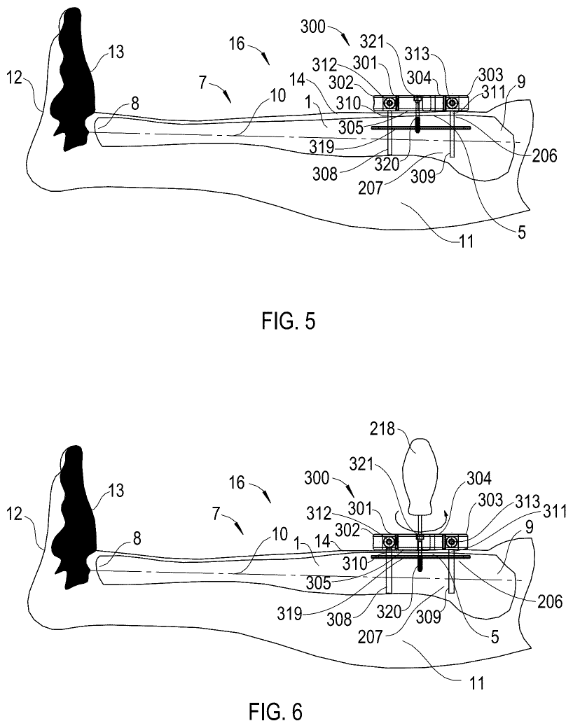

FIGS. 5-6 illustrate a periosteal distraction device (PD device) 300, or periosteal stretching device, coupled to a tibia 1 of a patient 16 having a foot ulcer 13. The PD device 300 comprises a base 301 having a first end 302, and second end 303, an upper surface 304, and a lower surface 305. The upper surface 304 and the lower surface 305 each extend between the first end 302 and the second end 303. In FIGS. 5-6, the base 301 has been coupled such that is external to the skin 14 surrounding the lower leg 7, with the first end 302 pointing toward the distal end 8 of the tibia 1 and the second end 303 pointing toward the proximal end 9 of the tibia 1. The base 301 comprises anodized aluminum, or another high-strength material.

The base 301 is statically coupled to cortical bone at a first side 206 of the tibia 1 and a second side 207 of the tibia 1 with bone anchors 308, 309, which can comprise bone screws. The bone anchors 308, 309, are statically coupled to the base 301 by securing the bone anchors 308, 309 with set screws 312, 313, respectively. In a first type of setup, the base 301 can be coupled to the tibia 1 via the bone anchors 308, 309 such that the lower surface 305 of the base 301 contacts and rests against the skin 14. In a second type of setup, a protective material, such as cotton gauze (not shown) is sandwiched between the lower surface 305 of the base 301 and the skin 14. In a third type of setup, a space is maintained between the lower surface 305 of the base 301 and the skin 14. The space can be between 0.05 mm and 30 mm, or between 1 mm and 20 mm, or between 2 mm and 10 mm.

A movable stage 319, comprising a plate, movable relative to the base 301 via a rotatable leadscrew 320 that is longitudinally constrained within the base 301. The movable stage 319 is implanted beneath a section of periosteum 5 of the patient 16. The movable stage 319 includes two holes 322, 323 configured to allow the bone anchors 308, 309 to pass through, with clearance, thus allowing the movable stage 319 to translate in relation to the bone anchors 308, 309. The bone anchors 308, 309 and the leadscrew 320 pass through the skin 14 of the patient 16. Using a torque tool, such as a screwdriver 218, a head 321 of the leadscrew 320 is engaged and rotated in a first rotational direction (arrow, FIG. 6) causing the stage 319 to move relative to the base 301 and in turn moving, distracting, and/or stretching the periosteum 5 away from the tibia 1 (e.g., applying tension to the periosteum from above, or via a compressive force applied from below). The movable stage 319 can be referred to as a sub-periosteal member. By rotating the leadscrew 320 in the opposite rotational direction (opposite the arrow in FIG. 6), the stress applied to the periosteum 5 is reduced. By movement of the periosteum 5 of the tibia 1 in a direction that includes a transverse component in relation to the longitudinal axis 10 of the tibia 1, the growth-related stimulation can occur, including an increase of regenerative growth factors and systemic growth factors, stem cell serum levels, angiogenesis, and blood flow on the treated lower leg, and systemically. In some embodiments, the leadscrew 320 and the movable stage each comprise stainless steel. In some embodiments, the movable stage is a length of between about 30 mm and about 100 mm, or between about 40 mm and about 80 mm, or between about 30 mm and about 50 mm, or between about 60 mm and about 100 mm, or between about 70 mm and about 90 mm, or about 80 mm. In some embodiments, the movable stage is a width of between about 5 mm and about 20 mm, or between about 8 mm and about 15 mm, or between about 8 mm and about 12 mm, or between about 9 mm and about 11 mm, or about 10 mm. In some embodiments, the movable stage 319 is substantially circle-shaped, and has a diameter of between 1 mm and 100 mm, or between 5 mm and 50 mm. In some schemes, the periosteum is distracted at a displacement per time rate of about 1 mm per day. This can comprise, for example, 0.25 mm four times a day, or 0.1 mm ten times a day. A total amount of distraction of the periosteum can comprise between 2 mm and 20 mm or 4 mm to 15 mm, or 5 mm to 10 mm. The rate of distraction can vary, for example, from about 0.5 mm per day to about 1.0 mm per day. The total amount of distraction per day can be broken up into any number of sub-distractions, and can in some embodiments even comprise continuous distraction (non-stop, slow).

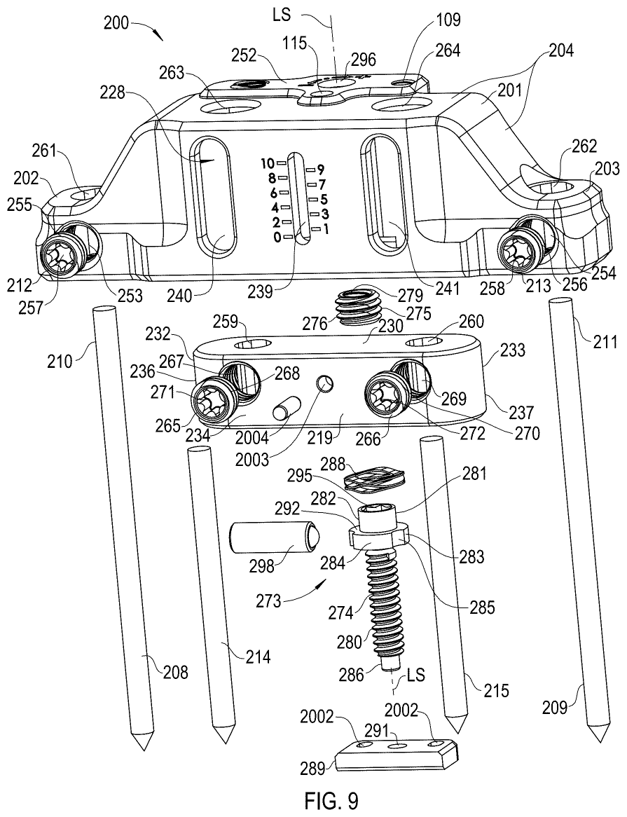

FIGS. 7-12 illustrate details of the transverse tibia transport device (TTT device) 200. The base 201 comprises a first end 202, and second end 203, an upper surface 204, and a lower surface 205. The upper surface 204 and the lower surface 205 each extend between the first end 202 and the second end 203. The base 201 includes a central extended portion 224 having a substantially planar top area 226 of the upper surface 204, extending between a first fillet 225 and a second fillet 227 of the upper surface 204. The central extended portion 224 comprises a central translation cavity 228 having a generally oval shape. In other embodiments, the central translation cavity 228 can have a shape or cross-section that is rectangular, square, or circular. The overall three-dimensional space of the cavity 228 can be box-shaped, or include filleted sides, edges, or ends. A movable stage 219 has a substantially matching oval shape with the translation cavity 228, but with slightly smaller dimensions, and is configured to translate, with clearance, up and down within the translation cavity 228. The movable stage 219 comprises an upper surface 230 (FIG. 9), a lower surface 231 (FIG. 8), a first end 232, a second end 233, a front face 234, and a back face 235. The first end 232 comprises a first semi-cylindric face 236, and the second end 233 comprises a second semi-cylindric face 237. The translation cavity 228 is bounded by a planar front wall 238 (FIG. 11) having a central, vertically-extending window 239 (FIG. 8) and two vertically-extending access apertures 240, 241, one on each side of the vertically-extending window 239.

The base 201 further comprises a front face 242, a back face 243, a first reduced-thickness end portion 244, and a second reduced-thickness end portion 245. A first curvilinear transition portion 246 extends between a first upper portion 248 of the upper surface 204 and an angled portion 250 of the upper surface 204. A second curvilinear transition portion 247 extends between a second upper portion 249 of the upper surface 204 and an angled portion 251 of the upper surface 204. The upper surface 204 further comprises a raised access area 252. Bone anchors 208, 209, at first ends 210, 211, are statically coupled to the base 201 by securing the first ends 210, 211 with set screws 212, 213, respectively. The base 201 comprises a first female-threaded hole 253 configured to threadingly engage a male thread 255 of the first set screw 212, and a second female-threaded hole 254 configured to threadingly engage a male thread 256 of the second set screw 213. The set screws 212, 213 each comprise an engagement tip (not shown) for frictionally engaging the bone anchors 208, 209, and a screw drive 257, 258 for keyingly engaging a drive tip of a torque tool, such as a screwdriver or a motorized driver. The screw drives 257, 258 can comprise any non-circular cavity shape, but can alternatively comprise a non-circular protrusion/protuberance. The non-circular shape of the screw drives 257, 258 can comprise any one of a hex, a torx, a slot, a Phillips or other X-shape or cruciform shape, a Robertson or other square shape, a security or tamper-resistant shape, an oval, a spanner, a pentalobular, a tri-point, a multiple square, or any combination thereof.

The first reduced-thickness end portion 244 includes a vertically-extending hole 261 passing therethrough, and the second reduced-thickness end portion 245 includes a vertically-extending hole 262 passing therethrough. The holes 261, 262 are sized to allow the bone anchors 208, 209 to be passed therethrough. In some embodiments, the holes 261, 262 can comprise counter-bores to accommodate screw heads of bone screws. The movable stage 219 comprises a first vertically-extending hole 259 and a second vertically-extending hole 260, passing therethrough. The holes 261, 262 are sized to allow the bone anchors 208, 209 to be passed therethrough. The holes 259, 260 of the movable stage 219 are sized to allow two dynamic (movable) bone anchors 214, 215 to be passed therethrough. The movable stage 219 comprises a first female-threaded hole 267 configured to threadingly engage a male thread 268 of a first set screw 265, and a second female-threaded hole 269 configured to threadingly engage a male thread 270 of a second set screw 266. The set screws 265, 266 each comprise an engagement tip (not shown) for frictionally engaging the dynamic bone anchors 214, 215, and a screw drive 271, 272 for keyingly engaging a drive tip of a torque tool, such as a screwdriver or a motorized driver. The screw drives 271, 272 can comprise any non-circular cavity shape, but can alternatively comprise a non-circular protrusion/protuberance. The non-circular shape of the screw drives 271, 272 can comprise any one of a hex, a torx, a slot, a Phillips or other X-shape or cruciform shape, a Robertson or other square shape, a security or tamper-resistant shape, an oval, a spanner, a pentalobular, a tri-point, a multiple square, or any combination thereof. There are two additional holes 263, 264 at the central extended portion 224 of the base 201 that are configured to allow the dynamic bone anchors 214, 215 to translate freely therein. In some embodiments, the holes 259, 260 can comprise internal (female) threads, to allow threaded engagement with the male thread of a bone screw.

In use, a user drives the bone anchors 208, 209 into the desired bone (e.g., tibia) for static engagement, and drives the dynamic bone anchors 214, 215 into a bone piece 216 or other growth stimulator for static engagement. With the movable stage 219 located within the translation cavity 228 of the base 201, the bone anchors 208, 209 inserted through the holes, and the user secures the bone anchors 208, 209 to the base 201 by tightening the set screws 212, 213 against the bone anchors 208, 209, to frictionally hold them in relation to the base 201. When placing the base 201 over the bone anchors 208, 209 with the movable stage 219 in place within the translation cavity 228, the dynamic anchors 214, 215 will have passed freely through the holes 259, 260 in the movable stage 219 and the holes 263, 264 of the base 201. The user then adjusts the height of the movable stage 219 within the translation cavity 228 of the now secured base 201, and then tightens the set screws 265, 266 against the dynamic bone anchors 214, 215, to frictionally hold them in relation to the movable stage 219.

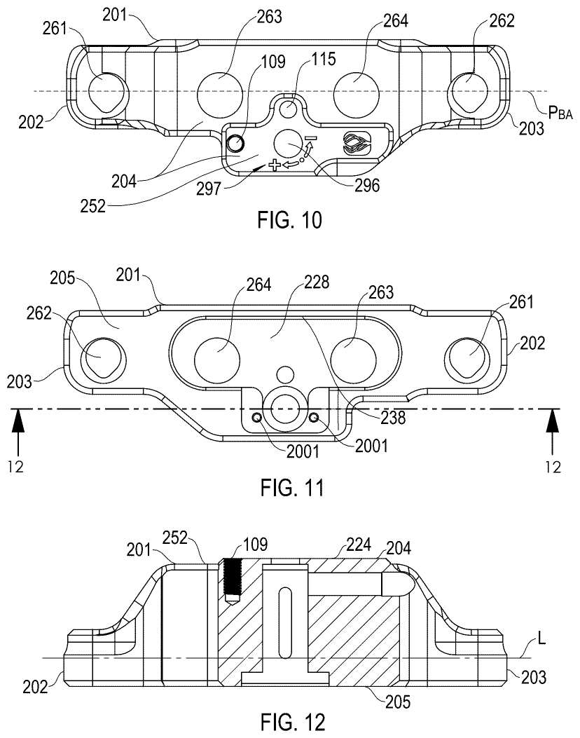

Internally, the base 201 and the movable stage 219 are coupled via a drive system 273 configured to enable translation of the movable stage 219 in relation to the base 201. The drive system 273 includes a leadscrew 274 rotatably contained within the base 201, and a nut 275 attached to the movable stage 219. The nut 275 has an external thread 276 and the movable stage 219 has an internally-threaded hole 277. The external thread 276 of the nut 275 is adhesively bonded to the internally-threaded hole 277 of the movable stage 219 such that the nut 275 and the movable stage 219 are statically coupled to each other. In other embodiments, the external thread 276 of the nut 275 is epoxy bonded or frictionally fit into the internally-threaded hole 277. The internally threaded hole 277 passes through a side projection 278 in the movable stage 219, such that the nut 275 and the leadscrew 274 have a leadscrew axis LS that is offset from a dynamic bone anchor plane PDBA (FIG. 15). In the embodiment of FIGS. 9-16, the dynamic bone anchor plane PDBA is substantially the same as a bone anchor plane PBA (FIG. 10). In other embodiments, the base 201 and the movable stage 219 can be configured such that the dynamic bone anchor plane PDBA is parallel to, but not the same as the bone anchor plane PBA. In other embodiments, the base 201 and the movable stage 219 can be configured such that the dynamic bone anchor plane PDBA at an angle to the bone anchor plane PBA. For example, the angle between can range from 1 degree to 60 degrees, or 5 degrees to 45 degrees, or 10 degrees to 30 degrees, wherein the two planes intersect at a line that is substantially parallel or colinear to base longitudinal axis L (FIG. 12).

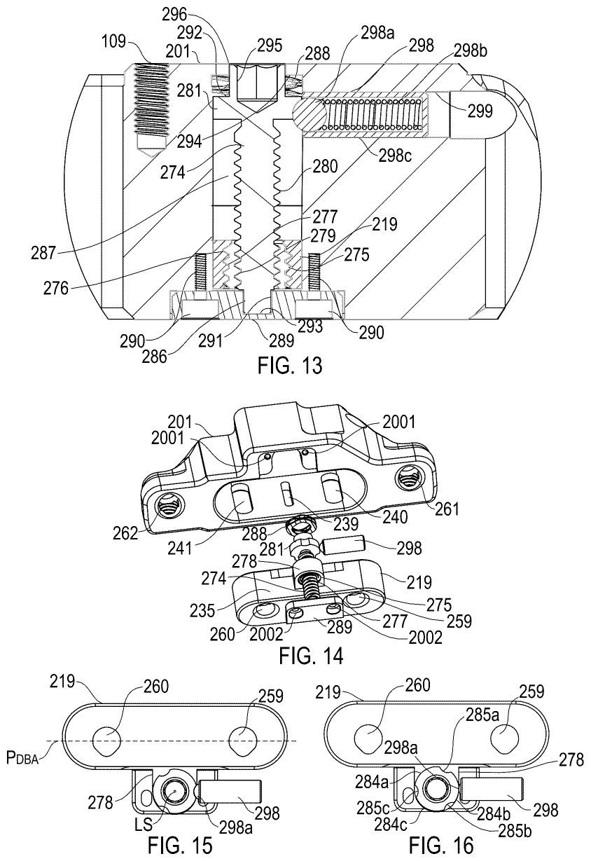

The nut 275 further comprises an internal thread 279 that is configured to threadingly engage an external thread 280 if the leadscrew 274. The leadscrew 274 further comprises a head 281 having a proximal flange 282 and a non-circular head section 283 between the flange 282 and the external thread 280. Turning to FIG. 16, the non-circular head section 283 comprises three convex arc portions 284a-c equally distributed around the leadscrew axis LS, and three concave depressions 285a-c, having arc shapes, equally distributed around the leadscrew axis LS, between each of the convex arc portions 284a-c. Instead of three of each, there can be one of each (e.g., wherein the arc of the single concave depression 285 is a lesser angle than the arc of the single convex arc portion, or vice versa). In some embodiments, there can be two of each or four or each or more. In some embodiments, the positions can even be asymmetrically distributed. The convex arc portions 284a-c and the concave depressions 285a-c together comprise an outer perimeter of the non-circular head section 383. The leadscrew 274 also comprises a distal cylindrical projection 286. The leadscrew 274 is captured within an internal leadscrew space 287 of the base 201 by placing three wave washers 288 over the proximal flange 282 and connecting a cap 289 to the base 201 with two screws 290 (FIG. 13). The two screws 290 pass through two holes 2002 in the cap 289, and then screw into two threaded holes 2001 in the base 201. The distal cylindrical projection 286 has a slightly smaller diameter than an internal cylindrical cavity 291 in the cap 289. The wave washers 288 apply a bias against an annular proximal face 292 of the non-circular head section 283 of the leadscrew 274 thus maintaining substantially longitudinal stability of the leadscrew 274 within the base 201, between an end face 293 of the internal cylindrical cavity 291 of the cap 289 and an annular face 294 of the base 201, against which the wave washers 288 apply their bi-directional bias. Although three wave washers 288 are utilized in the embodiment of FIG. 13, one or more wave washers can be used, or any other biasing member, such as a standard spring, or one or more a nylon or fluoropolymer washers or spacers. The head 281 of the leadscrew 274 includes an internal non-circular cavity 295 (screw drive) comprising a hex shape, though it can comprise any other non-circular shape. The cavity 295 is accessed (e.g, by a matching non-circular tip of the manual or motorized torquing tool) through an access hole 296 in the base 201. Markings 297 on the upper surface 204 of the base 201 indicate to a user the rotational directions for positive (+) and negative (−) relative translation of the movable stage 219 in relation to the base 201.

Using a torque tool, such as a screwdriver 218, a head 281 of the leadscrew 274 is engaged and rotated in a first rotational direction (arrow, FIG. 4) causing the movable stage 219 to move relative to the base 201 and in turn moving the dynamic bone anchors 214, 215 that are attached, for example, to the piece 216 that was cut from the tibia 1. By movement of this piece 216 of the tibia 1 in a direction that includes a transverse component in relation to the longitudinal axis 10 of the tibia 1, the growth-related stimulation can occur, including an increase of regenerative growth factors and systemic growth factors, stem cell serum levels, angiogenesis, and blood flow on the treated lower leg, and systemically.

A ball spring plunger 298 is statically held (e.g., frictional fit, adhesive, epoxy) within a horizontal cylindrical cavity 299 in the base 201, adjacent the non-circular head section 283 of the leadscrew 274, such that a ball 298a of the ball spring plunger 298 is configured to snap into and out of the concave depressions 285a-c as the leadscrew 274 is turned. The ball 298a has a bias into the concave depressions 285a-c that is applied by a spring 298b within a cylindrical shell 298c of the ball spring plunger 298. In FIG. 15, the leadscrew 274 is in a rotational orientation about the leadscrew axis LS such that one of the concave depressions 285 is engaged by the ball 298a of the ball spring plunger 298. In FIG. 16, the leadscrew 274 has been rotated to a rotational orientation about the leadscrew axis LS such that none of the concave depressions 285a-c is aligned with the ball 298a of the ball spring plunger 298. Thus, the ball 298a is forced by a convex arc portion 284 to compress the spring 298b into the shell 298c of the ball spring plunger 298. Each of the convex arc portions 284a-c, or other similar positive-space or neutral-space feature, comprises at least some radial projection or protrusion in relation to the concave depressions 285a-c, transverse to the leadscrew axis LS. Each of the concave depressions 285a-c, or other similar negative-space engagement feature, comprises at least some radial indentation in relation to the convex arc portions 284a-c, transverse to the leadscrew axis LS. A physical reaction occurs between a concave depression 285 and the ball 298a of the ball spring plunger 298 when the leadscrew 274 is moved from a non-engaged position (ball 298a compressed by convex arc portion 284) to an engaged position (ball 298a allowed to release into concave depression 285). This physical reaction (e.g., of a first metal piece accelerating into and striking another metal piece) produces a physical disturbance configured to be sensed by the user as an audible sound and/or as a tactile vibration. This disturbance provides information to the user, via the user's ears and/or fingers/hands/arm or other body part or sense, and it relates to the user the extent of relative displacement between the translatable anchor and the base. FIGS. 9-16 illustrate an embodiment having three concave depressions 285a-c, separated from each other by about 120° around the head 281. In other embodiments, there can be only a single concave depression 285, such that each 360° rotation has a single detent rotational orientation. In other embodiments, there can be only two concave depressions 285a-b, separated by about 180°. Depending on the precision desired, the concave depressions 285 can number more than three, for example four, five, six, seven, eight, nine, ten, or more than ten. Although they are generally evenly distributed around the head 281, in other embodiment, the distribution can be uneven. For example, two concave depressions 285, 30° apart and then two more depressions 30° apart at the opposite side of the head 281.

In a first embodiment, for example, each “click” sound that is heard by the user's ear, can represent one-third of a millimeter of movement of the movable stage 219 away from the tibia 1. In a second embodiment, each strong pulse of vibration that is felt by the user's hand can represent one-half millimeter of movement of the movable stage 219 toward the tibia 1. The audible sound can comprise a sound pressure level between 20 dB and 80 dB at a distance of 0.9 meter, or between 20 dB and 70 dB at a distance of 0.9 meter, or between 20 dB and 60 dB at a distance of 0.9 meter.

The front face 234 of the movable stage 219 includes a transverse hole 2003 into which a dowel pin 2004 is press fit. The pin 2004 extends such that it travels up and down within the vertically-extending window 239 in the front face 242 of the base 201. The front face 242 of the base 201 includes indicators 2005 adjacent the vertically-extending window 239. Thus, the location of the pin 2004 in relation to the indicators 2005 allows a user to visualize the current amount of displacement of the movable stage 219 in relation to the base 201, and thus, in use, the amount of displacement of the piece 216 cut from the tibia 1, in relation to the tibia 1, itself. In other embodiments, the transverse hole 2003 and pin 2004 can be replaced by a visible mark on the movable stage 219 that can be seen within the vertically-extending window 239. In some embodiments, the visible mark is a bright color. In some embodiments, the visible mark can even comprise an LED. The bone piece 216 can be cut or otherwise removed from any bone, not necessarily the bone to which the anchors 208, 209 are coupled. These include, but are not limited to: a mandible, a clavicle, a humerus, a radius, an ulna, a femur, a fibula, an ilium, a phalange, a tibia, and a sacrum. Alternatively, instead of the bone piece 216, a non-bone growth stimulator can be utilized, including, but not limited to: a ceramic material, a glass material, a metal material, and a polymeric material. The non-bone growth stimulator can comprise a lattice structure. One or more growth stimulating composition can be used with or instead of the growth stimulator or the bone piece 216, including, but not limited to: a growth factor, a drug, and an antibiotic. The growth stimulating composition can comprise a coating configured to coat the bone piece 216 or coat a non-bone growth stimulator. The growth stimulating composition can comprise a material configured to be eluted by the bone piece 216 or by a non-bone growth stimulator. The one or more growth stimulating composition for either the growth stimulator or the coating can be biologically-derived, or can be manufactured. The growth stimulator can comprise a plate or other element that is configured to engage the periosteum. The growth stimulator can also comprise the periosteum, itself, as it is stretched or otherwise deformed or moved. Any of the bone anchors 208, 209 and the dynamic anchors 214, 215 can comprise K-wires, Steinmann pins, half pins, or bone screws. In some embodiments, the bone anchors 208, 209 comprise a K-wire having a diameter of between 0.9 mm and 2.0 mm, or between 0.9 mm and 1.6 mm, or between 1.2 mm and 2.0 mm, or between 1.2 mm and 1.5 mm, or about 1.6 mm, or about 2.0 mm. The bone anchors are configured to be placed percutaneously.

Other features of the transverse tibia transport device (TTT device) 200 will be described in relation to the connectability of the TTT device 200 to a modular automated drive unit 102.

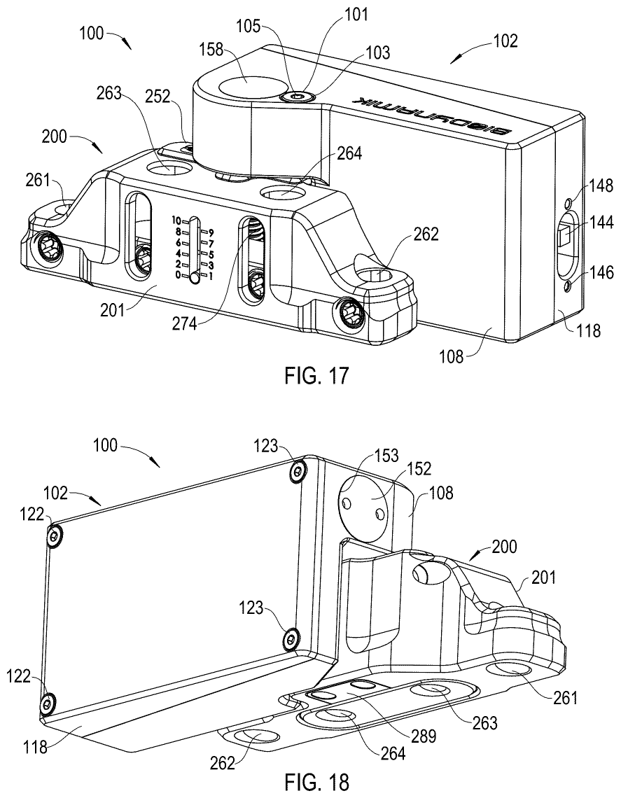

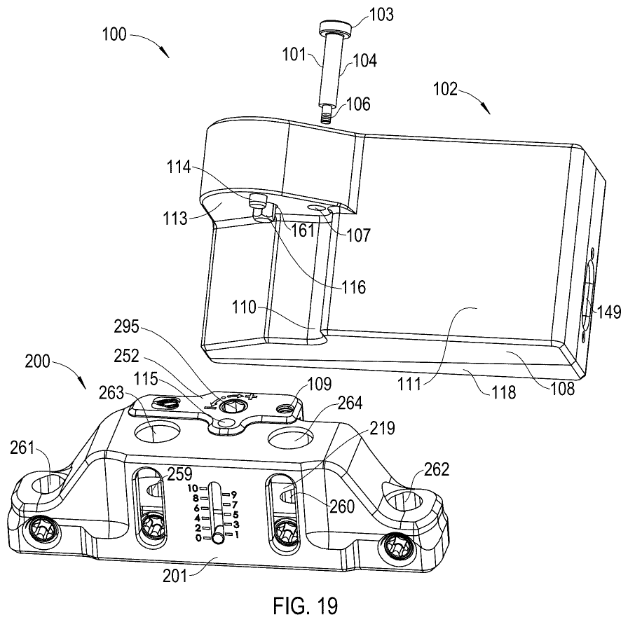

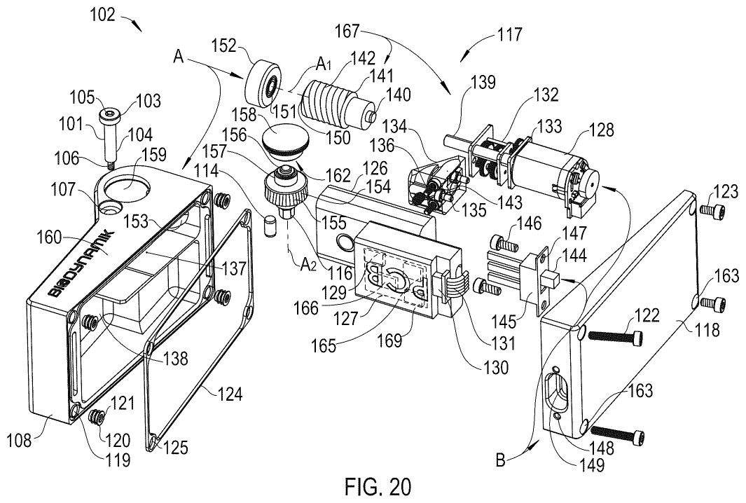

FIGS. 17-19 illustrate a system 100 for increasing biological activity within a patient comprising the transverse tibia transport device (TTT device) 200 and a modular automated drive unit 102. The modular drive unit 102 is configured to be coupled to the TTT device 200 for automatically driving the TTT device 200, and is configured to be decoupled from the TTT device 200. Turning to FIG. 19, a locking screw 101 comprises a head 103 having a screw drive 105, comprising a hexagonal cavity configured to be driven by a corresponding hex key or screwdriver tip. The locking screw 101 comprises a shaft 104 (or shank) and a male threaded tip 106. The shaft 104 has a diameter that closely fits through a hole 107 in a first housing half 108 (FIG. 20). When the transverse tibia transport device (TTT device) 200 is engaged with the modular drive unit 102, as will be described, the male threaded tip 106 of the locking screw 101 is threadingly engaged and tightened into a corresponding female threaded hole 109 in the base 101 of the TTT device 200. The housing 108/118 comprises the first housing half 108 and a second housing half 118.

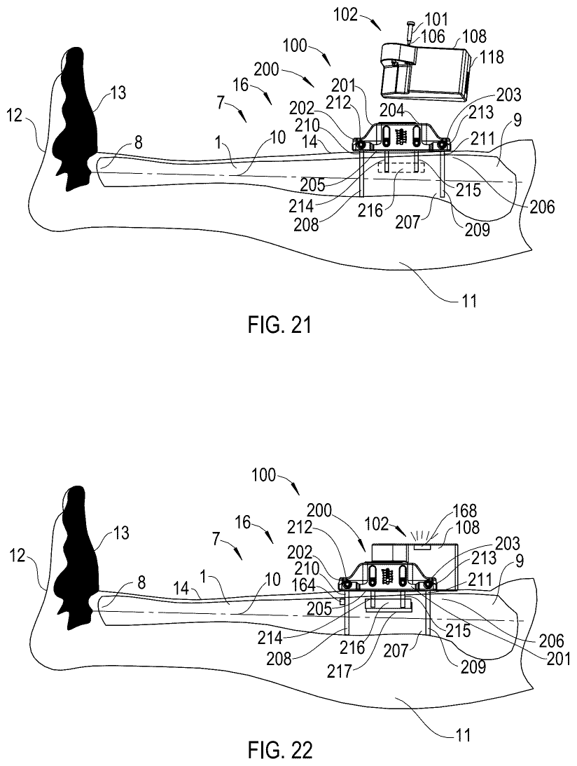

In use, a user can place the modular drive unit 102 onto a TTT device 200 prior to fully attaching the TTT device 200 to a patient. However, the following will be described in relation to a procedure in which the TTT device 200 is fully coupled to a patient 16, and subsequently the modular drive unit 102 is coupled to the TTT device 200 (see FIGS. 21-22). The TTT device 200 is engaged with the modular drive unit 102 by creating a substantially static fit between the TTT device 200 and the modular drive unit 102, and then locking the modular drive unit 102 to the TTT device 200 with the locking screw 101. A curved contour 110 on a front face 111 of the first housing half 108 of the drive unit 102 is configured to mechanically fit with a portion 112 of the back face 243 of the base 201 of the TTT device 200 (FIG. 7). Furthermore, a lower ledge face 113 of the first housing half 108 of the drive unit 102 is configured to rest on the raised access area 252 of the base 201 of the TTT device 200. A dowel pin 114 connected to the first housing half 108 and extending vertically downward therefrom is configured to insert into a hole 115 in the base 201 of the TTT device 200. The dowel pin 114 fits closely with the hole 115 and allows the modular drive unit 102 to index with the TTT device 200, although it does not need to be a friction fit. A driven output hex 116, which will be described in more detail, engages with and mates the non-circular cavity 295 of the leadscrew 274 of the TTT device 200. The driven output hex 116 turns within a clearance hole 161 of the first housing half 108. Thus, the curved contour 110, dowel pin 114 and driven output hex 116 of the modular drive unit 102 serve to fully index and stabilize the modular drive unit 102 with the TTT device 200, via their engagement with the portion 112, hole 115, and the non-circular cavity 295 of the TTT device 200. Because the dimensions of these features are maintained at standard levels of precision for molded plastic parts, it is relatively simple to place the nodular drive unit 102 onto a TTT device 200 that has been connected to a patient with sufficient engagement between the two. As mentioned, the locking screw 101 is then placed through the hole 107 in a first housing half 108 and the male threaded tip 106 is threadingly engaged with the female threaded hole 109 and the TTT device 200 and the modular drive unit 102 are locked together, by tightening of the locking screw 101 with a torque tool that has been inserted into the screw drive 105 of the locking screw 101. The curved contour 110 can be any type of chape that can hug or accept mating shape or fitting shape from the base 201 or any other portion of the TTT device 200. The engagement between the modular drive unit 102 and the TTT device 200 can be achieved with one or more element, including but not limited to: a pin, a hole, a depression comprising one or more linear boundaries, a depression comprising a curvilinear boundary, a protuberance comprising one or more linear boundaries, and a protuberance comprising one or more curvilinear boundaries. In some embodiments, one or more magnets can be used, such as permanent magnets or electromagnets, the magnets configured to be aligned with another magnet oriented such that opposite poles meet. In some embodiments one or more magnets can be used, and configured to be aligned with one or more ferrous metal piece, such as iron or steel.

Turning to FIG. 20, the modular drive unit 102 comprises components 117, including the first housing half 108 and a second housing half 118. The first housing half comprises four holes 119 into which externally-barbed, internally-threaded inserts 120 are press-fit, to provide internal threads 121. The two housing halves 108, 118 are secured together by long socket screws 122 and short socket screws 123, with a rectangular gasket 124 in between. The gasket 124 comprises four holes 125 that allow the screws 122, 123 to pass through. The screws 122, 123 are secured through holes 163 in the second housing half 118, holes 125 of the gasket 124, and into the internal threads 121. The gasket 124 allows the components 117 to be substantially sealed from the external environment. In FIG. 20, the components 117 are rotated approximately 90 degrees from the housing halves 108, 118 and gasket 124 in order to best illustrate all. End A and end B are labeled, for reference.

A battery 126 is configured to power circuitry 127 and an electric motor 128 The battery 126 can be a replaceable battery or a rechargeable battery. A rechargeable battery can be configured to be directly charged from a charging port, or wirelessly charged, via close inductive charging, or even by magnetic resonance wireless power transfer over distance. In some embodiments, the battery, itself, has a built-in charging circuit. In some embodiments, the battery 126 comprises a lithium or a lithium-ion polymer (LiPo) battery. The circuitry 127 comprises a printed circuit board PCB carrying a microcontroller 129. The microcontroller 129 can comprise a microprocessor. The microcontroller can have Bluetooth® (Bluetooth Special Interest Group) capability, and can comprise a built-in antenna. A connector 130 having conductors comprising cables or wires 131 is electrically coupled to the circuitry 127 and to the motor 128 (e.g., gearmotor) to provide power and control signals to the motor 128. The PCB can also couple to one or more port providing USB access and/or JTAG access. A switch 145 having a two or three position switch button 144 is coupled to an interior of the second housing half 118 by screws 146 that pass through clearance holes 147 in the switch 145, and secure to threaded holes 148 in the second housing half 118. The switch button 144 is accessible to a user through an access port 149 in the second housing half 118. In other embodiments, the switch 145 comprises a button that is pushed in to turn power on, but is twisted e.g., (spring-loaded, or along a threading) to turn off. For example, it can comprise an emergency power shut-off. The motor 128 is mechanically coupled to a gear box 132 at a connection site 133, which can comprise screw, other fasteners, or welding. The motor 128 and the gear box 132 are attached to the first housing half 108 by a motor mount 134. Two screws 135 attach the motor mount 134 to the gear box 132 and two screws 136 attach the motor mount 134 to two threaded holes 137 in an interior wall 138 of the first housing half 108. The gear box 132 includes an output shaft/connector 139 that secures to a projection 140 of a worm 141 having worm threading 142. The output shaft/connector 139 freely extends through a clearance hole 143 that passes through the motor mount 134. Thus, the output shaft/connector 139 is free to rotate.

The worm 141, at its distal end 150, couples to a ball bearing 151 that is held within an end cap 152 that inserts into a hole 153 in the first housing half 108, at side A. The ball bearing 151 allows the worm 141 to be stabilized and minimizes additional friction to the worm 141, as the worm 141 is rotated by the motor 128 and the gear box 132. The worm 141 transversely turns a worm wheel 154 having evenly cylindrically distributed teeth 155 configured to engage with the threading 142 of the worm 141. The driven output hex 116 is attached to the worm wheel via its shaft 156. The shaft 156 is held within a ball bearing 157 that is held within an internal circular pocket 162 in an end cap 158 that inserts into a hole 159 in a top side 160 of the first housing half 108. The combination of the gear box 132, the worm 141, and the worm wheel 154, and substantially everything between the motor 128 and the driven output hex 116 is referred to as the gear train 167.

Thus, rotation of the worm 141 in a first rotational direction about axis A1 causes rotation of the worm wheel 154 in a first rotational direction about axis A2. And, rotation of the worm 141 in a second, opposite rotational direction about axis A1 causes rotation of the worm wheel 154 in a second, opposite rotational direction about axis A2. Axis A1 is oriented substantially 90 degrees from A2. Thus, the length of the housing 108, 118 of the modular drive unit 102 between end A and end B can be parallel to the first end 202 and the second end 203 of the TTT device 200 as the lower ledge face 113 of the first housing half 108 of the drive unit 102 is placed in apposition to the raised access area 252 of the base 201 of the TTT device 200, and the elements 114, 116, 115, 295, 106, 109 are engaged and locked. And, the ball bearings 151, 157 maintain low friction rotation of the driven output hex 116, when it is coupled to the non-circular cavity 295 of the leadscrew 274 of the TTT device 200. In other embodiments, the worm 141 and worm wheel 154 can instead be replaced by an input bevel gear and an output bevel gear. Thus, a substantially 90-degree shaft angle (change in rotational axis) can also be accommodated, using the miter gear type of bevel gear combination. Other angles in between 90 degrees and zero degrees can also be accommodated with other bevel gear angles.

As in FIG. 4, FIG. 21 illustrates the TTT device 200 attached to the tibia 1 of a patient 16, as previously described. A user, or a patient 16 themself, decides to utilize a modular drive unit 102 to cause the movable stage 219 of the TTT device to move the bone piece 216 in relation to the tibia 1. The user places the modular drive unit 102 over and behind the TTT device 200 such that the curved contour 110 cradles the portion 112, the dowel pin 114 engages in the hole 115, and the driven output hex 116 keyingly fits inside the non-circular cavity 295. This indexes and stabilizes the modular drive unit 102 with the TTT device 200. The user then places the locking screw 101 through the hole 107 in a first housing half 108 and engages the male threaded tip 106 with the female threaded hole 109 and tightens the locking screw 101 with a torque tool that has been inserted into the screw drive 105. The modular drive unit 102 is now in place, coupled to the TTT device 200, as shown in FIG. 22. The user can now operate the modular drive unit 102 either from controls carried on the modular drive unit 102 not shown), or from a smart device/mobile device, or from an application on a cloud computing system. In some embodiments, the locking between the modular automation drive unit 102 and the TTT device 200 can be achieved via magnetic attraction. For example, a first magnet is embedded within the housing (e.g., in place of the hole 107), and a second magnet is embedded in place of the female threaded hole 109. The two magnets are oriented such that when brought together, their opposite poles face each other (e.g., North to South) and are thus configured to magnetically engage each other. The coupling between the modular automation drive unit 102 and the TTT device 200 can additionally or alternatively be achieved by magnetic coupling between the two devices wherein the male threaded tip 116 is embedded with one or multiple magnets with single or multiples poles, or is magnetized itself in this manner, while the non-circular cavity 295 can be surrounded by or embedded with one or more magnets with opposite polarities facing the magnet(s) in the (magnetized) male threaded tip 116. Again, North to South, whether one pair or multiple pairs of magnets.

The microcontroller 129 is configured to output a control signal that is delivered to the motor 128 via the connector 130 and wires 131. The control signal is configured to direct the motor to rotate the driven output hex 116 in a first rotational direction and/or a second, opposite rotational direction, at a constant rotational speed, or at more than one rotational speeds, at accelerating speeds or decelerating speeds, or at a pattern of different speeds and/or different changes in speed or velocity. In some embodiments, the microcontroller 129 is programmed and/or programmable to automatically cause rotation of the driven output hex 116 at a rotational velocity that that changes over time. In some embodiments, the microcontroller 129 is programmed and/or programmable to automatically cause rotation of the driven output hex 116 at a rotational velocity that that changes over time according to a non-linear function. The non-linear function can comprise one or more of: an exponential function, a logarithmic function, a polynomial function, a quadratic function, a growth function, a delay function, a step function, and/or a decay function.

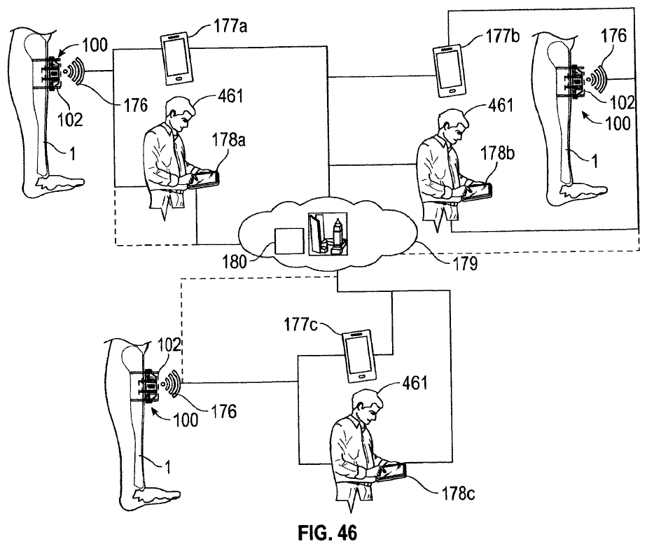

In some embodiments, the circuitry 127 is coupled to one or more sensor 164 (FIG. 22) configured to measure a parameter within the patient 16 and to output a signal indicative to the measured parameter. In some embodiments, the circuitry 127 can be directly coupled to the sensor 164 via a wired connection. In other embodiments, the circuitry 127 includes a receiver 165 (FIG. 20) configured to receive a wireless signal 176 (FIG. 46) output by the sensor 164 and indicative of the measured parameter. The wireless communication can include, but is not limited to: Bluetooth, BLE (Bluetooth low energy), NFC (near field communication), Wifi, or 4G/5G. In some embodiments, a smart device 177, 178 such as a smartphone 177a-c or a smart tablet 178a-c, or an app 180 on a cloud computing system 179 can be configured to receive a wireless signal 176 output by the sensor 164 and indicative of the measured parameter. The sensor 164 in some embodiments is a force transducer configured to measure force on tissue of the patient 16, including a force on a bone of the subject or a bone piece (for example, the tibia 1 and the bone piece 216). The sensor 164 in some embodiments is a temperature sensor (thermocouple, thermistor, RTD) configured to measure a temperature of tissue of the patient 16, including tissue adjacent a bone of the subject or a bone piece (for example, the tibia 1 and the bone piece 216). In some embodiments, the sensor 164 comprises a light emitter and light detector, and can be configured to utilize spectroscopy, e.g., for measuring oxygenation in the tissue. In some embodiments, the spectroscopy comprises near-infrared spectroscopy. The tissue can comprise soft tissue, such as muscle, fat, tendon, ligament, nervous tissue, and other connective tissue. Certain changes in temperature, such as sudden increases in temperature, can indicate fever, injury, inflammation, infection, or irritation. The sensor 164 in some embodiments is an accelerometer configured to measure movement of the patient 16. The sensor 164 in some embodiments is an Inertial Measurement Unit (IMU) sensor configured to measure, track, and analyze body movement of the patient 16, such as acceleration, orientation, angular change rates, and gravitational forces. The IMU can comprise one or more accelerometers, one or more gyroscopes, and one or more magnetometers. In some cases, the movement can be movement of the patient 16 or of a limb or appendage of the patient 16. In some cases, the movement can be movement of the bone piece 216.

In other embodiments, the microcontroller 129 is instead a control unit that is carried by a cloud computing system 179 and is configured for wireless two-way communication, either with one or more modular drive units 102, directly or via a smart device App 180. The cloud computing system 179 is configured to be linked to a website or user interface platform where users can wirelessly view all of the data information and status related to the system 100. The data information and status are either stored in the cloud computing system 179, itself, or is stored within the modular drive unit 102, e.g., in a memory device, and which is then transmitted to the cloud computing system 179. The information can also be input into the system securely through the cloud 179 by a main user 461 or other personnel, or the patient and family members. The information can include one or more prescriptions, or other command information, such as patient action information or device command information. In other embodiments, the control unit comprises a portable smart device App 180 and is configured for wireless two-way communication with the modular drive unit 102. The portable smart device App 180 is configured for multiple platforms such as smart phones 177 or tablets 178 on different operating systems such as Android or iOS. The App 180 is configured to be securely two-way communicating with the system wirelessly, including receiving information from one or more sensors, and receiving system status information, as well as sending commanding and driving signals to the system, and instructions to the patient, either for their personal actions or in their actions operating the modular drive unit 102. The App 180 is also configured to display the system information either graphically or through audible sound, or through videos. The App 180 is also configured for users to input the prescription into the App 180 interactively. The App 180 is configured to be able to wirelessly two-way communicate with other multiple systems. The App 180 is also configured to be able to securely two way communicate with the cloud computing system 179. The display can be presented on an external device, the modular drive unit 102, itself, or on a website that can be viewed by a number of devices, including computing devices or monitors.

The modular drive unit 102 can be configured to directly two-way communicate with smart devices 177, 178, or to communicate directly with the central cloud 179. Multiple modular drive units 102 can directly two-way communicate with the central cloud 179. The App 180 can be loaded onto the smart phone/mobile phone 177 or tablet 178 or a personal computer (PC). Administrators, surgeons, hospital staff, sales representatives, or clinical specialists, or even patients and their friends and family (e.g., users 461) can view the progress of one or more systems 100 through the central cloud 179 as allowed per their specific viewing rights and permissions. The user 461 can change the prescription of one or more systems 100, through the central cloud 179, either directly or through the smart device 177, 178. The access to the App 180 and to the Cloud 179 can be password protected, and can include multiple authentication methods.

The microcontroller 129 can comprise a processor configured to receive the signal from the sensor 164 (or a plurality of sensors) directly or a processed or conditioned version of the signal from the sensor 164 and to compare the signal received with data and/or instructions stored on a non-transitory computer-readable medium which when executed by the processor configures the processing unit to execute a routine for operating the motor 128 and rotating the driven output hex 116. In some embodiments the medium comprises a memory 169 carried by the circuitry 127. In some embodiments, the microcontroller 129 comprises a processor that is configured to modify the instructions stored on a non-transitory computer-readable medium. In some embodiments, the microcontroller 129 comprises a processor that is configured to modify the instructions stored on a non-transitory computer-readable medium based at least in part on the signal from the sensor 164, for example the signal received by the receiver 165. Modifying the instructions can comprise changing from one preset routine for operating the motor to another preset routine for operating the motor 128. Modifying the instructions can comprise changing values in a preset routine to create a new routine. The values being changed can comprise, voltage applied, current generated, rise time, fall-off time, slew rate, hold duration, and time at completion. In some embodiments, the microcontroller 129 comprising a microprocessor can be configured to measure current that is run through motor, real-time, or with a delay, and to analyze the current. If sudden changes or changes above a threshold are measured, the microprocessor is configured to adjust the motor speed by applying a different voltage. This technique can mitigate excessive tissue tension or stress, to optimize clinical outcome.

In some embodiments, the circuitry 127 includes an artificial intelligence (AI) system 166 (FIG. 20) configured to be embedded with the processor or to integrate with the processor. The processor can comprise the microcontroller 129, or can comprise a separate microprocessor carried by the circuitry 127 or configured to communicate with the circuitry 127. In some embodiments, a smart device or an app on a cloud computing system can be configured to receive information output by AI system 166. In some embodiments, the microcontroller 129 is configured to respond to information output by the AI system 166 to change from one preset routine to another preset routine, or to modify values of a preset routine. In some embodiments, the AI system 166 is configured to provide an optimized prescription for the patient 16. The optimized prescription can comprise one or more portions of a routine, or a changed routine for operating the motor 128 and the driven output hex 116. For example, the AI system 166 can compare the initial data from the patient 16, in terms of any one or more values received from one or more sensors 164. The AI system, 166 can then compare these data with historical data from one or more database, and determine whether and how to modify parameters to optimize a prescription at that time for the patient 16. The optimized prescription can include instructions stored on a non-transitory computer-readable medium to automatically cause rotation of the driven output hex 116 at a rotational velocity that that changes over time according to a non-linear function. The non-linear function can comprise one or more of: an exponential function, a logarithmic function, a polynomial function, a quadratic function, a growth function, a delay function, a step function, and/or a decay function.

In another alternative embodiment, a physical energy generator 168 is carried on or in the modular drive unit 102 and is configured to generate a visible, audible, or tactile alert, or alarm, that is perceptible by the user. Thus, physical energy generator can be electrically coupled to the circuitry 127 and can be configured to be initiated by any signal received from a sensor 164, and/or from information received from the AI system 166, and/or from control instructions received from a memory 169 or from a smart device/mobile device, and/or from an application on a cloud computing system. The physical energy generator 168 can comprise a light, an LED, a flashing light, a flashing LED, a loudspeaker, a piezoelectric configured to vibrate, or a mechanical vibrator or mechanical noisemaker such as a clicking device or a buzzer. In another embodiment, the physical energy generator 168 is configured to generate a visible, audible, or tactile alert, perceptible by the user, that indicates when the modular drive unit 102 has been correctly coupled to the TTT device 200. For example, this can constitute all of alignment steps except the locking securement using the locking screw 101. Or, it can constitute all of the alignment steps and the locking securement using the locking screw 101. In some embodiments, the modular drive unit 102 and the TTT device 200 comprise Hall effect sensors or other proximity sensors that provide a signal to the circuitry 127 when the modular drive unit 102 and the TTT device 200 are separated by a distance less than a threshold distance. Thus, a processor reacts to a signal that is greater than a minimum or threshold signal emanating from the Hall effect sensor. In some embodiments, the threshold cannot be reached unless the alignment steps have all been performed, because otherwise the lower ledge face 113 would be able to sufficiently come close to the raised access area 252. In another embodiment, one or multiple Hall effect sensors are carried in or on the modular drive unit 102, and can detect the rotation of the leadscrew 274, or just a portion of the leadscrew 274, such as the head 281, or the non-circular cavity 295. For example, any portion of the leadscrew 274 comprises a magnet with single or multiple poles whose locations change as the leadscrew 274 is rotated. Thus, the magnetic fields of the one or more magnets move in concert with the rotation of the leadscrew 274. The control unit can receive and process signals from the one or more Hall effect sensors, to confirm the rotation of the leadscrew 274, and thus confirm a stable coupling between the modular automation drive unit 102 and the TTT device 200 or PD device 300. In another embodiment, one or more proximity sensors are carried in or on the modular drive unit 102, and can detect the distance to or proximity of the movable stage 219, 319 inside the TTT device 200 or PD device 300, wherein movable stage 219, 319 comprises a magnet with single or multiple poles. Thus, the proximity sensor(s) can confirm the movement of the movable stage 219, 319, and thus confirm stable coupling between the modular automation drive unit 102 and the TTT device 200 or PD device 300.

FIG. 80 illustrates a PD device 1157 comprising a distraction plate 1158 configured for implantation under a periosteum 1159, and an implantable actuator 1160 comprising a base 1161, and a distraction piston 1162. The upper end 1163 of the piston 1162 is attached to the plate 1158, at a central portion 1164. A first coil 1165 is carried within the base 1161, adjacent the piston 1162 and is coupled to the piston 1162. The coil 1165 comprises shape-memory alloy wire (e.g., nitinol) and is configured to lengthen when heated (e.g., approaching or above an austenitic (final) (A_{F}) temperature). The transverse expansion of the coil causes the piston 1162 to extend transversely, and to push the plate 1158 away from the bone, stretching the periosteum 1159. In some embodiments, the end 1163 of the piston 1162 is not attached to the plate 1158, but only abuts it. An external device 1166 comprises a driving coil 1167 (e.g., copper wire coil) configured to induce current within the coil 1165, so that the coil 1165 heats up from its inherent electrical resistance. The external device 1166 further comprises a user interface 1167 to allow a user to operate it, assess it, or program it.

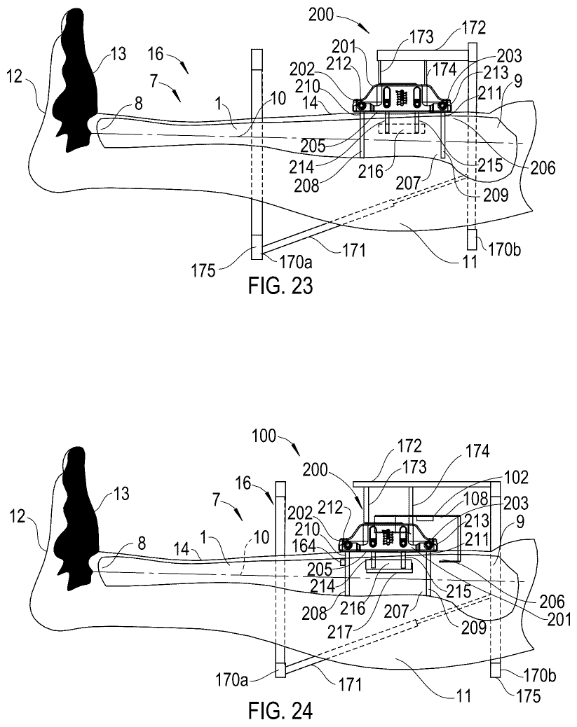

FIGS. 23 and 24 illustrate, respectively, a system 100 for increasing biological activity within a patient comprising the transverse tibia transport device (TTT device) 200 and a modular drive unit 102, coupled to an external fixator 175, and a transverse tibia transport device (TTT device) 200, coupled to an external fixator 175. Many patients 16 may have other orthopedic anomalies or morbidities, as well as being treated with transport device (TTT device) 200. In some patients, the orthopedic anomalies or morbidities may be been exacerbated by a diabetic condition of the patient. In some patients, the orthopedic anomalies or morbidities may have been at least partially caused or initiated by a diabetic condition of the patient. It may be desired to treat the orthopedic anomalies or morbidities while also performing therapy to increase biological activity with the transverse tibia transport device (TTT device) 200 alone or with the transverse tibia transport device (TTT device) 200 in combination with the modular drive unit 102.

The external fixator 175 comprises one or more rings 170, and is generically represented by a distal ring 170a and a proximal ring 170b. The rings 170 each extend at least partially around the lower leg 7 of the patient 16. One or more connecting rods 171 are coupled to the rings 170 and are sized and adjusted accordingly for the patient's needs. A longitudinal bar 172 is coupled to the proximal ring 170b, and extends distally. The longitudinal bar 172 is sufficiently stiff and has a sufficient diameter to remain rigid without significant bending. In other embodiments, the longitudinal bar 172 can be coupled to the distal ring 170a, and/or can extend proximally. Two fixation rods 173, 174 attach to the longitudinal bar 172 at first ends, and attach to the TTT device 200 at second ends. Thus, the TTT device 200 is further held in place and maintained in a stationary condition, by the attachment to the external fixator 175.

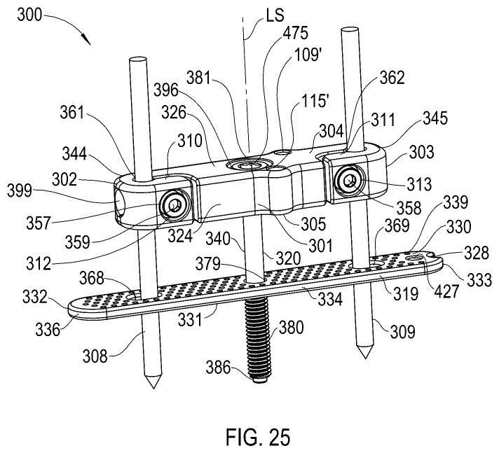

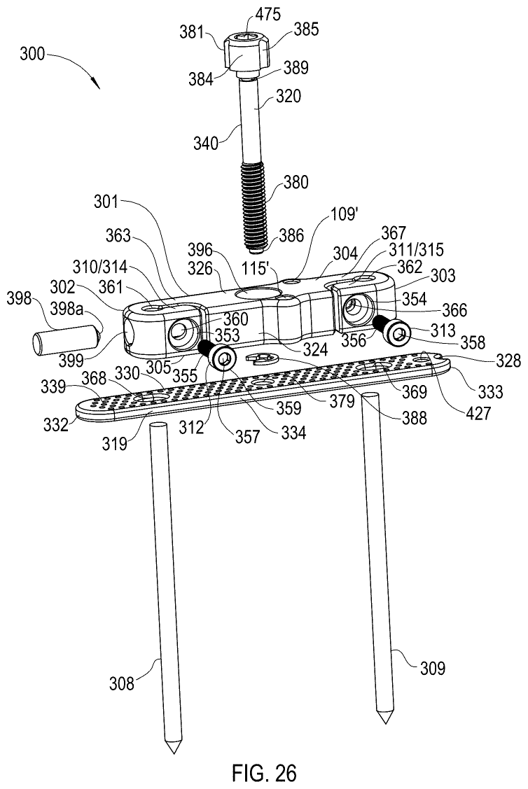

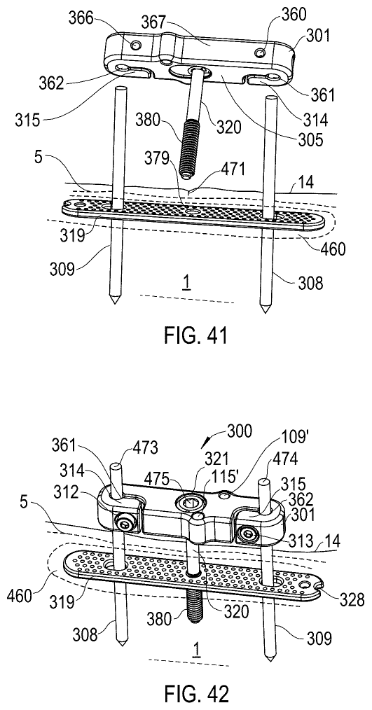

Returning to the periosteal distraction device (PD device) 300 of FIGS. 5-6, further details are illustrated in FIGS. 25-26, 31-32, and 41-42. The base 301 comprises a first end 302, and second end 303, an upper surface 304, and a lower surface 305. The upper surface 304 and the lower surface 305 each extend between the first end 302 and the second end 303. The base 301 includes a central portion 324 having a substantially planar top area 326 of the upper surface 304. The central portion 324 includes a hole 396 in which a head 381 of a leadscrew 320 is carried. The leadscrew 320 is rotatably locked underneath the lower surface 305 of the base 301 by a C-clip 388 (snap ring) (FIGS. 26 and 41), that is snapped around a circumferential groove 389 in the leadscrew 320, beneath the head 381. This substantially maintains the longitudinal position of the leadscrew 320 relative to the base 301, which still allowing the leadscrew 320 to rotate about a leadscrew axis LS.

A movable stage 319, comprising a plate, is configured to engage an internal, underneath portion of periosteum 5 of the bone (e.g., tibia 1). The movable stage 319 comprises an upper surface 330, a lower surface 331, a first end 332, a second end 333, a front edge 334, and a back edge 335. The first end 332 comprises a semi-circular curve 336, and the second end 333 comprises a partial semi-circular curve 337, having a smaller semi-circular notch 328. The movable stage 319 includes a series of transverse holes 339. The movable stage 319 further comprises an internal thread 379 configured to threadingly engage an external thread 380 of the leadscrew 320. The leadscrew 320 further comprises a shaft 340 extending distally from the head 381. The leadscrew 320 also comprises a distal cylindrical projection 386. The movable stage 319 additionally has an internal thread 427 passing therethrough and adjacent the semi-circular notch 328, which will be described in detail later.

Bone anchors 308, 309, are statically coupled to the base 301 by compressing clamping portions 310, 311 of the base 301 with set screws 312, 313, respectively. The movable stage 319 includes two holes 368, 369 that are larger than the diameter of the bone anchors 308, 309 and are configured to pass over the bone anchors 308, 309, respectively, to allow the movable stage 319 to translate over the bone anchors 308, 309, being guided, but not impeded by them. The first clamping portion 310 of the base 301 comprises a first counterbore hole 353 configured to rotatably capture a head 359 of the first set screw 312. An internal thread 360 (FIG. 41) in a rear section 363 threadingly engages a male thread 355 of the first set screw 312. The second clamping portion 311 of the base 301 comprises a second counterbore hole 354 configured to rotatably capture a head 364 of the second set screw 313. An internal thread 366 (FIG. 41) in a rear section 367 threadingly engages a male thread 356 of the second set screw 313. The set screws 312, 313 each comprise a screw drive 357, 358 for keyingly engaging a drive tip of a torque tool, such as a screwdriver or a motorized driver. The screw drives 357, 358 can comprise any non-circular cavity shape, but can alternatively comprise a non-circular protrusion/protuberance. The non-circular shape of the screw drives 357, 358 can comprise any one of a hex, a torx, a slot, a Phillips or other X-shape or cruciform shape, a Robertson or other square shape, a security or tamper-resistant shape, an oval, a spanner, a pentalobular, a tri-point, a multiple square, or any combination thereof.

A first end portion 344 includes a vertically-extending hole 361 passing therethrough, and a second end portion 345 includes a vertically-extending hole 362 passing therethrough. The holes 361, 362 are sized to allow the bone anchors 308, 309 to be passed therethrough. By tightening the set screws 312, 313, the heads 359, 364 are pulled toward the rear sections 363, 367, thus flexing the beam portions 314, 315 inwardly. This flexure causes the effective inner diameter of the holes 361, 362 to decrease, such that the inner surfaces around the holes 361, 362 grips the bone anchors 308, 309 firmly on their shafts.

Prior to engaging the thread 355 of the set screw 312 during assembly of the PD device 300, a ball spring plunger 398 having a spring-loaded ball 398a (similar to ball spring plunger 298) is slid into transverse hole 399 (FIG. 26) which continues to the center of the base 301, communicating with the hole 396. The ball spring plunger 398 can be press-fit into the hole 396 such that the ball 398a has a similar relationship with the head 381 of the leadscrew 320, as does the ball 298a with the head 281 of the leadscrew 274. The three convex arc portions 384 are equally distributed around the leadscrew axis LS, and three concave depressions 385 are evenly distributed between the convex arc portions 384.

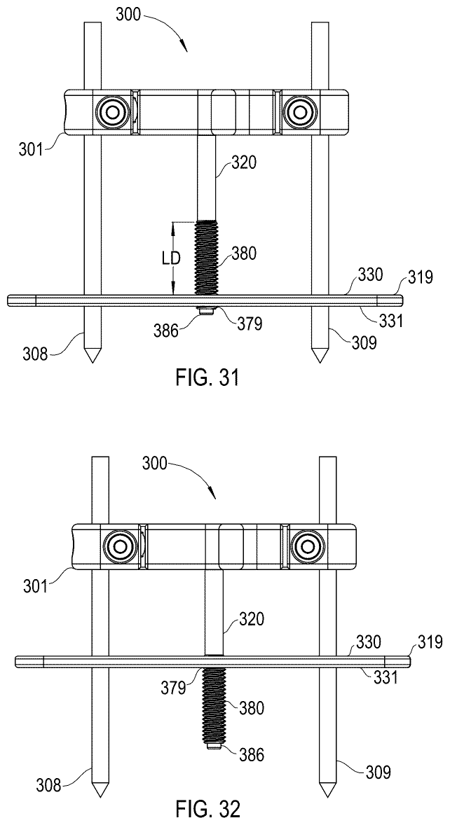

A physical reaction occurs between a concave depression 385 and the ball 398a of the ball spring plunger 398 when the leadscrew 320 is moved from a non-engaged position (ball 398a compressed by convex arc portion 384) to an engaged position (ball 398a allowed to release into concave depression 385). This physical reaction (e.g., of a first metal piece accelerating into and striking another metal piece) produces a physical disturbance configured to be sensed by the user as an audible sound and/or as a tactile vibration. This disturbance provides information to the user, via the user's ears and/or fingers/hands/arm or other body part or sense, and it relates to the user the extent of relative displacement between the translatable anchor and the base. The physical disturbance can be configured, as previously described in relation to the ball spring plunger 298 and the leadscrew 274. Thus, the amount of movement of the movable stage 319, and thus the amount of stretching of the periosteum 5 can be quantified in real-time by the user. FIG. 31 illustrates the PD device 300 in a first distraction position, with the movable stage 319 at the bottommost extent in relation to the external thread 380 of the leadscrew 320. In procedures in which the periosteum 5 is to be distracted away from its related bone, the position of FIG. 31 would often be the starting position. However, in some cases, if the expected amount of total distraction expected in the patient is less than the total displacement length LD, it may be desired to begin with the movable stage 319 in a higher, pre-displaced position than that shown in FIG. 31. FIG. 32 illustrates the PD device 300 in a second distraction position, with the movable stage 319 at the topmost extent in relation to the external thread 380 of the leadscrew 320. The external thread 380 can in some embodiments include lead in portions at each end, to avoid any possible locking up between the external thread 380 and the internal thread 379. The external thread 380 can in some embodiments be fabricated to extend beyond the possible translation of the movable stage 319 at each end, each direction of travel, to avoid bottoming out.

Like the TTT device 200, the PD device 300 can be operated manually, by use of a torque tool, or can be coupled with the modular drive unit 102 for automatic operation. All of the capabilities of the system 100 are possible with a system coupling the PD device 300 with the modular drive unit 102, including wired or wireless communication, control via a microcontroller in the drive unit 102, or by a smart device, or from a cloud computing system. All of the types of control, and control algorithms, are also possible, as well as the incorporation of artificial intelligence (AI).

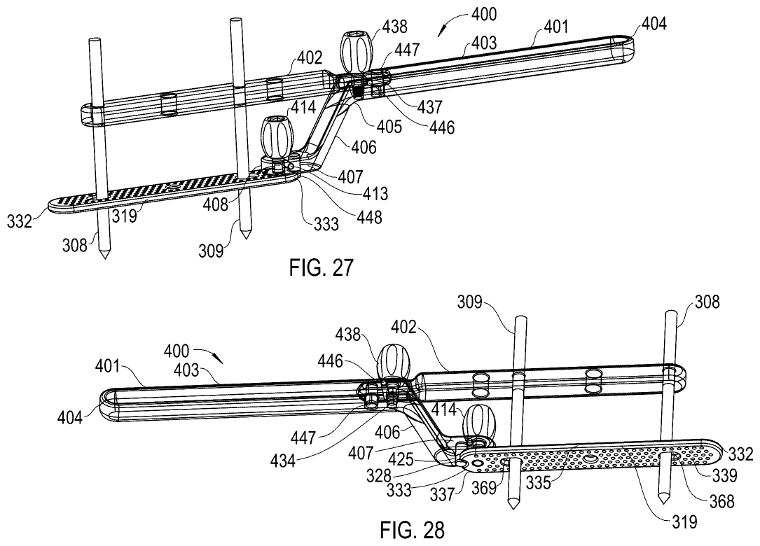

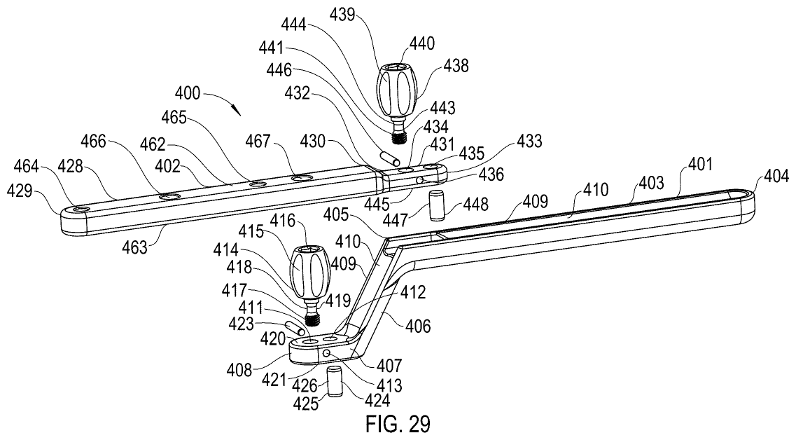

Turning to FIGS. 27-30, an insertion tool 400 is configured for inserting the movable stage of the PD device 300 and the bone anchors 308, 309 within a patient and enabling correct attachment of the PD device 300 to the patient. The movable stage 319 is to be inserted through an incision 454 in the skin (FIG. 36) and an incision 457 in the periosteum 5 (FIG. 35). The base 301 of the PD device 300 is to be carried on or above the skin 14 of the patient 16. Thus, the insertion tool 400 also serves as an alignment tool, to couple the bone anchors 308, 309 with the movable stage 319. FIG. 29 illustrates the components of the insertion tool 400. The insertion tool 400 comprises two main elongate bodies/arms, a stage-holding arm 401 and an attachable/detachable targeting arm 402. Each of these arms 401, 402 comprises a low-density, biocompatible metal, such as anodized aluminum, or alternatively a high-strength engineering plastic or polymer composite.

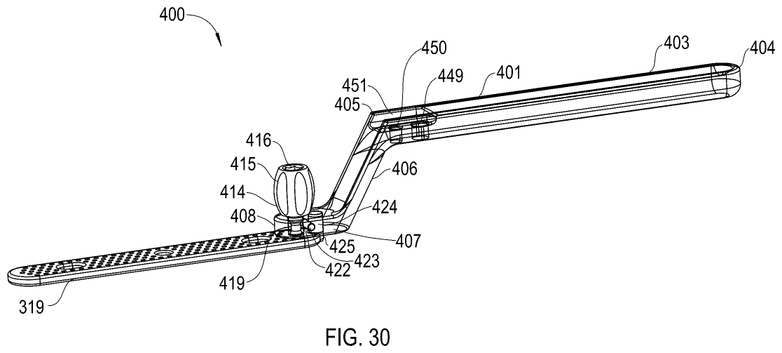

The stage-holding arm 401 comprises an elongate proximal handle 403 having a proximal end 404 and a distal end 405, an angled transition portion 406, and a distal connection portion 407. The angled transition portion 406 extends between the distal connection portion 407 and the distal end 405 of the handle 403. The proximal end 404 of the handle 403 and the distal end 408 of the distal connection portion 407 represent the proximal end and the distal end of the stage-holding arm 401, respectively. The handle 403 and the transition portion 406 comprise rail portions 409 on the lateral edges for maintaining relatively high bending stiffness, and a hollowed-out central portion 410 for reduced overall weight of the arm 401. The distal connection portion 407 includes a vertically-extending tightening nut clearance hole 411, a vertically-extending dowel pin hole 412 that is substantially parallel to the clearance hole 411, and a horizontally-extending dowel pin hole 413. The longitudinal distance between centers of the nut clearance hole 411 and the horizontally-extending dowel pin hole 413 is configured such that there is communication 422 (FIG. 30) between them. In other words, this distance is less than the sum of the radius of the nut clearance hole 411 plus the radius of the horizontally-extending dowel pin hole 413. The communication 422 comprises a longitudinally-extending opening between the nut clearance hole 411 and the horizontally-extending dowel pin hole 413. A tightening screw 414 includes a proximal knurled handle 415 having a proximal screw drive 416, a distal male threaded portion 417, and a central shaft 418 extending between the handle 415 and the male threaded portion 417. The shaft 418 comprises an hourglass contour 419 comprising a circumferential concavity having a radius. The male threaded portion 417 is inserted through the clearance hole 411 from a top face 420 of the distal connection portion 407 such that it extends out from a bottom face 421 of the distal connection portion 407. In this position, the hourglass contour 419 of the shaft 418 resides within the clearance hole 411, immediately adjacent to the communication 422 between the clearance hole 411 and the dowel pin hole 413. A dowel pin 423 is press fit into the dowel pin hole 413, which locks the tightening screw 414 in place via the hourglass contour 419, while still allowing free rotation of the tightening screw 414.

An indexing dowel pin 424 is press fit into the vertically-extending dowel pin hole 412. Thus, a lower portion 425 of the dowel pin 424 extends below the bottom face 421 of the distal connection portion 407 for indexing with the movable stage 319, and the distal male threaded portion 417 of the tightening screw 414 is rotatable below the bottom face 421 of the distal connection portion 407 via manipulation of the knurled handle 415 or the screw drive 416 (e.g., via a manual or motorized torque tool). The semi-circular notch 328 in the movable stage 319 is shown in FIGS. 25, 26, and 28. In use, a user indexes the stage-holding arm 401 with the movable stage 319 by placing a distal portion of the outer diameter 426 flush against the semi-circular notch 328. This stabilizes the longitudinal relationship between the stage-holding arm 401 and the movable stage 319 as well as the lateral relationship of the second end 333 of the movable stage with respect to the stage-holding arm 401. The user then places the distal male threaded portion 417 of the tightening screw 414 adjacent the matching internal thread 427 in the movable stage 319, and tightens the threaded portion 417 into the internal thread 427 to lock the stage-holding arm 401 to the movable stage 319 in their intended relative positions.

The targeting arm 402 is then coupled to the stage-holding arm 401. The targeting arm 402 is configured to deliver the bone anchors 308, 309 through the skin 14 of the patient 16 and through the holes 368, 369 of the movable stage 319. The targeting arm 402 comprises an elongate targeting portion 428 having a distal end 429 and a proximal end 430, and a handle-connection portion 431 having a distal end 432 and a proximal end 433. The proximal end 433 of the handle-connection portion 431 and the distal end 429 of the targeting portion 428 define the proximal end and the distal end, respectively, of the targeting arm 402. The handle connection portion 431 includes a vertically-extending tightening nut clearance hole 434, a vertically-extending dowel pin hole 435 that is substantially parallel to the clearance hole 434, and a horizontally-extending dowel pin hole 436. The longitudinal distance between centers of the nut clearance hole 434 and the horizontally-extending dowel pin hole 436 is configured such that there is communication 437 (FIG. 27) between them. In other words, this distance is less than the sum of the radius of the nut clearance hole 434 plus the radius of the horizontally-extending dowel pin hole 436. The communication 437 comprises a longitudinally-extending opening between the nut clearance hole 434 and the horizontally-extending dowel pin hole 436. A tightening screw 438 includes a proximal knurled handle 439 having a proximal screw drive 440, a distal male threaded portion 441, and a central shaft 442 extending between the handle 439 and the male threaded portion 441. The shaft 442 comprises an hourglass contour 443 comprising a circumferential concavity having a radius. The male threaded portion 441 is inserted through the clearance hole 434 from a top face 444 of the handle-connection portion 431 such that it extends out from a bottom face 445 of the handle-connection portion 431. In this position, the hourglass contour 443 of the shaft 442 resides within the clearance hole 434, immediately adjacent to the communication 437 between the clearance hole 434 and the dowel pin hole 436. A dowel pin 446 is press fit into the dowel pin hole 436, which locks the tightening screw 438 in place via the hourglass contour 443, while still allowing free rotation of the tightening screw 438.

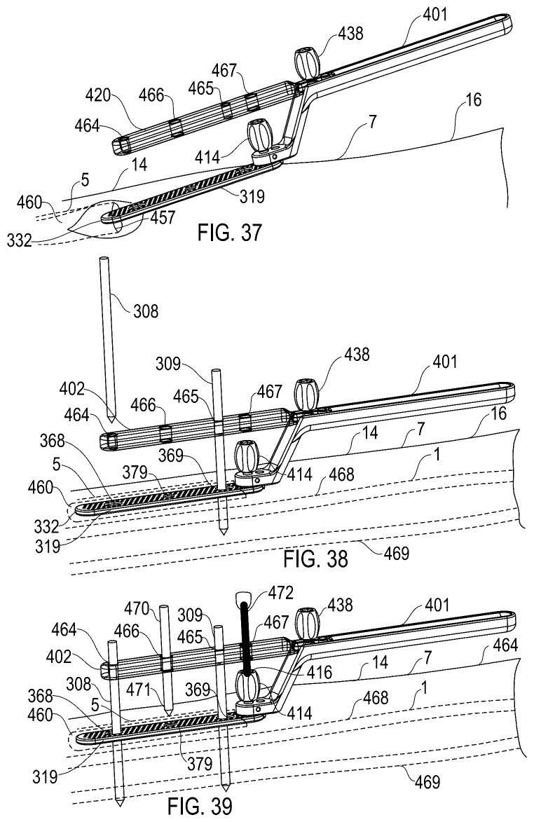

An indexing dowel pin 447 is press-fit into the vertically-extending dowel pin hole 435. Thus, a lower portion 448 of the dowel pin 447 extends below the bottom face 445 of the handle-connection portion 431 for indexing with the stage-holding arm 401, and the distal male threaded portion 441 of the tightening screw 438 is rotatable below the bottom face 445 of the handle-connection portion 431 via manipulation of the knurled handle 439 or the screw drive 440 (e.g., via a manual or motorized torque tool). FIG. 30 shows the stage-holding arm 401 coupled to the movable stage 319 without the targeting arm 402 attached. This allows better visibility of the features of the stage-holding arm 401. Although the insertion and attachment steps shown in FIGS. 37-39 utilize both the stage-holding arm 401 and the targeting arm 402 attached together, in alternative insertion methods, the movable stage 319 can be inserted with the use of the stage-holding arm 401, without the targeting arm 402. The handle 403 of the stage-holding arm 401, adjacent its distal end 405 comprises a vertically-extending indexing hole 449 and a vertically-extending female threaded hole 450. Both of these holes 449, 450 are within a recessed portion 451. The recessed portion 451 is configured to fit the handle-connecting portion 431 of the targeting arm 402. In use, a user orients and indexes the targeting arm 402 with the stage-holding arm 401 by placing the handle-connecting portion 431 of the targeting arm 402 flush within the recessed portion 451. This stabilizes the longitudinal and lateral relationship between the targeting arm 402 and the stage-holding arm 401. The user then places the lower portion 448 of the dowel pin 447 into the indexing hole 449. The user then places the distal male threaded portion 441 of the tightening screw 438 adjacent the matching internal thread 450 in the stage-holding arm 401, and tightens the threaded portion 441 into the internal thread 450 to lock the targeting arm 402 to the stage-holding arm 401 in their intended relative positions. Turning to FIG. 29, the targeting arm 402 comprises an upper planar face 462 and a lower face 463. The upper planar face 462 is on the targeting portion 428, and provides first and second bone anchor targeting holes 464, 465, a leadscrew access hole 466, and an access hole 467, for accessing the proximal screw drive 416 of the tightening screw 414. Each of these holes 464, 465, 466, 467 passes completely through the targeting portion 428 in a substantially vertical direction. In alternative embodiments, one or more of the holes 464, 465, 466, 467 can comprise a counterbore and/or a tapered diameter. For example, a proximal (upper) portion of the hole can have a tapered lead-in for facilitated insertion of a bone anchor 308, 309 or a tightening/torquing tool. However, the holes 464, 465, 466, 467 typically have a close-fitting section having sufficient length and sufficient length-to-diameter ratio to accurately aim the bone anchor 308, 309 in the desired direction.

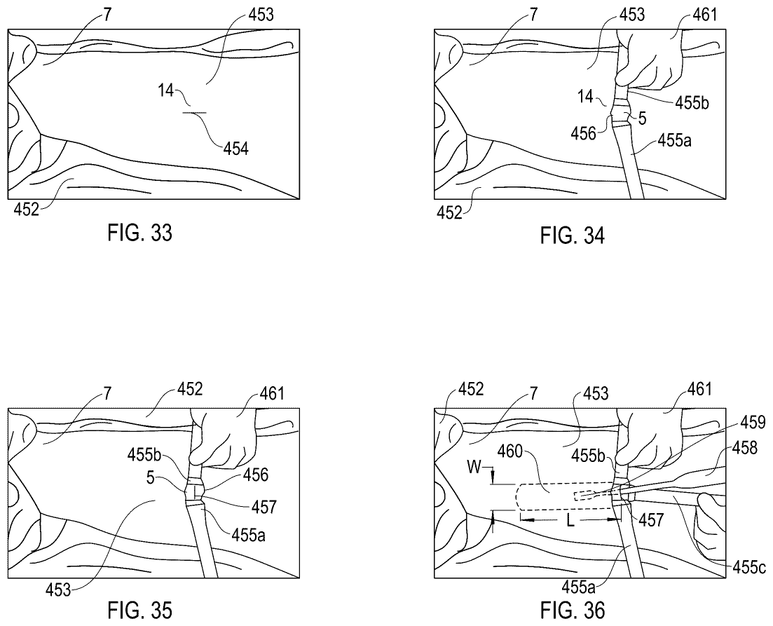

As shown in FIG. 37, the movable stage 319 is now ready for placement beneath the periosteum 5 of a patient 16. The steps of preparing a patient for implantation of the movable stage are shown in FIGS. 33-36. Surgical drapes 452 are placed around the operating area 453 of a lower leg 7 of a patient. Anesthesia is initiated in the patient 16. The anesthesia can be local or epidural, and in some cases can be regional or general. In FIG. 33, a longitudinal incision 454 is made in the skin 14 of the patient 16 in the operating area 453, by use of a scalpel, or other cutting instrument. The incision 454 extends through subcutaneous fat and connective tissue between the skin 14 and the periosteum (e.g., tibia 1, as shown, or other bone). A typical length for the incision 454 can be approximately 20 mm. In FIG. 34, a user 461 utilizes surgical retractors 455a, 455b to retract the skin 14 to change longitudinal incision 454 to an opening 456, exposing the periosteum 5 that surrounds the tibia 1. In FIG. 35, a transverse incision 457 is made in the periosteum 5, by use of a scalpel, or other cutting instrument. A typical length for the incision 457 can be approximately 10 mm. An appropriate width is chosen in order to access the subsequent operations, as described. In FIG. 36, a periosteal elevator 458 having a separator tip 459 is used to separate the periosteum 5 from the tibia 1, and to create a longitudinal, subperiosteal tunnel 460, for placement of the movable stage 319. One or more additional retractors 455c can be used by the user 461 or other personnel, if additional retraction is required. For example, the retraction can be in a longitudinal direction, such as an opposite direction of the advancement of the tip 459 of the periosteal elevator 458. The tunnel 460 has a width W and a length L that are large enough to allow insertion of the movable stage 319. The tunnel 460 can be made to be initially very small in terms of space away from the tibia 1, if substantial dissection is avoided. This allows for maximal stretchability of the periosteum 5. For example, when the incision 457 is made, the space can be less than the thickness of the movable stage 319 (e.g., distance between the upper surface 330 and the lower surface 331). Careful placement of the movable stage 319 into the tunnel 460 can cause minimal initial stretching. The width W and length L do not necessarily need to be wider and longer than the movable stage 319, for example, if the periosteum is able to stretch somewhat during insertion of the movable stage 319. In some embodiments, the movable stage 319 can comprise a plate (e.g., “lift plate”) having an 8 mm width and 80 mm length, and a low thickness (e.g., less than 5 mm, or less than 4 mm, or less than 3 mm, or less than 2 mm). Holes can be drilled in the near cortex of the tibia 1 at the site of the incisions 454, 457, to decompress the medullary cavity. In some cases, there may be four holes drilled with a 2 mm diameter drill bit. The holes can be placed at four corners of a square area. For example, each hole can be approximately 10 mm from the center of the incision 454 in both a positive or negative X-direction, and a positive or negative Y-direction. The tunnel 460 is shown in one embodiment in FIG. 36, wherein it substantially extends distally to the incisions 454, 457. However, the periosteal elevator 458 can be used bi-directionally (distal and proximal to the incisions 454, 457) such that the longitudinal center point of the tunnel 460 is at the incisions 454, 457. Thus, an additional center hole would not have to be made in the skin 14, because the center of the movable stage 319 would be accessible through the incisions 454, 457.

As shown in FIG. 37, a user holds the handle 403 of the stage-holding arm 401, and inserts the first end 332 of the movable stage 319 into the transverse incision 457 in the periosteum 5. The user then advances the movable stage 319 into the longitudinal tunnel 460 until the movable stage 319 is substantially within the tunnel 460, as shown in FIG. 38. The center of the movable stage 319, for example can be moved forward or backward, to align the internal thread 379 approximately with a center or middle of the incision 454. The bone anchor 309 is first passed through the proximal bone anchor hole 465 of the targeting portion 428 of the targeting arm 402, then either piercing the skin, or passing through a pre-made incision in the skin, then passed through the hole 369 in the movable stage 319, and then hammered or screwed into the near cortex 468 of the tibia 1, as shown in FIG. 38. FIG. 38 also shows a second bone anchor 308 about to be inserted through the distal bone anchor hole 464. FIG. 39 shows both bone anchors 308, 309 after they have been passed through the near cortex 468, and also the far cortex 469 of the tibia 1. In some cases, a user may decide to only secure one or both of the bone anchors 308, 309 only to the near cortex 468 and not the far cortex 469. FIG. 39 illustrates the distal attachment into the far cortex 469, using both bone anchors 308, 309. The distal bone anchor 308 is first passed through the proximal bone anchor hole 464 of the targeting portion 428 of the targeting arm 402, then either piercing the skin, or passing through a pre-made incision in the skin, then passed through the hole 368 in the movable stage 319, and then hammered or screwed into the near cortex 468 of the tibia 1, and/or the far cortex 469. The holes 464, 465 serve to accurately aim and target the holes 368, 369 in the movable stage 319. In alternative embodiments, a single bone anchor can be sufficient for securely and rigidly anchoring the base 301. In a subsequent step, the base 301 of the PD device 300 will be attached, and the leadscrew 320 will also be passed through the skin 14. FIG. 39 illustrates a puncturing tool 470 being passed through the leadscrew access hole 466 of the targeting portion 428, and making a puncture 471 in the skin 14 of the patient 16. The puncturing tool 470 is intended to make space for the leadscrew 420 in the correct direction, and thus creates a channel through the skin for the leadscrew 420 to pass toward the internal thread 379 in the movable stage 319, into which the external thread 380 of the leadscrew 320 can be threadingly engaged. After creating the puncture 471, the puncturing tool 470 can be removed. In some cases, an additional puncture in the skin 14 would not need to be made for the bone anchor 309, as it could extend through the incision 457.

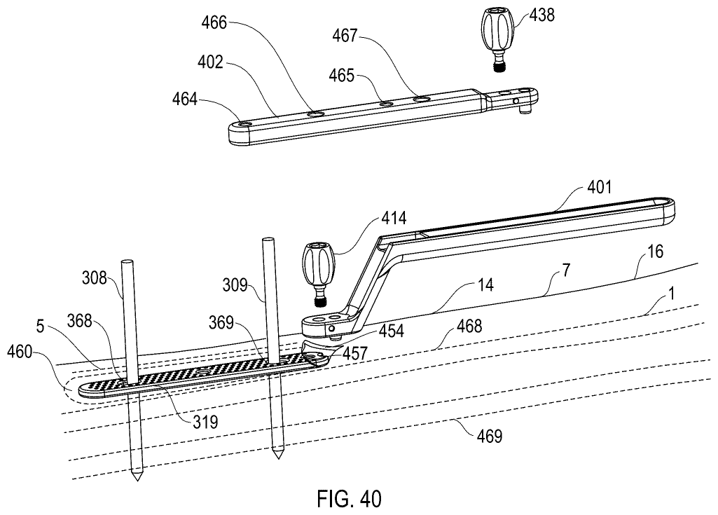

With the bone anchors 308, 309 secured in the tibia 1, and passing through the holes 368, 369 of the movable stage 319, which is within the tunnel 460, the insertion tool 400 can now be detached and removed. As illustrated in FIG. 39, the user places a torquing tool 472 through the access hole 467, which is aimed at the proximal screw drive 416 of the tightening screw 414. By turning the tip of the torquing tool in a keyed relationship with the screw drive 416, the user is able to substantially loosen or completely loosen and detach the tightening screw 414 from the internal thread 427 of the movable stage 319. Turning to FIG. 40, the user then loosens the tightening screw 438 and removes it and the targeting arm 402 from the stage-holding arm 401. The user also removes the tightening screw 414 and the stage-holding arm 401. The bone anchors 308, 309 and the movable stage 319 are now in their desired positions, the bone anchors 308, 309 in a static engagement with the bone (tibia 1) and the movable stage 319 translatable over the bone anchors 308, 309 via its holes 368, 369, to distract and stretch the periosteum 5 in relation to the bone 1, with at least a transverse component of motion.

Turning to FIG. 41, the user readies the base 301 of the PD device 300 for attachment to the bone anchors 308, 309 and the movable stage 319 (via the leadscrew 320) by assuring that the set screws 312, 313 are loosened. The user aligns hole 362 of the base 301 of the PD device 300 with a top end 473 of the bone anchor 309, aligns hole 361 of the base 301 of the PD device 300 with a top end 474 of the bone anchor 308, and inserts or aligns the distal cylindrical projection 386 of the leadscrew 320 for passage through the puncture 471 in the skin 14 of the patient 16. While pushing the leadscrew 320 through the puncture 471, the user slides the base 301 down so that the lower surface 305 of the base 301 approaches the skin 14. As mentioned, they can contact each other, or have a spec between them, or a soft material, such as gauze (cotton 4×4, etc.). The user places a torque tool into a screw drive 475 in the head 321 of the leadscrew 320 and turns the leadscrew 320 in a first direction that engages with the internal thread 379 of the movable stage 319 to place the PD device 300 in the position of FIG. 31, or a further distracted position that represents a starting position. Finally, with the base 301 in the desired position and the leadscrew 320 engaged with the movable stage 319, the user tightens a set screw 313 (as shown in FIG. 42) to engage the base 301 with the bone anchor 309. The user also does this with the set screw 312 to engage the base 301 with the bone anchor 308 (shown in process in FIG. 42). The PD device 300 is now ready to distracting and stretching the periosteum 5 by either manual rotation of the leadscrew 320, or by attachment of a modular drive unit 102 for automatic distraction. Hole 115′ is similar to hole 115 of the base 201, and female threaded hole 109′ is similar to female threaded hole 109 of the base 201 (FIG. 7), and both holes operate similarly for coupling with the modular drive unit 102. Wax can be placed into the screw drive 475 to protect its interior form dirt or other soiling until a distraction procedure. The periosteum 5 and skin 14 can now be sutured at the incisions 457, 454.

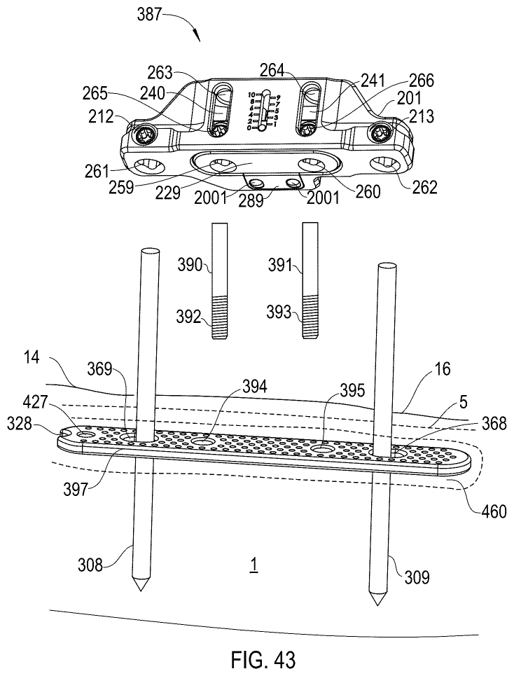

FIG. 43 illustrates an alternative periosteal distraction device (PD device) 387 that utilizes the base 201 of the transverse tibia transport device (TTT device) 200 along with the bone anchors 308, 309 and a movable stage 397, that is similar to the movable stage 319, but includes a different threaded hole configuration. The bone anchors 308, 309 and the movable stage 397 are shown in FIG. 43 after having been implanted in a patient 16 using the procedures described in relation to FIGS. 33-38. Hole 261 in the base 201 is configured to be placed over the proximal end of bone anchor 308 and secured via set screw 212. Hole 262 in the base 201 is configured to be placed over the proximal end of bone anchor 309 and secured via set screw 213. However, the movable stage 397 for distraction/stretching of the periosteum 5 is coupled to the movable stage 229 via dynamic screws 390, 391. The screws 390, 391 include distal threaded portions 392, 393 that are configured to threadingly secure to internal threadings 394, 395 that pass through the movable stage 397. The screws 390, 391 are placed into holes 259, 260 of the movable stage 229, respectively, and are secured via the set screws 265, 266, respectively. The normal translation of the movable stage 229 of the TTT device 200 is thus configured to move in unison with the movable stage 397 (plate). The TTT device 200 has thus been transformed into a periosteal distraction device (PD device) 387.

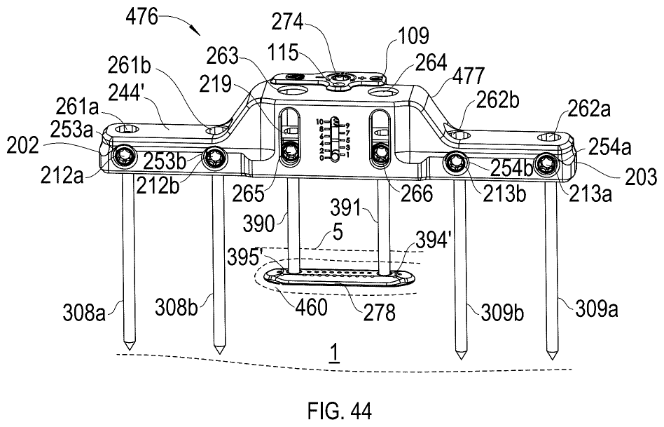

FIG. 44 illustrates an alternative periosteal distraction device (PD device) 476 that utilizes a base 477 that is similar to the base 201 of FIGS. 9 and 43, except that a first reduced-thickness end portion 244′ and a second reduced-thickness portion 245′ are further longitudinally elongated, such that they each provide two holes for static bone anchors 308a-b or 309a-b. The first reduced-thickness end portion 244′ includes holes 261a, 261b and the second reduced-thickness end portion 245′ includes holes 262a, 262b. Furthermore, the first reduced-thickness end portion 244′ comprises two female-threaded holes 253a, 253b configured to threadingly engage male threads 255a, 255b of two set screws 212a, 212b. And, the second reduced-thickness end portion 245′ comprises two female-threaded holes 254a, 254b configured to threadingly engage male threads 256a, 256b of two set screws 213a, 213b. The movable stage 478 (distraction plate) has a relatively short length in comparison to the base 477 (e.g., between 20 mm and 60 mm, or between 30 mm and 50 mm or about 40 mm). The relatively short length allows the distraction plate 478 to fit between the static bone anchors 308a-b, 309a-b, and thus not require the clearance holes 368, 369 of the movable stage plate 397 of FIG. 43. The internal threadings 394′, 395′ are similar to the internal threadings 394, 395 of the movable stage plate 397 of FIG. 43, and are configured to be threadingly secured to the distal threaded portions 392, 393 of the dynamic screws 390, 391.

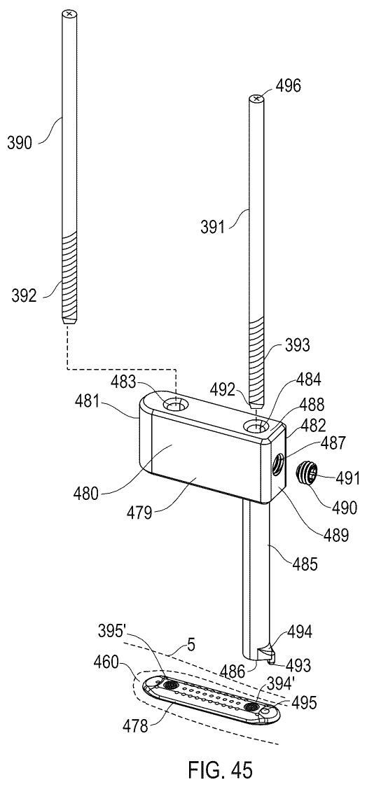

Turning to FIG. 45, a targeting tool 479 is used, instead of the targeting arm 402, to target, pass, and secure the dynamic screws 390, 391 to the internal threadings 394′, 395′ of the movable stage 478. The targeting tool 479 comprises a longitudinally-extending body 480 having a first end 481 and a second end 482. At the first end 481 is a transversely-extending through hole 483 configured to closely pass one of the dynamic screws 390, and at the second end 482 is a transversely-extending through hole 484 that is configured to closely pass the other of the dynamic screws 391, and extends further through an extension tube 485 and exits out an end exit 486. Perpendicular to the hole 484, a threaded hole 487 passes completely through a wall 488 between a side face 489 of the longitudinally-extending body 480 and the hole 484. A set screw 490 having a screw drive 491 is configured to threadingly engage with the threaded hole 487 and to tighten against and secure the dynamic screw 391. In use, after the movable stage 478 has been placed into the tunnel 460, the dynamic screw 391 is inserted through the hole 484 until a distal end 492 of the dynamic screw 391 extends from the end exit 486. The set screw 490 is then tightened to the dynamic screw 391 with a torquing tool, making it static in relation to the targeting tool 479. The targeting tool 479 further includes a transversely-extending circular protrusion 493 that is radially offset from the hole 484 via a side projection 494. The movable stage 478 includes an indexing hole 495 that is configured to closely fit the diameter of the protrusion 493. In other embodiments, the protrusion 493 and the indexing hole 495 each have a common non-circular shape, configured to closely fit each other. A puncture or incision is made in the skin 14, or previous incisions or punctures are used, and the extension tube 485/dynamic screw 391 is inserted through it/them until a lead-in (e.g., tapered) portion of the distal end 492 of the dynamic screw 391 begins to engage with the internal threading 394′ of the movable stage 478 and the protrusion 493 engages with the indexing hole 495. The targeting tool 479 can be rotated accordingly to allow for engagement of both the distal end 492 portion and the protrusion 493 are in the correct positions. The user then engages a screw drive 496 of the dynamic screw 391 with a torquing tool and tightens the distal threaded portion 393 into the internal threading 394′.

The user then passes the dynamic screw 390 through the hole 483 and tightens the distal threaded portion 392 into the internal threading 395′. The previous engagement of the dynamic screw 391 with the movable stage 478 and/or the length-to-diameter ratio of the hole 483 allow the dynamic screw 391 to correctly target and engage the internal threading 395′. The user can now loosen the set screw 490 and remove the targeting tool 479. The base 477 can now be placed onto the bone anchors 308a-b, 309a-b and the dynamic screws 390, 391 and secured with set screws 212a-b, 213a-b, 265, 266, similar to the techniques described in relation to FIGS. 41-42 and FIG. 43. The PD device 476 is now ready to distracting and stretching the periosteum 5 by either manual rotation of the leadscrew 274, or by attachment of a modular drive unit 102 for automatic distraction. Wax can be placed into the non-circular cavity 295 (screw drive) to protect its interior from dirt or other soiling until a distraction procedure. The periosteum 5 and skin 14 can now be sutured at the incisions 457, 454.

In alternative embodiments, the base 201, 301, 477 and stage 219, 229 are configured to be low profile, and can be implanted beneath the skin. In these embodiments, a hole is created through the skin the access the head 281, 381 of the adjustment screw (e.g., leadscrew 274, 320). In some embodiments, a non-invasive manner to turn the adjustment screw (e.g., leadscrew 274, 320) is contemplated, for example, with magnetic coupling or electromagnetic coupling. Furthermore, the distraction or other forced movement of a bone piece in relation to the bone can be utilized for distraction osteogenesis (DO) and/or distraction histogenesis (DH).

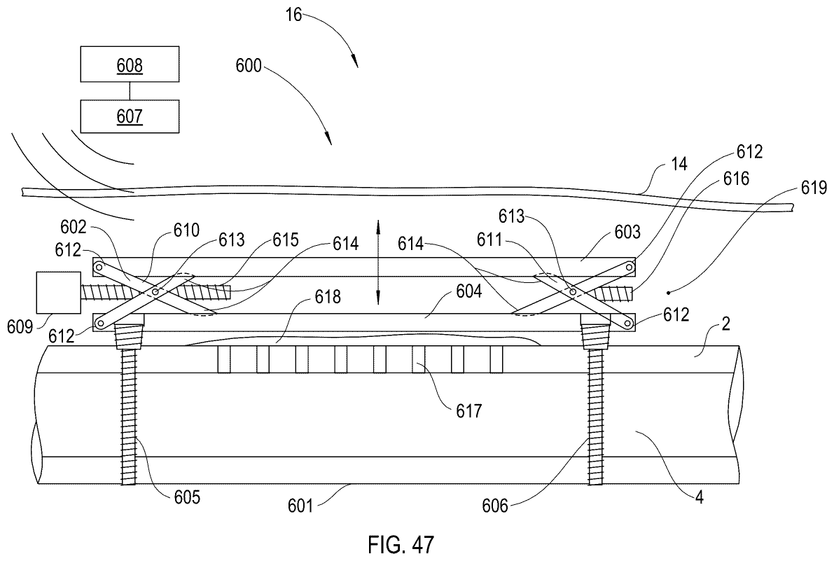

FIG. 47 illustrates an implantable device 600 comprising an internal actuator and movement mechanism 602 attached to a bone 601 comprising a cortex 2 and bone marrow 4. The internal actuator and movement mechanism 602 is configured to gradually separate a distraction element 603 of the device 600 from a fixed plate 604 or other fixed structure. As used herein, the term “base” or “base element” can include rails, channels, plates, housings, frames, and other structures, solid or non-solid. The fixed plate 604 is coupled to the cortex 2 of the bone 601 with one or more anchors, which in this embodiment comprise bone screws 605, 606. The base generally corresponds to the originating position relative to which device movement occurs. For example, when the distraction element 603 is at its closest position relative to the fixed plate 604. A non-invasive driver/power source 607 is operated by a controller 608 and is configured to non-invasive cause an internal actuator 609 to actuate, moving the distraction element 603 in relation to the fixed plate 604. The distraction element 603 is dynamically coupled to the fixed plate 604 via scissors mechanisms 610, 611. The scissors mechanisms 610, 611 include first ends 612 that are coupled to the fixed plate 604 and distraction element 603 by hinged joints 613. The scissors mechanisms 610, 611 further include sliding ends 614 that are configured to slide longitudinally in relation to the fixed plate 604 and distraction element 603 as the X-shaped scissors mechanisms 610, 611 open, causing the distraction element 603 to move (e.g., upward) away from the fixed plate 604. The driver/power source 607 non-invasively drives the internal actuator 609 to rotate one or more screws 615, 616 that move the scissors mechanisms 610, 611 in an opening direction. In some embodiments, the driver/power source 607 non-invasively is also configured to drive the internal actuator 609 to rotate screws 615, 616 in opposite directions, to move the scissors mechanisms 610, 611 in a closing direction. In some embodiments, one or both of the mechanism 602 and/or the driver/power source 607 for activation and control of the movement mechanism 602 is/are located externally and connected wirelessly to the device 600.

The entire device can be implanted, or can be attached to the bone 601 by percutaneous fasteners (e.g., the bone screws 605, 606). In some embodiments, a control for an implanted mechanism can be connected via a percutaneous wire. The controller 608 can be separately provided to manage the driver/power source 607, or the driver/power source 607 and controller 608 can be integrated into a single unit having programmed setting for operating the device 600. In use, transcortical bore holes 617 are drilled in the cortex 2 to provide a nidus for growth of regenerate bone. Transverse movement of the distraction element 603 over time, in relation to the fixed plate 604 (and thus in relation to the bone 601) causes the growth of regenerate tissue 618 within the subcutaneous tissue 619. The transverse movement can comprise upward and/or downward movement (double-headed arrow).

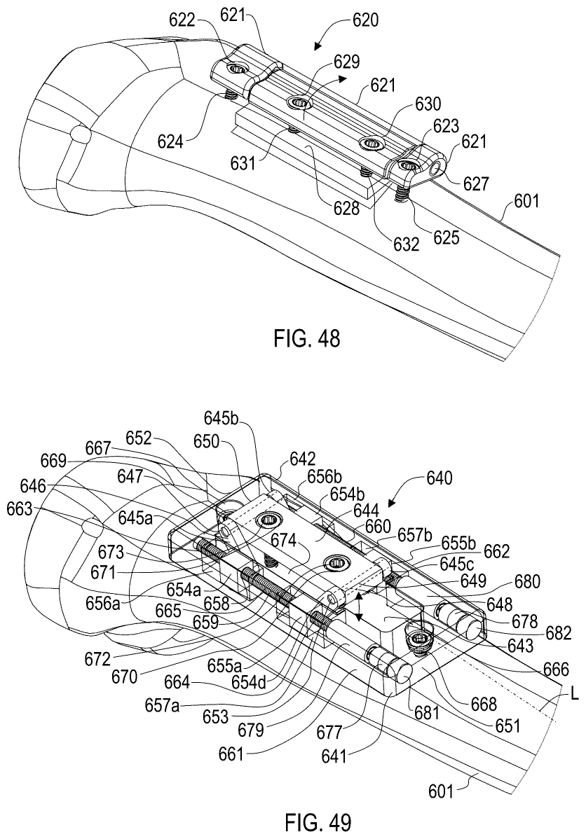

In the device 600 of FIG. 47, perpendicular motion between the elements is indicated, however, in some other embodiments, in addition to or instead of perpendicular movement, a hinge-type opening angle actuation can also achieve distraction osteogenesis as shown in FIG. 48. In FIG. 48, a device 620 comprises a base 621 comprising counterbore screw holes 622, 623 configured for placement of bone screws 624, 625 configured to lock the base 621 to the bone 601. A plate-like hinged portion 626 is rotatably coupled to the base 621 via a first hinge 627 at a first side of the base 621 and a second hinge (not shown, at the opposite side of the base 621). The hinged portion 626 comprises counterbore screw holes 629, 630 for placement of bone screws 631, 632. The bone screws 631, 632 are placed through the holes 629, 630 by the user, to screwingly engage with a bone piece 628 that is cut from the bone 601. The actuation of an internal mechanism (not shown) comprising a motor or an externally-actuated magnet, causes the rotational displacement of the bone piece 628 from the bone 601 (arrow).

The following clause includes an example of an apparatus of the disclosure:

Clause A1: A transport device for increasing biological activity within a patient, including: a base including a first end, a second end, an upper surface located between the first end and the second end, and a lower surface located between the first end and the second end; a base anchor configured to statically couple the base to a first portion of a bone of a subject; a stage pivotably coupled to the vase and configured to couple to a growth stimulator via a translatable anchor; and a drive configured to cause the stage to displace at an angle from the base.

The quality of the regeneration that occurs during distraction osteogenesis can be influenced by rate and rhythm of distraction. For example, 1 mm per day divided into ten 0.1 mm increments can in some cases produce better regeneration than two 0.5 mm increments. The medical professional can prescribe a distraction regimen that balances convenience and ability for compliance of the patient with the care of the regenerate bone. The external device provides automated distractions so that the patient does not need to worry about manual adjustments. This can alternatively be performed by an internal, implanted device. The mechanisms of action for moving the articulating member of the construct can be varied and size constraints are not as critical as it is outside the patients, for example in standard external fixators. This allows space to also integrate electronic components.

Embodiments of the devices and methods disclosed herein include both internal and external embodiments which can include any combination of described features. Aspects of the device can be characterized by four characteristic features: (1) Movements, (2) Mechanisms, (3) Power Source, and (4) Additional Technology. Various embodiments of the device could be defined by utilizing one or more features from each of the characteristic groups described below:

(1) Movements—Description of the paths of motion in order to achieve separation (changing the distance) between the intercalary segment and the native bone for distraction:
Parallel plane translation motion of the intercalary segment with the intercalary segment plane moving away from the starting plane.
   Perpendicular Lift motion in which the intercalary segment is moved in a normal direction from the reference plane to achieve distraction.
   Sliding Translation motion in which the intercalary segment plane is kept parallel but the motion path could be in a linear vector that is not perpendicular to the starting plane, or motion along an arc in order to achieve distraction.
Angular opening.
   The intercalary plane achieves separation from the starting plane through angular displacement in order to achieve distraction. This can be achieved in an opening hinge type motion.
   The intercalary plane could also move away from the starting plane with some rotation in a non-perpendicular motion so that the two planes do not remain parallel through distraction. An example of an angular separation mechanism is shown in the device 620 of FIG. 48.

(2) Mechanisms—Different types of mechanisms can be utilized in the device in order to achieve motion of the intercalary plane away from the starting plane for distraction. Examples of such mechanisms include:

### —Bar Linkages.

FIGS. 49-53 illustrate different implementations of a bar linkage for use in embodiments of the inventive device. In FIG. 49, a distraction device 640 comprises a base 641 having a rectangular shape and comprising a first end 651 and a second end 652. In other embodiment, the base 641 can have an oval, elliptical, or square shape. The base 641 has an outer frame portion 642 and an inner open portion 643. A stage 644 is moveably coupled to the base 641 by four linkages 645a-d. Each linkage 645 comprises an elongate shaft 646 and two transverse holes 647, one on each end of the shaft 646. The stage 644 includes two transverse holes 648, one adjacent to a first end 649 and one adjacent to a second end 650. Two pins 653, one toward each end 649, 650, pass through the upper transverse holes 647 of the linkages 645 and through the transverse holes 648, 649 of the stage 644 and allow free relative rotational movement (e.g., pivoting) between the linkages 645 and the stage 644. At the opposite (e.g., lower) ends of the linkages 645, the transverse holes 648 also allow the passage of pins (not shown), and these pins engage four rectangular longitudinally-translatable nuts 654a, 654b, 655a, 655b. The linkages 645 thus also have rotational freedom in relation to the nuts 654, 655.

The nuts 654a, 654b are configured to longitudinally slide within elongate cavities 656a, 656b in the base 641. The nuts 655a, 655b are configured to longitudinally slide within elongate cavities 657a, 657b. Each nut has a longitudinal female threaded interior 658 passing therethrough. Two leadscrews 659, 660 are rotatably carried by the base 641 at a first side 661 of the base 641 and a second side 662 of the base 641, respectively. In the embodiment of FIG. 49. The leadscrews 659, 660 each have turnbuckle threading, wherein a first length 663 of the leadscrew 659, 660 is configured to threadingly engage with the threaded interior 658 of the nuts 654, each having left-handed threading; and a second length 664 of the leadscrew 659, 660 is configured to threadingly engage with the threaded interior 658 of the nuts 655, each having right-handed threading. The leadscrews 659, 660 transition from the left-handed threading to the right-handed threading at a transition point 665, that is configured to remain between the longitudinal cavities 656, 657, and thus to never engage with the threaded interiors 658.

The base 641 includes two counterbore holes 666, 667 passing transversely therethrough, and configured for passage of bone screws 668, 669 for engagement with the bone 601. The stage 644 includes two counterbore holes 670, 671 passing transversely therethrough, and configured for passage of bone screws 672, 673 for engagement with a distractable bone piece 674. Thus, movement of the leadscrews 659, 660 in a first rotational direction causes the nuts 654a-b to longitudinally slide toward the second end 652 of the base 641 while also causing the nuts 655a-b to longitudinally slide toward the first end 651 of the base 641. These diverging movements cause separating longitudinal movements of the bottom ends of the four linkages 645a-d, which causes the effective height of each linkage 645 to increase, moving the stage 644 transversely away from the base 641, and thus moving the bone piece 674 upward, away from the bone 601. The movement of the leadscrews 659, 660 in a second rotational direction, opposite the first rotational direction, causes the nuts 654a-b to longitudinally slide away from the second end 652 of the base 641 while also causing the nuts 655a-b to longitudinally slide away from the first end 651 of the base 641. These converging movements cause converging longitudinal movements of the bottom ends of the four linkages 645a-d, which causes the effective height of each linkage 645 to decrease, moving the stage 644 transversely toward the base 641, and thus moving the bone piece 674 downward, toward the bone 601.

Magnetic modules 677, 678 are coupled to first ends 675, 676 of the leadscrews 659, 660, respectively, via planetary gearing 679, 680. The magnetic modules 677, 678 are longitudinally held by bushings 681, 682, or alternatively by thrust bearings. The magnetic modules 677, 678 each comprise two hollow cylindrical casings having one open end and one closed end, which are placed over opposite ends of a cylindrical magnet that is radially-poled, to protect the magnet. The thin-walled casings can comprise a non-magnetic material such as titanium and can be bonded by epoxy to the magnets. The base 641, stage 644 and all of the components are configured to be implanted underneath the skin of the patient. Thus, after recovery of the patient from implantation surgery, one or more externally-applied rotating magnetic field can be applied in the location of the magnetic modules 677, 678 to cause the modules to turn in a first direction, causing the movement of the leadscrews 659, 660 in a first rotational direction to move the stage 644 transversely away from the base 641, and thus move the bone piece 674 upward, away from the bone 601. In one embodiment, two rotating permanent magnets or two electromagnetic rotating magnetic fields are applied, each one applied at an outer position in relation to one of the magnetic modules 677, 678. The two rotating magnetic fields would be rotated in the same rotational directions as each other. Thus, two identical leadscrews 659, 660 could be turned in the same rotational direction, as described, to move the stage 644 transversely away from the base 641, and thus move the bone piece 674 upward, away from the bone 601. Alternatively, a single externally-applied rotating magnetic field would be applied at the longitudinal centerline L of the base 641, thus causing the leadscrews 659, 660 to rotate in the same rotational direction as each other.

In an alternative embodiment, the leadscrews 659, 660 can be constructed such that one leadscrew 659 has opposite threading from the other leadscrew 660 at each of the first length 663 and the second length 664. Thus, the leadscrew 659 would have left-handed threading at its first length 663 while the leadscrew 660 would have right-handed threading at its first length 663. And furthermore, the leadscrew 659 would have right-handed threading at its second length 664 while the leadscrew 660 would have left-handed threading at its second length 664. The threading 658 of the nuts 654, 655 would be similarly changed to match the leadscrew threading. In this alternative embodiment, two rotating permanent magnets or two electromagnetic rotating magnetic fields are applied, each one applied at an outer position in relation to one of the magnetic modules 677, 678. The two rotating magnetic fields would be rotated in opposite rotational directions from each other. Causing the leadscrews 659, 660 to rotate in opposite rotational directions from each other. However, because of the reverse thread directions of the two leadscrews 659, 660 and nuts 654, 655, the longitudinal movement of the nuts 659, 660 would match that of the previous embodiments. For example, either longitudinally diverging or longitudinally converging. The transverse movement of the stage 644 can comprise upward and/or downward movement (double-headed arrow).

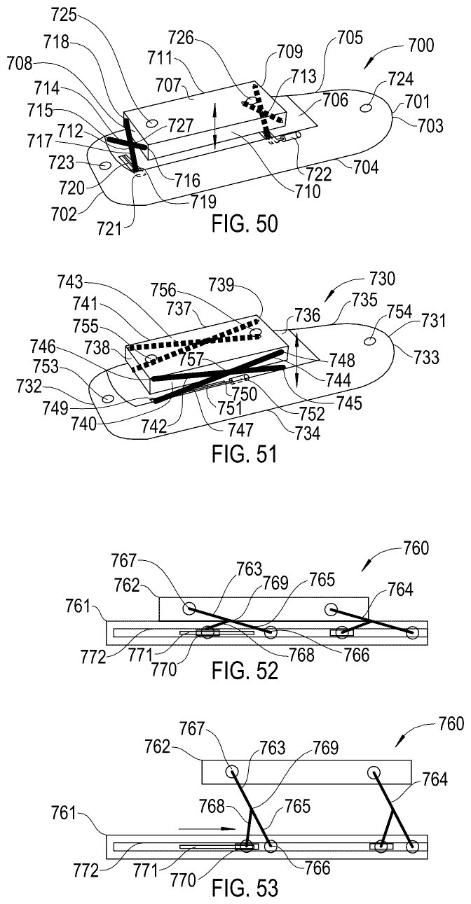

FIG. 50 illustrates a device 700 comprising bar linkage mechanisms for achieving motion of the intercalary plane away from a starting plane for transverse distraction. The device comprises a base 701 having a first end 702 and a second end 703, and a first side 704 and a second side 705. The base 701 comprises a top surface 706 comprising a flat surface. In other embodiments, the top surface 706 can be replaced with an indentation or a cavity. A stage 707, having a first end 708, a second end 709, a first side 710, and a second side 711, is transversely distractable (double-headed arrow) in relation to the base 701. First linkage 712 and second linkage 713 have some similarity to the scissors mechanisms 610, 611 (scissors expanders) of FIG. 47, however, they are coupled to the base 701 and the stage 707 at the first ends 702, 708 and the second ends 703, 709. Furthermore, each of the linkages 712, 713 include a first beam 714 that is coupled to the base 701 at a first hinged joint 715 and coupled to the stage 707 at a second hinged joint 716; and a second beam 717 that is coupled to the stage 707 at a third hinged joint 718 and coupled to the base 701 via a sliding nut 719 configured to threadingly couple to a lead screw 720 within a groove 721. Each lead screw 720 is powered by one gearmotor 722, and coupled to the gearmotor 722 by gearing, for example a bevel gear set or a worm gear set. In some embodiments, a single motor drives both leadscrews 720. In some embodiments, the first beam 714 and the second beam 717 are not coupled to each other but are configured to slide over each other or to have a small space between them. In other embodiments, the first beam 714 and the second beam 717 are pivotably coupled to each other at a hinge joint 727. The base 701 comprises holes 723, 724 for coupling to a bone with bone screws or other anchors. The stage 707 comprises holes 725, 726 for coupling to a bone piece with bone screws or other anchors, or coupling to another type of growth stimulator. Alternatively, the stage 707 is configured to distract periosteum, from a tunnel made beneath the periosteum.

FIG. 51 illustrates a device 730 comprising bar linkage mechanisms for achieving motion of the intercalary plane away from a starting plane for transverse distraction. The device comprises a base 731 having a first end 732 and a second end 733, and a first side 734 and a second side 735. The base 731 comprises a top surface 736 comprising a flat surface. In other embodiments, the top surface 736 can be replaced with an indentation or a cavity. A stage 737, having a first end 738, a second end 739, a first side 740, and a second side 741, is transversely distractable (double-headed arrow) in relation to the base 731. First linkage 742 and second linkage 743 have some similarity to the scissors mechanisms 610, 611 of FIG. 47, however, they are coupled to the base 731 and the stage 737 at the first sides 734, 740 and the second sides 735, 741. Furthermore, each of the linkages 742, 743 include a first beam 744 that is coupled to the base 731 at a first hinged joint 745 and coupled to the stage 737 at a second hinged joint 746; and a second beam 747 that is coupled to the stage 737 at a third hinged joint 748 and coupled to the base 731 via a sliding nut 749 configured to threadingly couple to a lead screw 750 within a groove 751. Each lead screw 750 is powered by one gearmotor 752, and coupled to the gearmotor 752 by gearing, for example a bevel gear set or a worm gear set. In some embodiments, a single motor drives both leadscrews 750. In some embodiments, the first beam 714 and the second beam 717 are not coupled to each other but are configured to slide over each other or to have a small space between them. In other embodiments, the first beam 744 and the second beam 747 are pivotably coupled to each other at a hinge joint 757. The base 731 comprises holes 753, 754 for coupling to a bone with bone screws or other anchors. The stage 737 comprises holes 755, 756 for coupling to a bone piece with bone screws or other anchors, or coupling to another type of growth stimulator. Alternatively, the stage 737 is configured to distract periosteum, from a tunnel made beneath the periosteum.

FIGS. 52 and 53 illustrate an alternative device 760 comprising bar linkage mechanisms for achieving motion of the intercalary plane away from a starting plane for transverse distraction. The device 760 comprises a base 761 and a stage 762 coupled to each other by two side linkages 763, 764 per side (visible only on one side in FIGS. 52 and 53). Each of the linkages 763, 764 include a first beam 765 that is coupled to the base 761 at a first hinged joint 766 and coupled to the stage 762 at a second hinged joint 767; and a second beam 768 that is pivotably coupled to a center portion of the first beam 765 at a third hinged joint 769 and coupled to the base 761 via a sliding nut 770 configured to threadingly couple to a lead screw 771 within a groove 772. Each lead screw 771 is powered by one gearmotor (not shown), and coupled to the gearmotor by gearing, for example a bevel gear set or a worm gear set. In some embodiments, a single motor drives multiple leadscrews. The base 761 comprises holes for coupling to a bone with bone screws or other anchors. As the sliding nut 770 is moved longitudinally (arrow), the pivoting of the linkages 763, 764 moved the stage 762 transversely (e.g., upward). The stage 762 comprises holes for coupling to a bone piece with bone screws or other anchors, or coupling to another type of growth stimulator. Alternatively, the stage 762 is configured to distract periosteum, from a tunnel made beneath the periosteum.

The following clauses include examples of apparatus of the disclosure:

Clause B1: A transport device for increasing biological activity within a patient, including: a base including a first end, a second end, an upper surface located between the first end and the second end, and a lower surface located between the first end and the second end; a base anchor configured to statically couple the base to a first portion of a bone of a subject; a translatable anchor configured to engage a growth stimulator; a leadscrew dynamically coupling the base to the translatable anchor; and at least one scissors mechanism comprising a first beam pivotably coupled to a second beam,

Clause B2: The device of clause B1, wherein the first beam is pivotably coupled to the second beam with a pin.

Clause B3: The device of clause B1, wherein the first beam includes a first end pivotably coupled to the base and the second slidable end.

Clause B4: The device of clause B3, wherein the second beam includes a first end pivotably coupled to a stage that is statically coupled to the translatable anchor and a second slidable end.

### —Nut and Lead Screw.

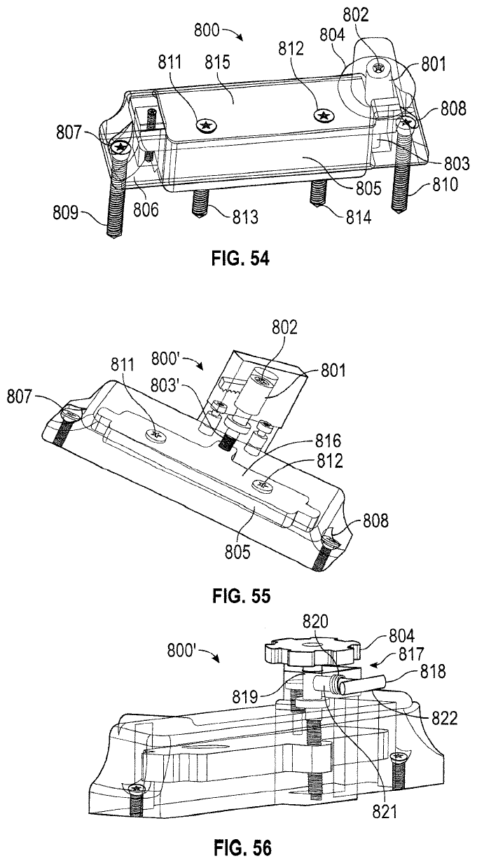

An example of a nut and lead screw assembly 800 is illustrated in FIG. 54, where a nut 801 with an internal thread 802 is embedded inside the intercalary segment/plate 805. In other embodiments, an internal thread can be machined inside the intercalary segment (on the right side of the plate). A matching leadscrew 803 is driven by a knob 804 that users can manually rotate in order to move the intercalary segment up and/or move down. The assembly 800 comprises a base 806 comprising two holes 807, 808 through which bone anchors 809, 810 (e.g., bone screws) are placed, for securing to a bone. A movable stage portion 815 comprises two holes 811, 812 through which bone anchors 813, 814 (e.g., bone screws) are placed, for securing to the intercalary segment 805.

The other side of the intercalary segment can be constrained by a pin or a block on the left end, or can be constrained by pins or blocks on the two narrow sides.

The leadscrew and nut assembly 800′, in the embodiment shown in FIG. 55, can be placed in the middle of the stage portion 815. The leadscrew 803′, can be located at the end of the assembly 800, or on a side of the assembly 800′, towards a central portion 816. The leadscrew 803, 803′ can be locked/unlocked by any of a variety of different locking mechanisms. One example of a locking mechanism 817 is a spring-loaded cylinder-shaped latch 818 that slides inside a block 819 that is fixed/screwed in the base 806 (FIG. 56). The spring 820 has a bias and is naturally under compression, and presses the tip of the latch 818 in one of the circumferential slots 821 of the leadscrew 803′. Thus, the leadscrew 803′ is normally locked; thus, the stage 815 is also locked by default. In order to rotate the leadscrew 803′ and move the stage 815, the user pulls the a handle 822 of the latch 818 out to further compress the spring 820, so that its tip is removed from the slot 821 of the leadscrew 803′ to unlock it. While the latch 818 is being pulled, the user can rotate the leadscrew 803′ to move the stage 815 up or down. Once the user releases the latch handle 822, the latch 818 automatically returns to its default state to lock the leadscrew 803′.

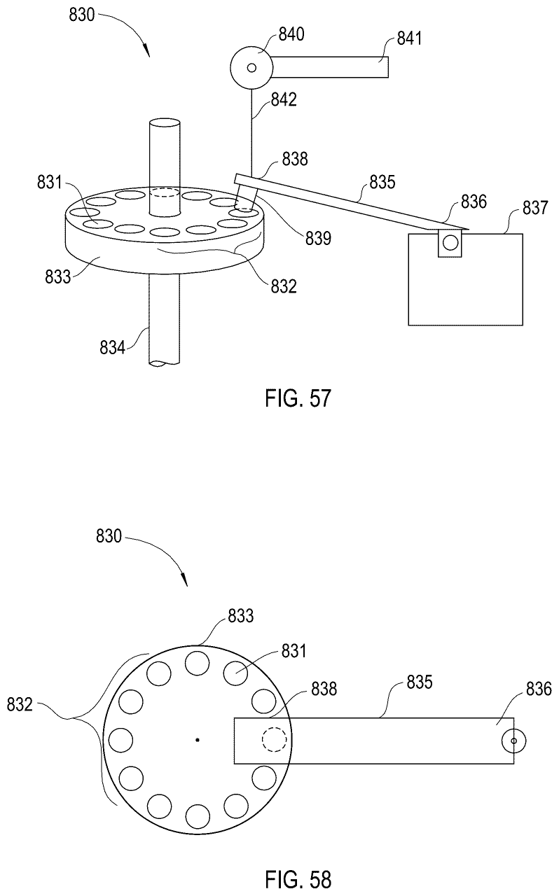

In another embodiment, illustrated in FIGS. 57-58, a locking mechanism 830 includes multiple holes 831 (twelve holes in the embodiment of FIGS. 57-58) disposed proximate to each other in a circumferential array 832 through the shoulder 833 of the leadscrew 834. A spring/leaf arm 835 has a first end 836 fixed on the base 837. On a second, distal end 838 of the arm 835, a peg 839 (or pin) extends downward, toward the shoulder 833, where it is inserted in one of the holes 831 as a result of the spring bias applied by the arm 835, causing the peg 839 to lock the leadscrew 834, preventing its rotation. In order to rotate the leadscrew 834 and move the stage/intercalary segment vertically, the user can pull a pull ring 840 or other gripping structure 841 extending from the distal end of the arm 835 (e.g., via a tether 842) to lift the pin 839 out of the hole 831. After the pin 839 is removed from the hole 831, the user is able to rotate the leadscrew 834 to move the stage/intercalary segment up or down. Once the pull ring 840 is released, the spring bias of the arm 835 causes the pin 839 to automatically move into the nearest hole 831, returning the locking mechanism (assembly) 830 to its default locked state.

### —Scotch Yoke.

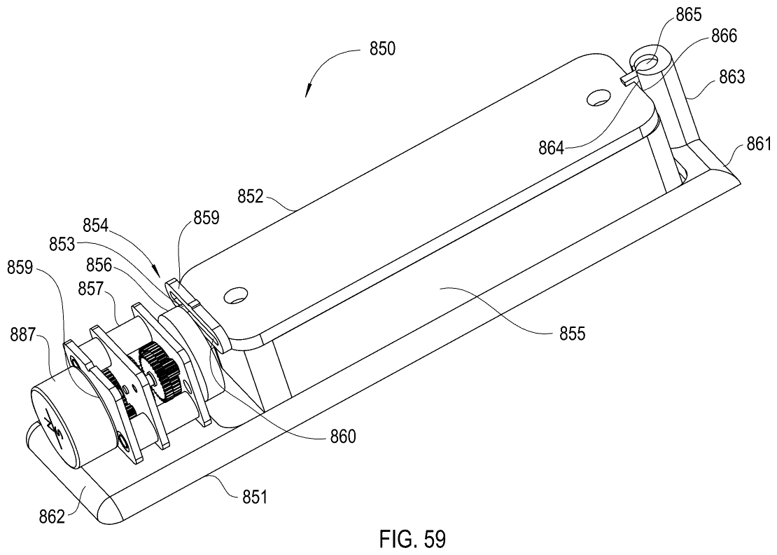

A device 850 comprising a base 851 and a transversely distractable stage 852 is shown in FIG. 59. A driver output shaft 859 (from a motor, or actuator or a manual knob) drives a scotch yoke 854 via an interconnecting gear box 857. The scotch yoke 854 is attached to the stage 852. The stage 852 is configured to be attached to a bone fragment 855. A rotator 856 is an output from the gear box 857. A pin 853 that is located at a peripheral portion of the rotator 856, extends toward the stage 852 and the bone fragment 855. The pin 853 is configured to slidably fit within a horizontally-extending slot 860 of a yoke 859 that is coupled to the stage 852. As the rotator 856 is rotated by a motor, or manually, or in this particular embodiment, a driven radially-poled permanent magnet 887, the pin 853 is able to slide within the slot 860 as the rotator 856 is rotated. The peripheral location of the pin 853 is such that as the rotator 856 is rotated, the pin 853 also lifts the stage 852, and thus lifts the bone fragment 855. At a second end 861 of the device 850, opposite the first end 862, a vertical column 863 is coupled to and extends vertically from the base 851. The vertical column 863 has an inwardly-facing vertically-extending groove 864 which slidingly fits a tab 865 that is attached to an end 866 of the stage 852. Thus, the stage 852 is balanced and is able to slide up (or down) when being driven by the scotch yoke 854.

### —Driving Wedge.

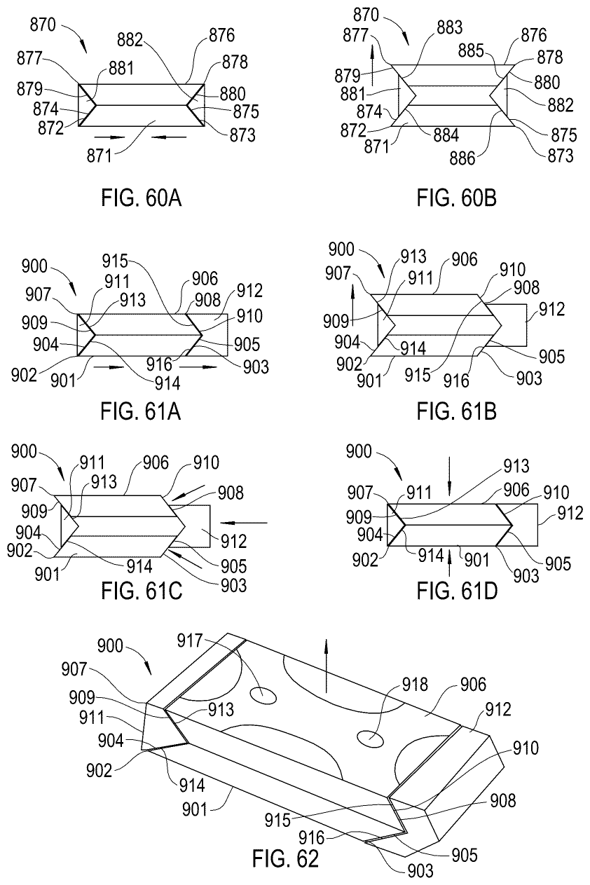

In several embodiments, a driver output shaft (from a powered actuator or a manually-driven knob) drives a wedge block via a leadscrew/nut, and the wedge of the blocks drives a stage and a bone fragment attached to the stage via a wedge or a pin. Through the wedge, the linear horizontal movement of the wedge block is transferred to vertical movement of the intercalary plate. An example of such design is shown in FIGS. 60A-60B. A wedge device 870 comprises a base portion 871 having a first end 872 and a second end 873. The first and second ends 872, 873 each comprise an angled face 874, 875 that causes the base 871 to have a shorter length at the top of the angled faces 874, 875 and a longer length at the bottom of the angled faces 874, 875. The wedge device 870 also comprises a stage portion 876 having a first end 877 and a second end 878. The first and second ends 877, 878 each comprise an angled face 879, 880 that causes the stage 876 to have a shorter length at the bottom of the angled faces 879, 880 and a longer length at the top of the angled faces 879, 880. The angled faces 874, 875, 879, 880 thus provide an inward wedge shape in a first state shown in FIG. 60A. Two triangular wedge pieces 881, 882, one at each end of the wedge device 870, are each configured to fit into the inward (concave) space provided by the angled faces 874, 875, 879, 880. As driven by a motorized actuator or a manually-driven actuator, the wedge pieces 881, 882 are driven inward (arrows), such that their angled inner faces 883, 884, 885, 886 slide against the angled faces 879, 874, 880, 875, respectively, causing the stage portion 876 to distract upward (e.g., transversely) from the base 871, as shown in FIG. 60B (vertical arrow).

Another example of such design is shown in FIGS. 61A-62. A wedge device 900 comprises a base portion 901 having a first end 902 and a second end 903. The first and second ends 902, 903 each comprise an angled face 904, 905 that causes the base 901 to have a parallelogram shape. The angled faces 904, 905 are generally parallel to each other and are not parallel to the vertical plane. The wedge device 900 also comprises a stage portion 906 having a first end 907 and a second end 908. The first and second ends 907, 908 each comprise an angled face 909, 910 that causes the stage 906 to have a parallelogram shape. The angled faces 909, 910 are generally parallel to each other and are not parallel to the vertical plane. The angulation of the angled faces 904, 905 and the angulation of the angled faces 909, 910 provide two V-shapes, one concave and one convex, in relation to the device 900. The angled faces 904, 909 thus provide an inward wedge shape and the angled faces 905, 910 provide an outward wedge shape, in a first state shown in FIG. 61A. A triangular wedge piece 911, at a first end of the wedge device 900, is configured to fit into the inward (concave) space provided by the angled faces 904, 909. A wedge piece 912 having a concave triangular shape, at a second end of the wedge device 900, is configured to fit into the outward (convex) projection provided by the angled faces 905, 910. The triangular wedge piece 911 comprises angled faces 913, 914. The wedge piece 912 comprises angled faces 915, 916. As driven by a motorized actuator or a manually-driven actuator, the wedge pieces 911, 912 are driven in the same direction as each other (arrows, FIG. 61A), while at least the base 901 is prevented from longitudinal movement, such that the angled faces 914, 916, 913, 915 slide against the angled faces 904, 905, 909, 910, respectively, causing the stage portion 906 to distract upward (e.g., transversely) from the base 901, as shown in FIG. 61B (vertical arrow). As driven for opposite directional movement by a motorized actuator or a manually-driven actuator (arrow, FIG. 61C), the process is revered and the stage portion 906 returns toward the base portion 901. As shown in FIG. 62, the stage portion 906 comprises holes 917, 918 for passage of bone anchors.

The following clauses include examples of apparatus of the disclosure:

Clause C1: A transport device for increasing biological activity within a patient, including: a base including a first end and a second end; a base anchor configured to statically couple the base to a first portion of a bone of a subject; a stage having a first end and a second end and configured to engage a growth stimulator; a first angled surface carried on the first end of the base; a second angled surface carried on the first end of the stage; a first wedge having a third angled surface configured for sliding contact with the first angled surface and a fourth angled surface configured for sliding contact with the second angled surface; and a drive configured to move the first wedge in relation to the base and the stage, wherein movement of the first wedge in a first direction causes the base and the stage to move apart from each other via simultaneous sliding of the third angled surface with the first angled surface and sliding of the fourth angled surface with the second angled surface.

Clause C2: The transport device of clause C1, further including: a fifth angled surface carried on the second end of the base; a sixth angled surface carried on the second end of the stage; and a second wedge having a seventh angled surface configured for sliding contact with the fifth angled surface and an eighth angled surface configured for sliding contact with the sixth angled surface, wherein movement of the second wedge in a second direction, opposite the first direction, causes the base and the stage to move apart from each other via simultaneous sliding of the seventh angled surface with the fifth angled surface and sliding of the eighth angled surface with the sixth angled surface.

Clause C3: The transport device of clause C1, further including: a fifth angled surface carried on the second end of the base; a sixth angled surface carried on the second end of the stage; and a wedge piece having a seventh angled surface and an eight angled surface that together form a V-shaped concavity, wherein the seventh angled surface is configured for sliding contact with the fifth angled surface and wherein the eighth angled surface is configured for sliding contact with the sixth angled surface, wherein movement of the second wedge in a second direction, opposite the first direction, causes the base and the stage to move each other via simultaneous sliding of the seventh angled surface with the fifth angled surface and sliding of the eighth angled surface with the sixth angled surface.

### —Pin in Slot.

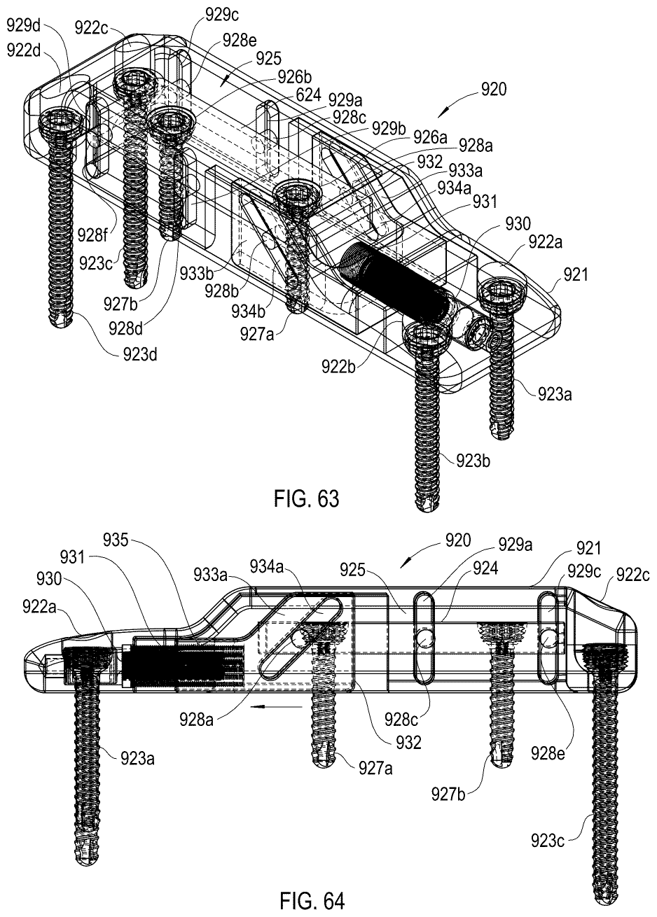

In embodiments comprising the driving of a transfer bar to push a pin to follow a track, an intercalary member can also be directly coupled to a leadscrew, or any other mechanisms that provide driving movement. Examples of this approach are shown in FIGS. 63-64. An implantable distraction device 920 comprises a base 921 having four holes 922a-d for placement of bone screws 923a-d. A stage 924 is configured to be distracted within an internal cavity 925 of the base 921. The stage 924 comprises two holes 926a-b for placement of bone screws 927a-b. The bone screws 923a-d are configured to secure to a bone, and the bone screws 927a-b are configured to secure to a bone fragment or other growth stimulator. The stage 924 includes transversely-extending pins 928a-f which can be press fit, screwed-in, or molded in place. Two pins 928a-b are at a first end, left and right, two pins 928c-d are at a middle portion, left and right, and two pins 928e-f at a second end, left and right. The base 921 includes four vertical grooves 929a-d, two on each side, each groove 929a-d configured to allow one of pins 928c-f to vertically slide, to stabilize the stage 924 are it is distracted in relation to the base 921. A motor 930 having a gearbox 931 is powered by a battery which can be configured to be rechargeable. The motor 930 is connected to a lift bracket 932 comprising a left and right vertically-extending walls 933a-b. Each wall 933a-b comprises a slanted groove 934a-b. Pins 928a-b are configured to slide within the slanted grooves 934a-b. The motor 930 and gearbox 931 output to an actuator 935 that translates rotational motion to longitudinal displacement (horizontal arrow in FIG. 64). Thus, as the motor 930 drives the horizontal motion, the grooves 934a-b are pushed against the pins 928a-b causing the stage 924 to be moved upward.

The following clauses include examples of apparatus of the disclosure:

Clause D1: A transport device for increasing biological activity within a patient, including: a base including a first end and a second end; a base anchor configured to statically couple the base to a first portion of a bone of a subject; a stage having a first end and a second end and configured to engage a growth stimulator, the stage including a first laterally-extending projection; a shuttle configured for longitudinal movement on or in the base, the shuttle including a vertically-extending wall having a slanted groove, the first laterally-extending projection configured to slidingly engage the groove; a drive configured to move the shuttle in a first longitudinal direction, wherein movement of the shuttle in the first longitudinal direction causes the first laterally-extending projection to be moved within the groove the movement including at least some upward movement, and wherein the stage is caused to be moved along with the first laterally-extending projection.

Clause D2: The transport device of clause D1, further including: a vertical groove on the vertically-extending wall configured to slidingly engage a second laterally-extending projection of the stage, wherein the second laterally-extending projection maintains a longitudinal position of the stage in relation to the base as the second laterally-extending projection is slid within the vertical groove.

### —Rack and Pinion.

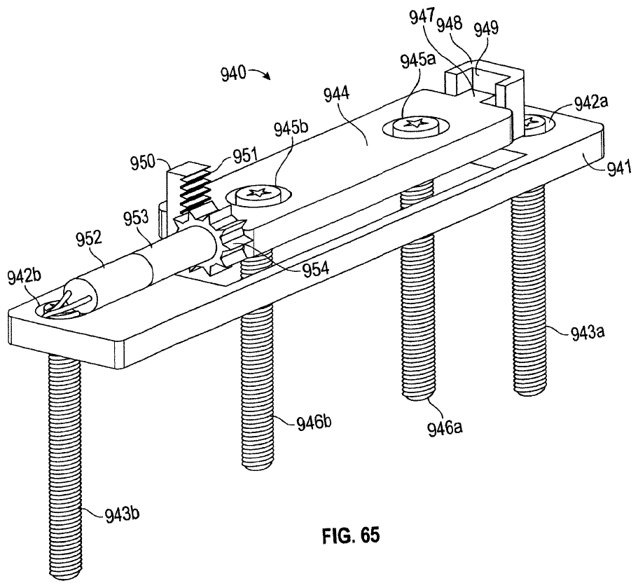

Another possible embodiment employs a pinion as the output driver that drives a rack which is attached to the stage that is screwed on the bone fragment. This approach is not limited to a single rack-and-pinion—two racks attached on each side of the intercalary plate can be driven together by the pinions on the output shaft of the flat spur gear system to increase system stability. Examples of such a design is shown in FIG. 65 (one rack). Alternatively, the design can comprise two racks (FIG. 97). The rack and pinion distraction device 940 in FIG. 65 comprises a base 941 having two holes 942a-b for placement of bone anchors 943a-b configured to be screwed into a bone. A stage 944 comprises two holes 945a-b for placement of bone anchors 946a-b configured to be screwed into a bone fragment. The stage 944 includes a longitudinal end projection 947 and the base 941 includes a vertically-extending post 948 having a vertical groove 949. The projection 947 slides vertically within the groove 949. At an opposite end of the stage 944, a rack 950 having a plurality of teeth 951 extends vertically and is statically coupled to the stage 944. A motor 952 and gearbox 953 are coupled together and held on the base 941. The gearbox 953 outputs to a shaft that is rotationally coupled to a pinion 954 (e.g., pinion gear) having teeth. Thus, as the motor is powered (e.g., by a battery) it runs the pinion 954 which is engaged with the teeth of the rack 950, moving the rack upward, and thus moving the stage 944 upward. As the stage moves updated, the bone fragment is transversely moved in relation to the bone.

A fully-implantable rack and pinion distraction device 960 in FIG. 97 comprises a base 961 having two holes 962a-b for placement of bone anchors configured to be screwed into a bone. A stage (not shown) carries the gear train 963 comprising first and second pinions 964a-b which are configured to engage with and drive the stage up an angled path in relation to first and second angled racks 965a-b, respectively. The pinions 964a-b are carried on an axle 966 that also rotationally carries a central gear 967. The central gear belongs to a gear train 968 including a second gear 969, and third gear 970, a fourth gear 971, and a worm 972. The worm 972 is held at the end of a shaft 973 and a radially-poled magnet 974 is held at the other end of the shaft 973. An external moving magnetic field 975, external to the skin 976 of the patient, can be applied in either rotational direction to turn the magnet 974 in the desired direction to distract or retract the stage in relation to the base 961. Alternative embodiments of the devices 940, 960 of FIGS. 65 and 97 can comprise the rack attached to the stage and the pinion and motor (or other drive) attached to the base, or the alternative of the rack attached to the base and the pinion and motor (or other drive) attached to the stage.

The following clauses include examples of apparatus of the disclosure:

Clause E1: A transport device for increasing biological activity within a patient, including: a base including a first end, a second end, an upper surface located between the first end and the second end, and a lower surface located between the first end and the second end; a base anchor configured to statically couple the base to a first portion of a bone of a subject; a stage configured to engage a growth stimulator; a drive coupled to the base and configured to output rotation to a pinion; and a rack coupled to the stage, wherein the pinion is configured to engage the rack and to lift the stage as the pinion is turned in a first direction.

Clause E2: A transport device for increasing biological activity within a patient, including: a base including a first end, a second end, an upper surface located between the first end and the second end, and a lower surface located between the first end and the second end; a base anchor configured to statically couple the base to a first portion of a bone of a subject; a stage configured to engage a growth stimulator; a drive coupled to the stage and configured to output rotation to a pinion; and a rack coupled to the base, wherein the pinion is configured to engage the rack and to lift the stage as the pinion is turned in a first direction.

A face-to-face arrangement between an implanted driven radially-poled permanent magnet 977 (e.g., as part of a device, like the magnet 974 in the device 960 of FIG. 97) and an external magnet 978 (outside of the skin 976) is shown in FIG. 98. Rotation of the external magnet 978 in a first rotational direction causes rotation of the implanted magnet 977 in the same direction. In FIG. 99, the external magnet 980 has a cup shape having an inner dimeter D1, that is larger than an outer diameter Do of the implanted magnet 979. The inner diameter D1 also is large enough to allow some skin 976 to be forced between the two magnets 980, 979 by pushing the external magnet 980 down over the implanted magnet 979. Thus, in this pushed-down position, the external magnet 980 drives the implanted magnet 979 not only via the face-to-face geometry of FIG. 98, but also by radial overlap, outer to inner. This increases the driving and coupling torque and allows the worm 981 on shaft 982 to drive a larger torque against other gears, which being able to distract bone fragment (or stretch periosteum) at larger forces, that may be required.

FIG. 100 illustrates an elongate cylindrical implanted, radially-poled permanent magnet 983 and an elongate cylindrical external, radially-poled permanent magnet 984. This configuration allows the magnets to work as non-contact gears. The radial poling of either magnet can comprise two, or four, or more poles. Two poles per magnet are shown in FIG. 100. In some embodiments, the poles can be unequal, for example, a four-pole magnet driving a two-pole magnet. The magnet 984 drives the magnet 983 to turn in an opposite direction, as shown by the curved arrows.

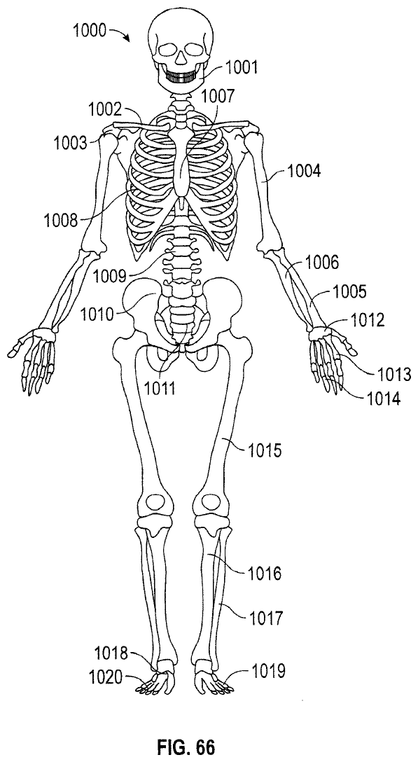

FIG. 66 illustrates a human skeleton 1000, showing some of the bones that may be treated with the devices described herein. Transverse distraction of the cortex or distraction/stretching of the periosteum can be performed in relation to any of the following bones: the mandible 1001, the clavicle 1002, the scapula 1003, the humerus 1004, the radius 1005, the ulna 1006, the sternum 1007, one or more rib 1008, one or more vertebra 1009, an ilium 1010, the sacrum 1011, one or more carpal 1012, one or more metacarpal 1013, one or more phalanges 1014 of the hand, the femur 1015, the tibia 1016, the fibula 1017, one or more tarsal 1018, one or more metatarsal 1019, and one or more phalanges 1020 of the foot. A fragment of these bones can be osteotomized and then distracted from its anatomic position.

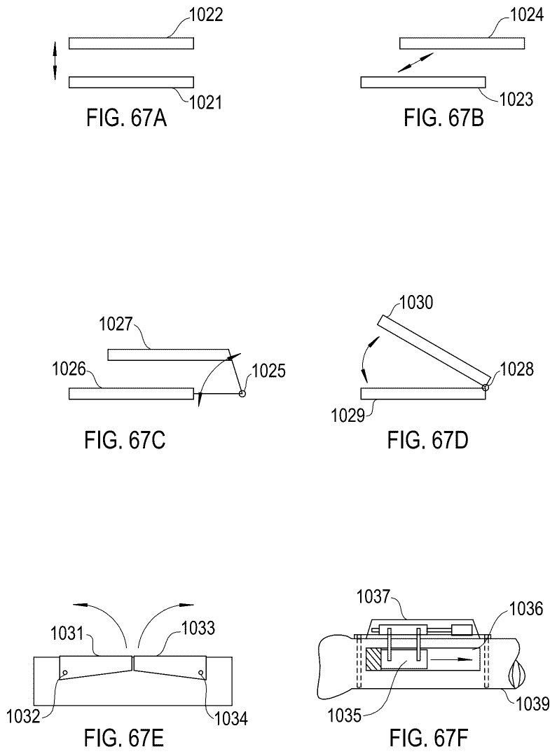

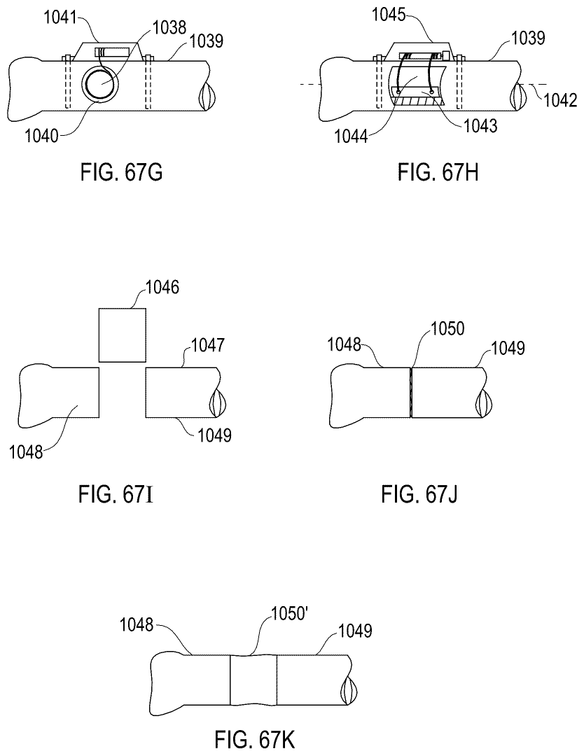

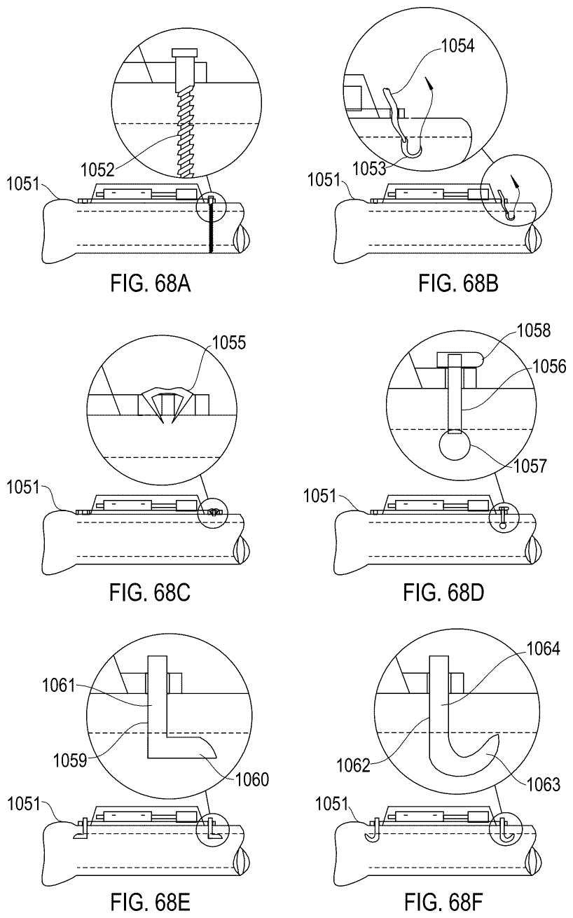

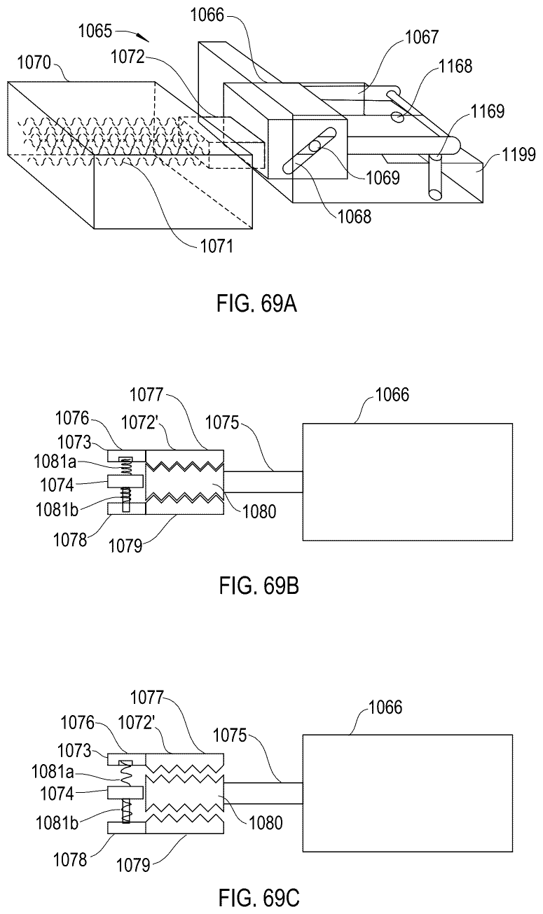

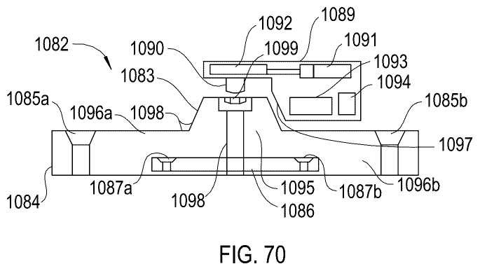

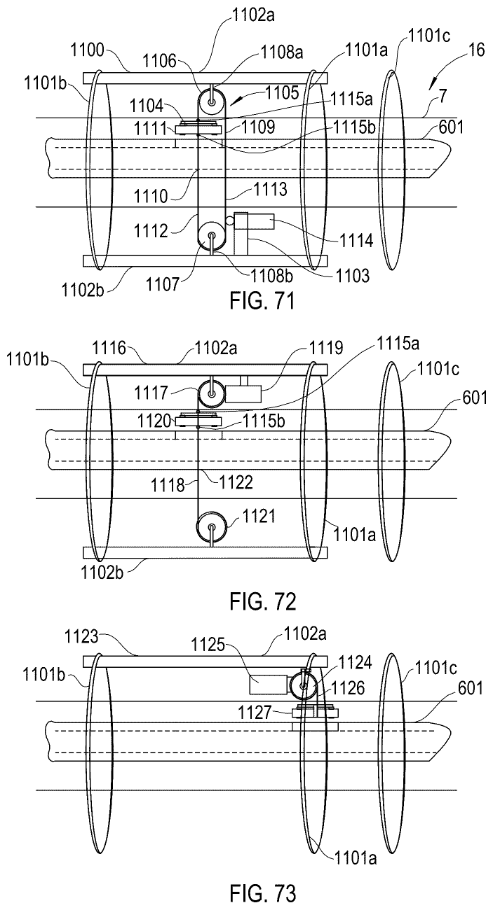

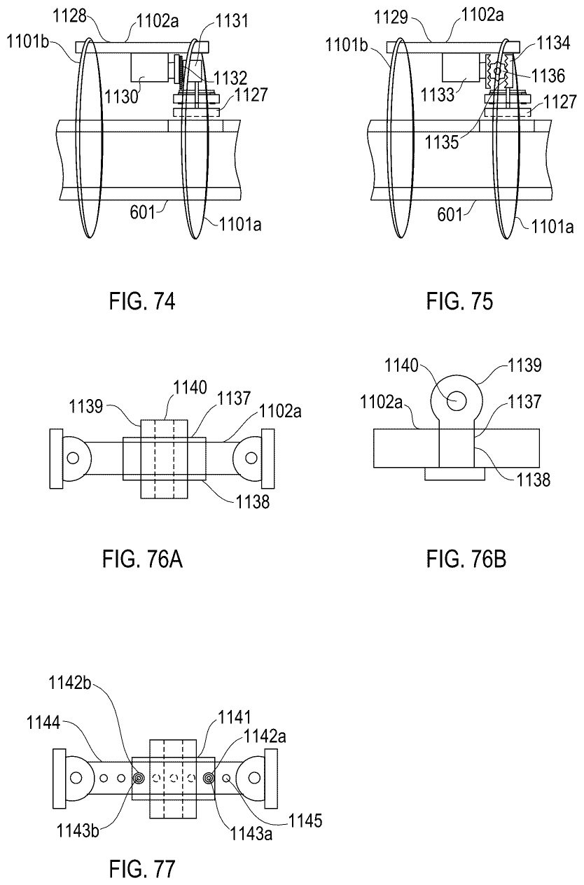

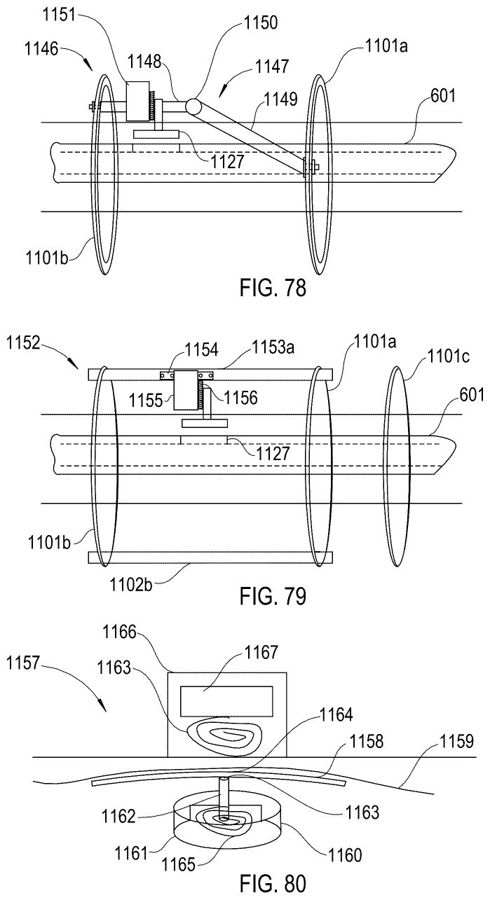

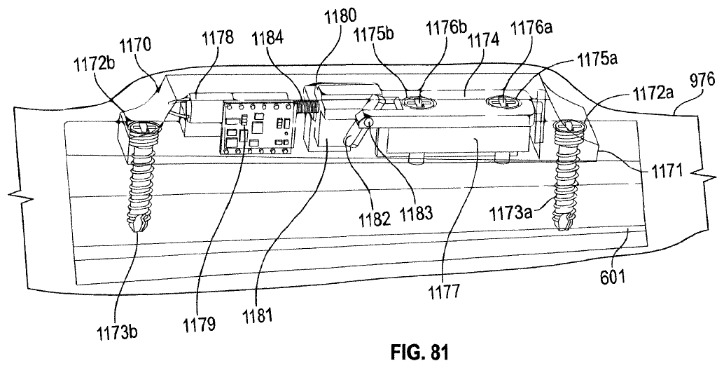

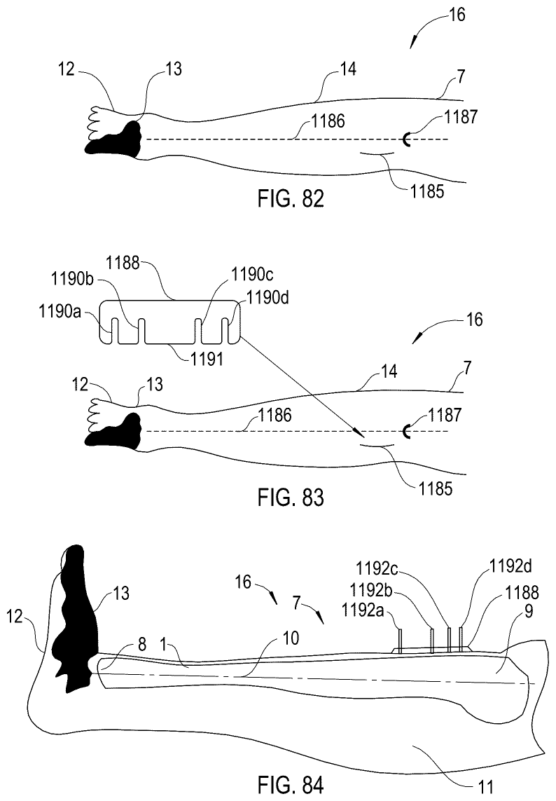

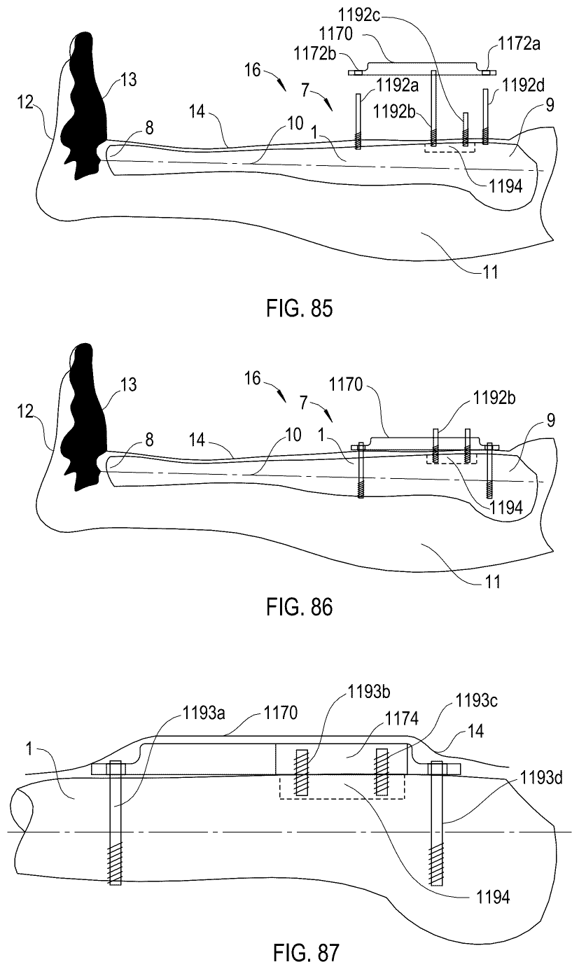

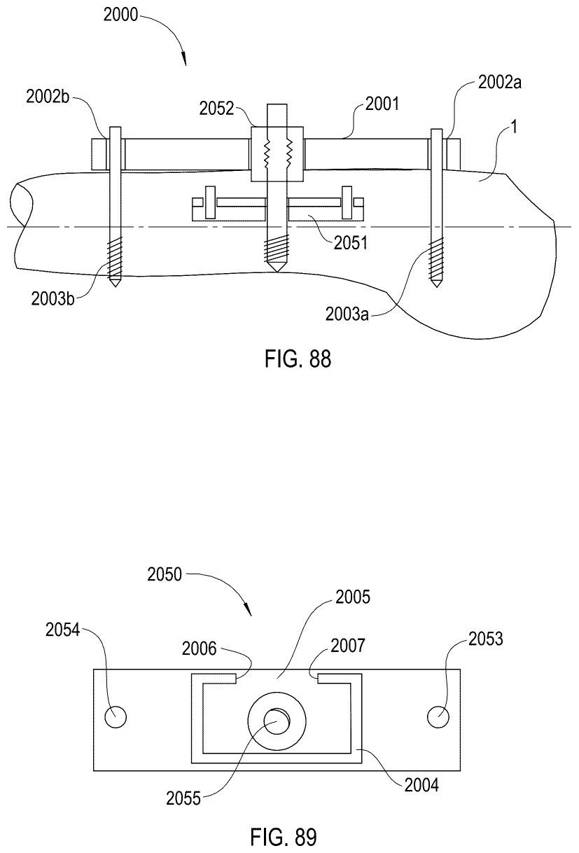

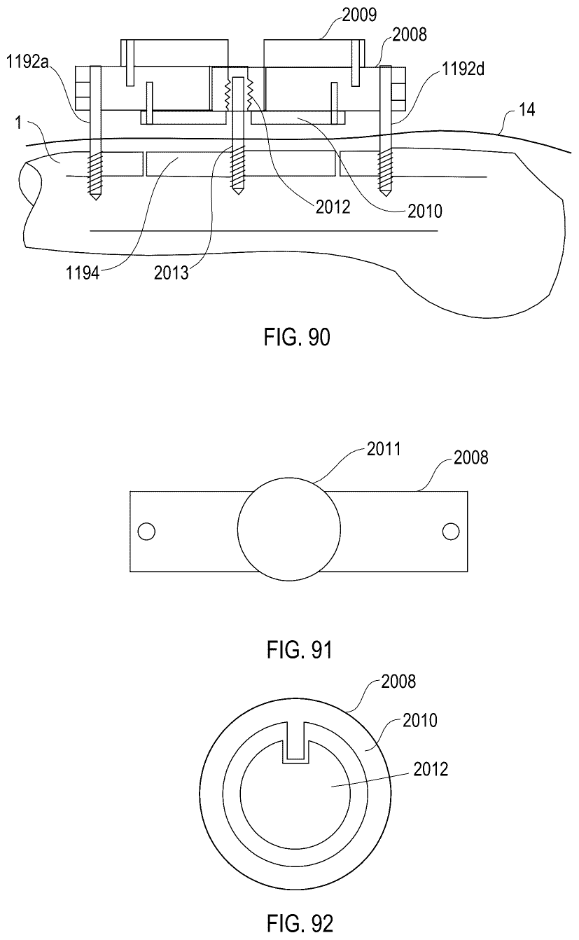

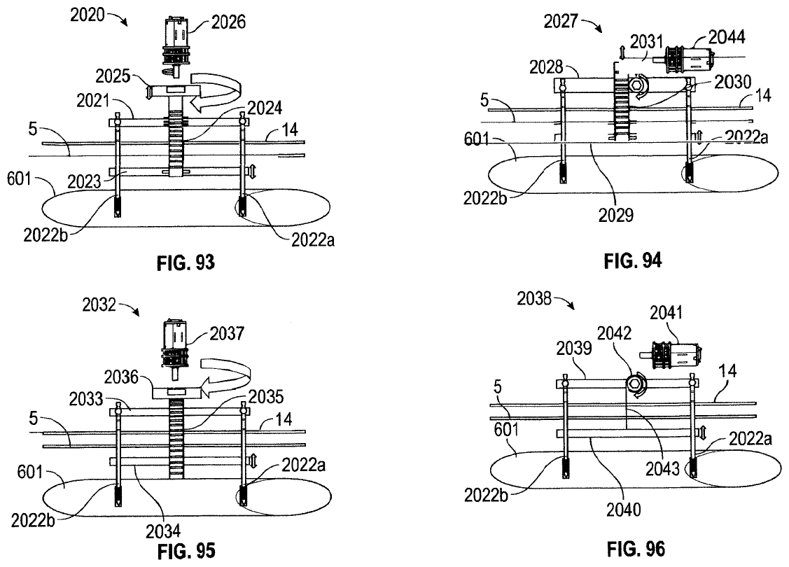

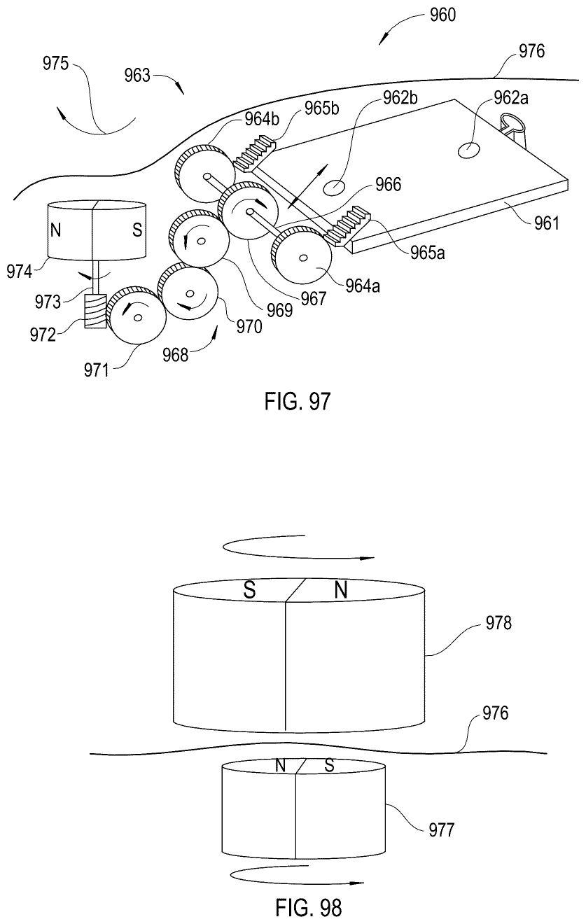

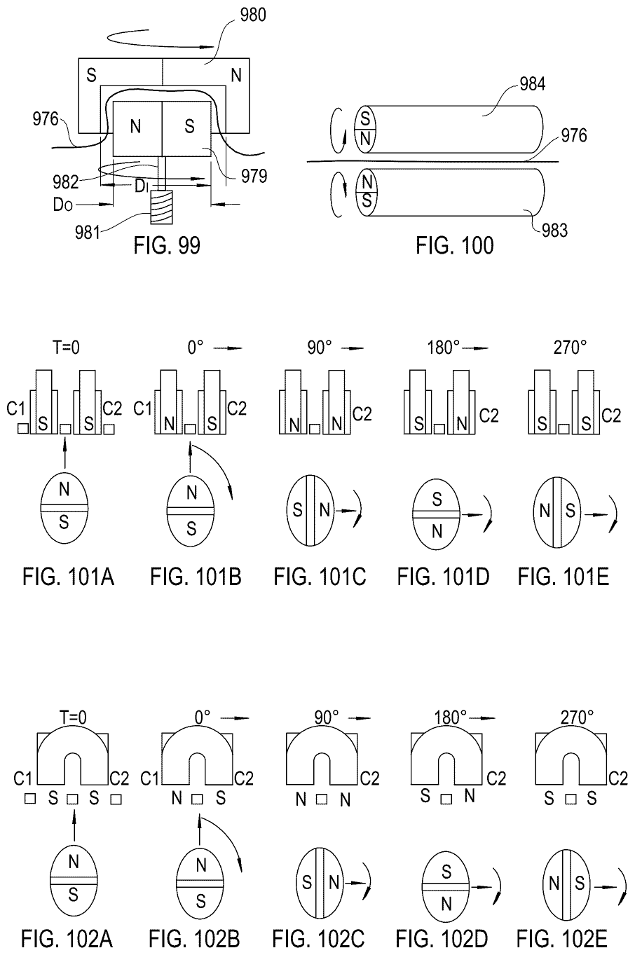

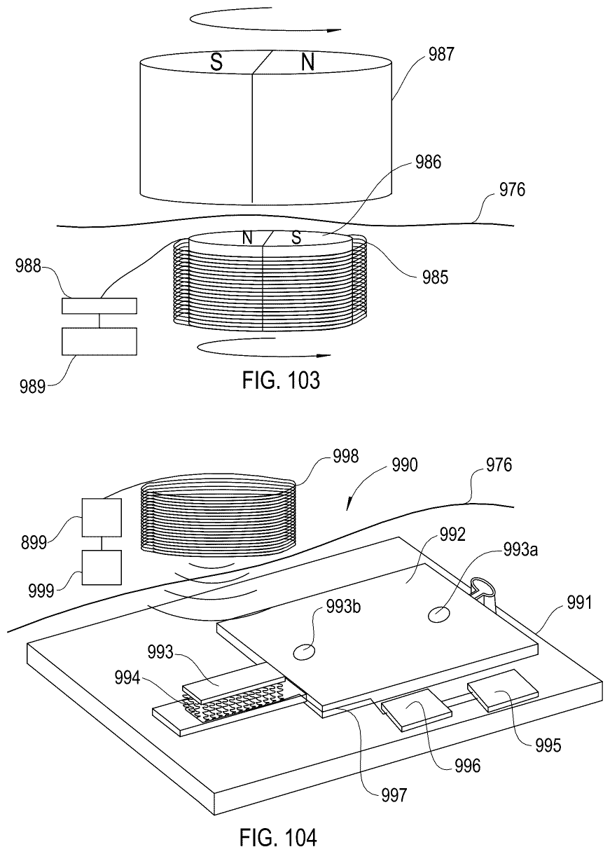

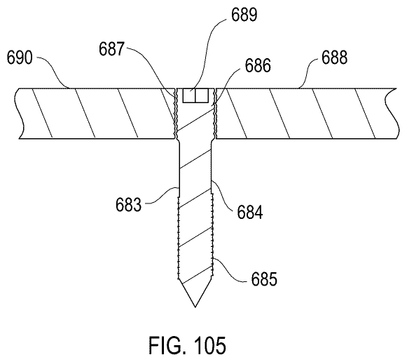

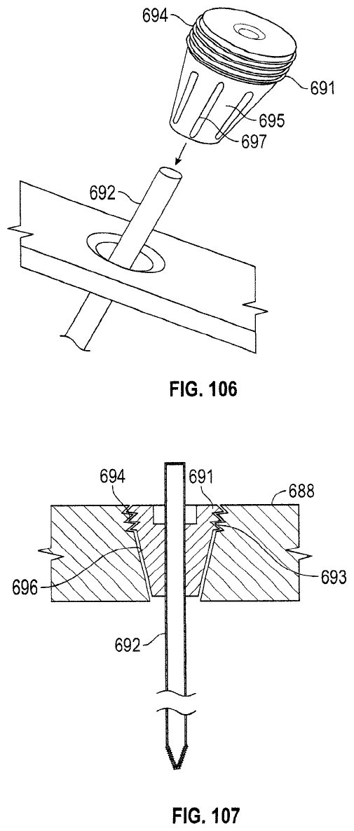

FIGS. 67A-67K illustrate several different manners of moving a bone for cortex distraction, or for moving a plate or other element for periosteal distraction and stretching. One or more of the apparatus and methods disclosed herein can be utilized to achieve any of these movements, without departing from the scope of the disclosure. FIG. 67A illustrates parallel distraction between a first element 1021 and a second element 1022. FIG. 67B illustrates angular distraction between a first element 1023 and a second element 1024. FIG. 67C illustrates parallel plane rotation along an arc about a pivot point 1025 between a first element 1026 and a second element 1027. FIG. 67D illustrates angular distraction about a pivot point 1028 between a first element 1029 and a second element 1030. FIG. 67E illustrates an opening/closing book distraction wherein a first element 1031 is angularly distracted about a first pivot point 1032 and a second element 1033 is angularly distracted about a second pivot point 1034. FIG. 37F illustrates a shuttle method wherein a bone fragment 1035 is translated within a window 1036 cut in a bone 1039, that is moved by a linear translation device 1037. FIG. 67G illustrates rotation of a bone fragment 1038 within a circular window 1040 cut in a bone 1039, that is moved by a rotational device 1041. FIG. 67H illustrates rotation about a longitudinal axis 1042 of a bone fragment 1043 within a window 1044 cut in a bone 1039, that is moved by a device 1045. FIGS. 671-67K illustrate a method of cutting and removing a segment 1046 from a bone 1047, to create a first bone piece 1048 and a second bone piece 1049 (FIG. 67I). Fusing the first bone piece 1048 and the second bone piece 1049 together at a callus 1050 (FIG. 67J). And distracting the first bone piece 1048 and the second bone piece 1049 apart from each other (e.g., at about 1 mm per day) to grow the callus 1050′. Any of these techniques can be utilized with the embodiments of the devices described herein, without departing from the scope of the disclosure.

FIGS. 68A-68F illustrate several manners of fixing a device to a bone 1051. FIG. 68A illustrates a bone screw 1052 configured to be threadingly engaged with one or both cortices of the bone 1051. FIG. 68B illustrates a suture needle 1053 and suture thread 1054 configured to insert through the cortex of the bone 1051 and the be tied securely in place. FIG. 68C illustrates a staple 1055 configured to be lockingly driven into the bone 1051. FIG. 68D illustrates a pin or thread 1056 with one or more wire bead 1057 at the end, and one or more stop 1058, for locking a device between two sides of a cortex. FIG. 68E illustrates first and second L-shaped pins 1059 having a locking portion 1060 extending transversely to a shaft portion 1061, for locking to the cortex of the bone 1051. FIG. 68F illustrates first and second J-shaped pins 1062 having a hook portion 1063 extending transversely to a shaft portion 1064, for locking to the cortex of the bone 1051. Any of these techniques can be utilized with the embodiments of the devices described herein, without departing from the scope of the disclosure.

FIG. 69A illustrates a fully-implantable shape-memory-actuator distraction device 1065. The device 1065 comprises base 1199 having two holes 1169 for placement of bone anchors configured to be screwed into a bone. A stage 1067 includes two holes 1168 for placement of bone screws, or other anchors, but can alternatively configured for stretching a periosteum from below. An actuator base 1066 comprises a slanted groove or slit 1068, similar to the slanted grooves 934a-b of the implantable distraction device 920 of FIGS. 63-64. The stage 1067 includes transversely-extending pins 1069 configured to slide within the slanted groove 1068. The stage 1067 is rigidly coupled to a horizontal actuator 1070 comprising a plurality of nitinol wires 1071 configured to be heated for shortening or lengthening to move the actuator base 1066 longitudinally. As the nitinol wires 1071 are shortened, for example, the actuator base 1066 is moved longitudinally (to the left), causing the stage 1067 to be distracted transversely, in an upward direction. A connection element 1072 couples the translational movement from the nitinol wires 1071 to the actuator base 1066. In an alternative embodiment shown in FIGS. 69B-69C, the connection element 1072 comprises a toothed clutch 1072′ that is configured to lock the amount of displacement in place, or to unlock the displacement. The toothed clutch 1072′ is actuated by a nitinol coil actuator 1073. The nitinol coil actuator 1073 comprises a base portion 1074 that is connected to the actuator base 1066 by an extension 1075. An upper plate 1076 of the coil actuator 1073 is connected to an upper toothed plate 1077 and a lower plate 1078 of the coil actuator 1073 is connected to a lower toothed plate 1079. A central toothed plate 1080 is connects the extension 1075 to the base portion 1074.

In use, the conditional of the stage 1067, in terms of amount of transverse displacement, can be moved from a locked position (FIG. 69B) to an unlocked position (FIG. 69C) by the heating of nitinol coils 1081a, 1081b to cause the coils to increase in length. Alternatively, the coils can be configured to shorten in length from being heated. Thus, they would move from the unlocked position to the locked position. In some embodiments, two-way shape memory is utilized to move back and forth between the locked and unlocked position. A battery powers a regulating circuit that is configured to drive current through one or more conductor electrically connected to the plurality of nitinol wires 1071 and/or the nitinol coils 1081a-b. An external controller can be configured to send signals to the circuit via a transmitter or transceiver. Alternatively, an external coil can heat the nitinol wires 1071 and/or nitinol coils 1081a-b inductively. The external coil can include a controller for controlling the operation of the device 1065, from a location external to the patient. This is explained in more detail in the relation to the device 990 of FIG. 104.

FIG. 70 illustrates a system 1082 similar to the system 100 of FIGS. 17-19. The distraction device 1083 comprises a base 1084 having holes 1085a-b for placing bone anchors for attachment to a bone. A translatable stage 1086 comprises holes 1087a-b for placement of bone anchors for attachment to a bone fragment. The stage 1086 is driven by a leadscrew 1088 that is configured to be turned within the base 1084 without a change to its vertical location, thus driving the stage 1086 in a transverse direction in relation to the bone. The modular automated drive unit 1089 comprises a hex driver 1090 driven by a motor 1091 and a 90° drive train 1092 (bevel gears, worm gears, etc.). A printed circuit board 1093 and a battery 1094 that powers it are carried within the base 1084. The base 1084 comprises a low-profile shape. A center section 1095 has a maximum height to accommodate the necessary height of the leadscrew 1088. However, the side portions 1096a, 1096b, which extend the majority of the length of the base 1084 have minimal profile. The modular automated drive unit 1089 also has a minimized profile and a bottom contour 1097 that is configured to match an upper contour 1098 of the base 1084. In some embodiments, the distraction device 1083 is fully implantable, and a small skin wound above the screw drive 1099 of the leadscrew 1088 can be opened (hypodermic needle or scalpel) and closed (suture, cyanoacrylate, butterfly bandage) continually over the treatment period.

FIGS. 71-79 illustrate several additional configurations of external fixation devices for use with embodiments described herein, in addition to the embodiments presented in FIGS. 23-24. The external fixator 1100 of FIG. 71 comprises one or more rings 1101, and is generically represented by a distal ring 1101a and a proximal ring 1101b. The rings 1101 each extend at least partially around the lower leg 7 of the patient 16. One or more longitudinal connecting rods 1102 are coupled to the rings 1101 and are sized and adjusted accordingly for the patient's needs. A transverse bar 1103 is coupled to the connecting rod 1102b and extends inward toward the lower leg 7. The transverse bar 1103 is sufficiently stiff and has a sufficient diameter to remain rigid without significant bending. An implantable distraction device 1104 is implanted within the leg 7, subcutaneously. A pulley system 1105 comprising a first pulley 1106 and a second pulley 1107 are rotationally held by struts 1108a-b that are connected to and extend inwardly from the connecting rods 1102a-b, respectively. A cable 1109 passes through an incision in the skin of the leg 7 and through a small osteotomy 1110 on an opposite side of the bone 601 from the bone fragment 1111. The cable 1109 extends around the two pulleys 1106, 1107. A first, movable portion 1112 of the cable 1109 passes through an interior of the bone 601. A second, movable portion 1113 of the cable 1109 passes externally to the leg 7. A motor drive unit 1114 is held by the transverse bar 1103, and is configured to grip the second, movable portion 1113 of the cable 1109 to move it in a first transverse direction, thus, via the pulleys 1106, 1107, causing the first, movable portion 1112 of the cable 1109 to be moved in a second transverse direction, opposite the first transverse direction. The cable 1109 is secured to the bone fragment 1111 via first and second opposing beads 1115a-b which keep the cable 1109 from sliding through the bone fragment 1111. The motor drive unit 1114 moves the cable 1109 by either a longitudinal actuator that grips the cable 1109, or by an opposing capstan and pinch roller set. In some embodiments, there may be multiple pulleys in order to reduce the force required to drive the cable 1109.

The external fixator 1116 of FIG. 72 comprises one or more rings 1101, and is generically represented by a distal ring 1101a and a proximal ring 1101b. The rings 1101 each extend at least partially around the lower leg 7 of the patient 16. A takeup spool 1117 is configured to wind a cable 1118 around its central cylindrical portion. The takeup spool 1117 is driven for rotational motion by a motor 1119. Both the frame of the takeup spool 1117 and the motor 1119 are carried on the connecting rod 1102a. Thus, the motor 1119 turns the takeup spool 1117 to place tension on the cable 1118, to pull the bone fragment 1120 in a transverse direction, away from the bone 601. A supply spool 1121 is attached to the connecting rod 1102b via its frame. The supply spool 1121 is passive, but can have an adjustable amount of rotational resistance, or in other embodiments, can incorporate one-directional ratcheting, in order to minimize any excess slack. The cable 1118 is pulled through a small osteotomy 1122 in the bone 601. As the motor 1119 drives the takeup spool 1117 to place tension on the cable 1118 and in turn causes the cable 1118 to wrap around the takeup spool 1117, the bone fragment 1120 is caused to distract away from the bone 601, as it is held securely by first and second opposing beads 1115a-b. Some of the cable 1118 that is on the supply spool 1121 is then unspooled as the supply spool 1121 is caused to turn because of the increased tension and shortening length of the cable 1118. In other embodiments, a second motor is carried on the connecting rod 1102b adjacent the supply spool 1121, and is configured to rotated the supply spool 1121 in an opposite direction (while the takeup spool 1117 is not rotated), thus acting as an opposite direction takeup spool, and causing the bone fragment 1120 to move back toward the bone 601. The opposite direction may be desired in any of the embodiments described herein. For example, the user may determine that the bone fragment 1120 has been distracted too much, and should be moved backward, a least a bit. In periosteal distraction, the user may determine that the periosteum has been distracted too much and that the tension in the periosteum should be reduced, at least a bit. In other cases, the procedure itself can include some distraction, followed by some retraction. This can either be a recipe for optimized bone or other growth in distraction of a bone fragment or other growth stimulator. Or, it can be a manner to obtain good results, but to minimize pain, after a portion of time.

The external fixator 1123 of FIG. 73 comprises one or more rings 1101, and is generically represented by a distal ring 1101a and a proximal ring 1101b. The rings 1101 each extend at least partially around the lower leg 7 of the patient 16. The external fixator 1123 also comprises a pulley 1124 driven by a motor 1125 to pull a cable 1126. However, the pulley 1124 and motor 1125 are carried completely by the distal ring 1101a, without being attached to the connecting rod 1102a. Furthermore, in this embodiment, there is no counter tension on the cable 1126. The bone fragment 1127 can be stabilized by the internal soft tissue alone. In other embodiments, some biocompatible material, such as a silicone sheet, can be implanted, to add some compressible resistance against the bone fragment 1127, to cause its movement to be more precision when pulled by the cable 1126.

The external fixator 1128 of FIG. 74 and the external fixator 1129 of FIG. 75 are each similar to the external fixator 1123 of FIG. 73. The external fixator 1128 of FIG. 74 comprises a motor 1130 that drives a transversely translating plate 1131 via a leadscrew 1132. The external fixator 1129 of FIG. 75 comprises a motor 1133 that drives a transversely translating plate 1134 having a rack 1135 via a pinion 1136.

FIGS. 76A-76B illustrate a clamp 1137 configured to couple to a strut or connecting rod 1102a. The clamp 1137 has an attachment portion 1138 for connecting to the connecting rod 1102a. The clamp 1137 further has an offset clamping portion 1139 having a cavity 1140, through which a number of the devices (motors, pulleys, spools, etc.) can be placed and secured. FIG. 77 illustrates a clamp 1141 having holes 1142a-b through which bolts 1143a-b can be inserted. The complementary connecting rod/strut 1144 comprises a series of holes 1145 through which the bolt 1143 can be inserted, to secure the clamp 1141 to the strut 1144 at the desired linear position along the strut 1144.

The external fixator 1146 of FIG. 78 comprises one or more rings 1101, and is generically represented by a distal ring 1101a and a proximal ring 1101b. A multi-portion strut 1147 comprises a first sub-strut 1148 and a second sub-strut 1149. The first sub-strut 1148 and the second sub-strut 1149 are coupled together at an angulated joint 1150. In some embodiments, the angulated joint 1150 is adjustable, and loosenable, and tightenable to form and maintain a particular angulation. A distraction device 1151 is shown clamped onto the first sub-strut 1148 in FIG. 78. In other embodiments, the distraction device 1151 is instead clamped onto the second sub-strut 1149, to provide a different angulation and orientation of the distraction device 1151. In other embodiments, the distraction device 1151 is clamped onto both the first sub-strut 1148 and the second sub-strut 1149.

The external fixator 1152 of FIG. 79 comprises a strut 1153a having a built-in clamp 1154 and distraction/translation unit 1155. The distraction/translation unit 1155 comprises a leadscrew 1156, but in other embodiments can comprise any of the embodiments described herein that can be configured onto a strut with built-in components.

FIG. 81 illustrates a fully implantable, self-contained transverse distraction device 1170 implanted beneath the skin 976 and secured to a bone 601 of a patient. The device 1170 comprises a base 1171 comprising two holes 1172a-b configured for placement of bone screws 1173a-b, respectively, for securing to cortex of the bone 601. A movable stage 1174 is distractable in relation to the base 1171 and comprises two holes 1175a-b for placement of bone screws 1176a-b, respectively, to couple to a bone fragment 1177. The device 1170 shares many similarities with the implantable distraction device 920 of FIGS. 63-64, however the base 1171 is a more symmetric shape. The device 1170 comprises a motor 1178, circuit board 1179 comprising a controller or control circuit, and a lift bracket 1180 comprising a left and right vertically-extending walls 1181 comprising slanted grooves 1182 into which pins 1183 connected to the movable stage 1174 are configured to be forcibly slid by a leadscrew 1184 driven by the motor 1178.

The steps for implanting the self-contained transverse distraction device 1170 within a patient 16 are described in relation to FIGS. 82-86. In step 1, shown in FIG. 82 a longitudinal incision 1185 is made in the skin 14 of the lower leg 7 of the patient 16, at one side of the tibial crest 1186, distal to the tibial tuberosity 1187, or tubercle. The corticotomy will be made in the tibia 1 laterally adjacent to the longitudinal incision 1185. The periosteum is incised and dissected, and the periosteum and muscle are flipped open. FIG. 83 illustrates an enlarged view of a drill/cutting guide 1188. The drill/cutting guide 1188 comprises a body 1189, and four open channels 1190a-d, each extending from a first side 1191. In step 3, the drill/cutting guide 1188 is located under the muscle and periosteum and fixed to the bone using locking screws or pins, for example 3.0 mm Schanz pins 1192a-d, as shown in FIG. 84. The drill/cutting guide 1188 is used to create a corticotomy using multiple unicortical drill holes or a saw, to create a rectangular intercalary bone segment 1194 (FIG. 85). In some embodiments, the bone segment is on the order of 15 mm×40 mm in size. In step 4, as shown in FIG. 85, the drill/cutting guide 1188 has been slid out laterally along the pins 1192a-d, as enabled by the open channels 1190. The self-contained transverse distraction device 1170 is then placed over the pins 1192a-d. In some cases, thin tubing having an inner diameter that closely but smoothly fits the outer diameter of the pin 1192 and an outer diameter that closely but smoothly fits the inner diameter of the holes 1172, 1176 is placed between each pin 1192 and its hole 1172, 1176 to stabilize the transverse distraction device 1170 upon placement.

Turning to FIG. 86, one middle/unicortical pin 1192b is removed first (vertical up arrow), and a locking screw 1193b (FIG. 97) is secured to the bone fragment/intercalary bone segment 1194. This process is repeated one by one, so that the pin 1192c is removed and replaced by a locking screw 1193c, and then pins 1192a and 1192d are removed and replaced by locking screws 1193a and 1193 d, respectively. In step 5, the skin 14 is sutured. The transverse distraction device 1170 can now be operated wirelessly, as described in some of the teachings related to FIG. 46.

Another embodiment of a distraction device 2000 is shown in FIG. 88. The distraction device 2000 comprises a body 2001 having two through holes 2002a-b for the placement of bone screws 2003a-b to anchor to the tibia 1. Turning to FIG. 89, prior to placement of the distraction device 2000 a drill/cutting guide 2000 is utilized to drill the necessary holes in the tibia 1 and make the necessary cuts of the bone fragment 2051. The distraction device 2000 includes a central screw-based adjuster 2052. The drill/cutting guide 2000 comprises a semi-complete, open rectangular cutting guide space 2004 having ends 2006 and 2007 that abut a connection section 2005 on both sides. Thus, when the drill/cutting guide 2000 is in place, most of the bone segment can be accurately cut from the bone. After removing the drill/cutting guide 2000, the remainder portion, at the uncut portion of bone corresponding to the prior location of the connection section 2005 can be easily cut, by cutting a straight line of bone between the ends of the cut corresponding to the prior location of the end 2006 and end 2007. The drill/cutting guide 2000 further comprises guide holes 2053, 2054, 2055 for drilling of bone to prepare for the bone screws.

As a general rule, distraction devices do not have to be fully or even partially retracted before removing from the patient at the end of the treatment period. The fully distracted or mostly distracted devices can be removed from the patient in their present state. However, in some case, it may be desirable to retract the devices somewhat before removal.

FIGS. 90-92 illustrate further steps to using a simplified manual distraction device comprising a support frame 2008. The support frame 2008 comprises a cap 2009 and a retainer ring 2010. At the step of sliding out the drill/cutting guide 1188 laterally along the pins 1192a-d (FIG. 85), the support frame 2008 is then slid in along the pins 1192a-d. Two pins 1192a, 1192d are tightened to the support frame 2008 and the frame is tightened to the retainer ring 2010. The cap 2009 is attached and tightened to the support frame 2008. The single distraction screw 2012 is coupled to the cut bone segment 1194 by pin 2013. A knob 2011 turns a distraction screw 2012 and the cut bone segment 1194 can be distracted out (away from the bone/tibia 1) and retracted back (toward the bone/tibia 1), by turning the knob 2011 in either direction. As shown in FIG. 90, the support frame 2008 differs from many of the other embodiments in that it is only uni-cortical (attached only to the near bone cortex). The simplified design also allows for a very small profile for the patient to have attached externally. The frame 2008 can be reduced to only about 10 mm in thickness. The pins 1192, 2013 can also be replaced by locking screws.

FIGS. 93-96 illustrate four different configurations for distraction devices to distract the periosteum. FIG. 93 illustrates a distraction device 2020 configured to be coupled to a bone 601 via a base 2021 and bone anchors 2022a-b. A lift plate 2023 is configured to distract an underside of the periosteum 5, and is driven by a distraction screw 2024 that is threadingly coupled to the base 2021 and pinned to the lift plate 2023. The distraction screw 2024 can be adjusted manually by turning a knob 2025 rotationally coupled to the proximal end of the distraction screw 2024, or the distraction screw 2024 can be driven by an in-line motor 2026.

FIG. 94 illustrates a distraction device 2027 configured to be coupled to a bone 601 via a base 2028 and bone anchors 2022a-b. A lift plate 2029 is configured to distract an underside of the periosteum 5, and is driven by a distraction screw 2030 that is threadingly coupled to the base 2028. The distraction screw 2030 can be driven by 90° motor 2044 via a rack and pinion 2031.

FIG. 95 illustrates a distraction device 2032 configured to be coupled to a bone 601 via a base 2033 and bone anchors 2022a-b. A lift plate 2034 is configured to distract an underside of the periosteum 5, and is driven by a distraction screw 2035 that is threadingly coupled to the lift plate 2034 and pinned to the base 2033. The distraction screw 2035 can be adjusted manually by turning a knob 2036 rotationally coupled to the proximal end of the distraction screw 2035, or the distraction screw 2035 can be driven by an in-line motor 2037.

FIG. 96 illustrates a distraction device 2038 configured to be coupled to a bone 601 via a base 2039 and bone anchors 2022a-b. A lift plate 2040 is configured to distract an underside of the periosteum 5, and is driven by a motor 2041 that drives a spool 2042 that puts tension on a tensile wire 2043 to pull the lift plate 2040 against the periosteum 5.

3) Power Source.

A variety of different power sources can be used to operate the device to produce an output torque to drive the mechanism.

### —Manual.

In some embodiments, torque to activate the distraction mechanisms can be produced manually. The starting gear can be rotated by manual force either by connection to a knob or to a wheel. Tools can also be used to drive the starting gear, for example a male hex driver to drive a female socket in the starting gear, or even a socket drive to drive a male drive connection extending from the starting gear. A knob can be replaced by other structures such as a thumb wheel, a hex driver, etc.

### —Magnetic Actuator.

The starting gear can be rotated by being linked or otherwise coupled to a magnet. The magnet is rotated through a secondary magnetic field to induce rotation of the driving magnet and produce torque to drive the starting gear.
External Magnet—the secondary magnet can be a permanent magnet that couples with the drive magnet. The paired magnet poles can be coupled side to side or end to end in order to match the rotation of the drive magnet with the secondary magnet. An example of such end-to-end magnetic coupling is shown in FIG. 98. Alternative magnetic coupling is shown in FIGS. 99 and 100.
Electromagnet—the drive magnet can also be rotated by using electromagnets to produce alternating magnetic fields. The electromagnet array can configured to be fully circumferential around the drive magnet, or a partial arc in order to induce rotation of the drive magnet.

Inside the external electromagnetic actuator, the coils are wrapped around irons which will generate magnetic fields with the energization/excitation of the coils, and this magnetic field will create the magnetic coupling to actuate the internal magnet.

Closed loop control can provide maximum torque generation on the internal magnet. The coils' current will be controlled real-time by a controller using voltage and/or current control, to deliver the necessary amount of torque to distract/retract the iron rod; and in that case, the real time current can be used as indication/estimation of the real time torque or distraction force. One or multiple Hall effect sensors or any other magnetic field sensor can be utilized to detect the real time position of the internal magnet. The detailed control logic and steps to control such an electromagnetic actuator for internal magnet through magnetic coupling are provided in the diagram in FIGS. 101A-101E.

An alternative configuration of such a external electromagnetic actuator uses two connected poles (an arc), instead of two separate iron blocks, as shown in FIGS. 102A-102E.

In both the embodiment of FIGS. 101A-101E and the embodiment of FIGS. 102A-102E, a representative routine for clockwise rotation is shown in Table, below. After Step 4, Step 1 repeats, and the cycle continues.

TABLE 1

| | Initiation | | Idle | | | Idle | |
|---|---|---|---|---|---|---|---|
| | (T = 0) | Step 1 | (<1 second) | Step 2 | Step 3 | (<1 second) | Step 4 |
| Coil 1 | S | N | Hall effect | N | S | Hall effect | S |
| | | | sensor | | | sensor | |
| | | | reading | | | reading | |
| Coil 2 | S | S | Hall effect | N | N | Hall effect | S |
| | | | sensor | | | sensor | |
| | | | reading | | | reading | |

### —Motor Driven.

The starting gear can be rotated by an electric motor (DC, stepper, or AC, etc.)
Inductive Power Transfer—the electric motor can be powered wirelessly through inductive power transmission.
Battery—the electric motor can be powered by being connected to a battery source.

### —Wireless Power Transmission

In these embodiments, two main sets of coils (one internal, the other one external) can be used to transfer power wireless through inductive coupling. The transmitting frequency between the two coils can be tuned via capacitor, or secondary coil to achieve the resonant frequency of the system for the highest transmission efficiency purpose. An internal rechargeable battery or capacitor can be used to store the energy from the wireless power transmission.
Two magnets can be used for transferring rotational energy, as shown in FIG. 103. Inside, coils 985 are wrapped around the internal magnet 986, and through the rotational of an external magnet 987 internal magnet, electric voltage/energy will be generated on the coils 985 based on Faraday's law. The internal magnet 986/coils 985 generally act as a generator. A voltage regulating circuit 988 controls the energy to charge an implanted rechargeable battery 989.

### —Nitinol (Shape-Memory Alloy) Driven.

In addition to actuators that have rotating outputs, nitinol elements can be used to produce linear movements. Nitinol elements can be shortened/lengthened by running current through them to produce resistive heating. This linear movement can be used to drive a ratchet mechanism along a rack in order to push/pull mechanical elements (listed above) to achieve distraction, such as a linkage bar in a closed-bar linkage to create movement. The heating of the Nitinol wires can be achieved by the internal/implant battery. Another power source can be implemented through wireless power transmission between internal and external coils from outside power sources. A fully-implantable shape-memory-actuator distraction device 990 in FIG. 104 comprises a base 991 having two holes (not shown) for placement of bone anchors configured to be screwed into a bone. A stage 992 includes two holes 993a-b for placement of bone screws, but can alternatively configured for stretching a periosteum from below. The stage 992 is statically coupled to a first plate 993. A plurality of nitinol wires 994 are connected between the first plate 993 and the base 991 in a substantially vertical direction. A battery 995 powers a regulating circuit 996 that is configured to drive current through one or more conductor 997 electrically connected to the plurality of nitinol wires 994. An external controller can be configured to send signals to the circuit 996 via a transmitter or transceiver. Alternatively, an external coil 998 can heat the nitinol wires inductively. The external coil 998 can include a controller 899 for controlling the operation of the device 990, from a location external to the patient (outside the skin 976). The controller 899 is coupled to a user interface 999 for ease of operation of the coil 998. The heating an resultant lengthening of the plurality of nitinol wires 994 causes the stage 992 to distract upward. If two-way shape memory is utilized, the cooling of the plurality of nitinol wires can also retract the stage 992.

### —Spring or Other Passive Energy Storage/Release Methods Such as Coils or Leaves, Etc.

In addition to the active energy storage methods described above, passive energy storage methods such as springs, coils, or leaves can be used to push the bone fragment in one direction. When used in combination with a reciprocating mechanism such as a slider crank or scotch yoke, after reaching the extent of distraction in one direction, the continuation of the spring mechanism can push the bone fragment in the opposite direction/retraction.
Energy Harvesting can also be used by making use of human motion to load up the spring/coils to store energy, or charge batteries, etc.
Piezo actuators, or pneumatic, or hydraulic actuators where the power sources come from piezoelectric elements, or pressurized air/gasses, or pressurized liquids such as oil or water, respectively.

(4) Additional Technology.

### —Device Communication—

Wireless—the device can be connected to an external controller through wireless means, including, but not limited to, the following: WiFi, Bluetooth or BLE, NFC, and RFID. One example of wireless communication is an implementation in which an external controller is wirelessly connected to the device in order to initiate actuation, as well as to send and receive data from the device. The external controller can be a smart phone where users can interact with a customized controller App for communication and actuation with the device where the driver/actuator will be on site with the device. In another embodiment, an internal controller can be implanted proximate to the implanted TTT device or PD device (all appropriately sealed). The internal controller can not only regulate the operation of the TTT device or PD device itself but also send and receive diagnostic and operational data from the implant to an external or remote device through wireless communication methods as described above.
Wired—the device can be directly connected to a controller if it is external for sending and receiving data via wired connections, e.g., a micro-USB port, or through some transferable memory storage devices such as a USB flash drive, or an SD card.

### —Prescriptions for Automated Distraction.

The medical professional (physician, surgeon, nurse practitioner, physician's assistant, etc.) can set the prescription for the distraction protocol. Actuations can be performed by using the controller to initiate actuations, executed by the patient at various times throughout the day. The controller can also be programmed based on the prescription to automatically send commands throughout the day to initiate distractions without requiring any action from the patient.
In some embodiments, the prescription can also be loaded wirelessly onto the device through the methods as described above so that the device will execute the distraction program autonomously without needing to be connected to the controller. This allows for the actuator to initiate distractions more frequently throughout the day without inconveniencing the patient, or even for continuous distraction throughout the prescription duration.

### —Integrated Biosensors.

The device can include sensors or estimators to record information about its environment or activity of the patient. The types of sensors that can be used include, but are not limited to, temperature, distraction distance, forces, mechanical force (tension/compression, torsion, pressure), pH, moisture, biomarkers (glucose, proteins, bacteria, acid, etc.), and accelerometers to measure movements and position.
   —Cloud based data.
Data that is retrieved from the device onto the controller can be uploaded into the cloud. This allows remote monitoring of the recorded data from the device, such as the progress of the treatment regimen or sensor data. The two-way data communication can be between the controller/device and the cloud, or the user controller App/phone and the cloud, or a combination thereof.
The cloud-based connectivity of the controller also allows for a medical professional to make adjustments to the device remotely. For example, if the sensor data indicates that the distraction forces are increasing, the distraction rate can be increased in order to prevent premature consolidation (of the bone or bones). An exemplary network-based monitoring and control configuration for use with the embodiments presented herein and methods is provided in the diagram shown in FIG. 46.

### —Bone Fixation Screws/Pins

Certain external fixation systems rely on a clamp system on the fixator frame to secure around the diameter of the shaft of the fixation screw-pin in order to provide rigid fixation of the bone. The device includes the use of external threads in the screw head to mate with the threads in the external frame to provide the locking and rigid connection between the fixation screw and the frame. This is beneficial because the fixation elements remain flush with the frame so that the construct does not have long protruding fixator pins away from the patient's body. FIG. 105 illustrates an example of this approach. A screw 683 comprises a shaft 684, and distal threaded portion 685, and a head 686 having an external thread 687. A plate 688 (or frame) comprises an internally-threaded hole 689 that is configured to allow the distal threaded portion 685 and the shaft 684 to pass therethrough, and is also configured to threadingly engage the external thread 687 of the head 686. In some embodiments, the external thread 687 of the head 686 is configured to lock to the internally-threaded hole 689. As shown in FIG. 105, the head 686 is flush with the plate 688 surface 690 when locked in place, thus providing a low-profile assembly.

The device can also use fixation pins for fixation of the bone. The rigid connection between the pin (or screw) and the frame can include a collet cap device 691 (FIGS. 106-107) to frictionally lock around the diameter of the pin shaft 692, as shown in FIG. 107. The external taper 695 of the collet cap device 691 is wedged into a tapered hole 696 of the frame 688. This is beneficial because the plate/frame 688 can utilize the internal locking thread 693 for locking the external thread 694 fixation screws, mentioned in the previous paragraph, or can utilize more standard fixator pins. There are a series of slots 697 that extend longitudinally in the collet cap 691 and facilitate the wedging during the tightening of the external thread 694 to the internal locking thread 693. The bottom of the collet cap can include a slot cut pattern (e.g., star pattern) to add frictional fit with the pin 692.

Any of the embodiments described herein can supply the motion to move either a bone fragment, or a growth-inducing material, or can drive a plate or other member to distract and stretch a periosteum. Each embodiment can be modified to provide a completely implantable device, or a device that is partially planted (distraction portion) and partially external (driving portion).

While the foregoing is directed to embodiments of the present disclosure, other and further embodiments may be devised without departing from the basic scope thereof.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

For purposes of the present disclosure and appended claims, the conjunction “or” is to be construed inclusively (e.g., “an apple or an orange” would be interpreted as “an apple, or an orange, or both”; e.g., “an apple, an orange, or an avocado” would be interpreted as “an apple, or an orange, or an avocado, or any two, or all three”), unless: (i) it is explicitly stated otherwise, e.g., by use of “either . . . or,” “only one of,” or similar language; or (ii) two or more of the listed alternatives are mutually exclusive within the particular context, in which case “or” would encompass only those combinations involving non-mutually-exclusive alternatives. For purposes of the present disclosure and appended claims, the words “comprising,” “including,” “having,” and variants thereof, wherever they appear, shall be construed as open-ended terminology, with the same meaning as if the phrase “at least” were appended after each instance thereof.

1-3. (canceled)

4. A transport device for increasing biological activity within a patient, comprising:
a base comprising a first end, a second end, an upper surface located between the first end and the second end, and a lower surface located between the first end and the second end;
a base anchor configured to statically couple the base to a first portion of a bone of a subject;
a translatable anchor configured to engage a growth stimulator;
a leadscrew dynamically coupling the base to the translatable anchor, wherein the leadscrew is configured to rotate about a leadscrew axis while substantially maintaining its longitudinal position along the leadscrew axis in relation to the base, such that the growth stimulator, when engaged with the translatable anchor, is capable of independent movement in relation to the base along a stimulation axis that includes at least some transverse displacement with respect to the bone when the base is coupled to the bone via the base anchor;
a screw drive rotatably coupled to the leadscrew and configured to couple to a rotatable mating tool that is configured to rotate the leadscrew via the screw drive;
a drive unit comprising and configured to cause rotation of the rotatable mating tool, the drive unit configured to be couplable to and uncouplable from the base such that, when coupled, the rotatable mating tool is coupled to the screw drive and configured to rotate the leadscrew to move the translatable anchor in relation to the base, wherein the base comprises an indexing contour configured to substantially stabilize the base in relation to the drive unit in at least one degree of freedom; and
a locking portion configured to enable the base to be locked to the drive unit.

5. The transport device of claim 4, wherein the indexing contour comprises at least one of a pin or a hole, and wherein the locking portion comprises an internally-threaded hole configured to be engaged by a locking screw.

6-76. (canceled)
